(19) United States
(12) Patent Application Publication
(10) Pub. No.: US 2025/0114382 A1
(43) Pub. Date: Apr. 10, 2025

(54) ENHANCED CAPTURE AND DISSOLUTION MATRIX FOR CANNABINOIDS AND METHODS OF MAKING THE SAME

(71) Applicant: Ilera Therapeutics LLC, Newtown Square, PA (US)

(72) Inventors: Oludare ODUMOSU, Newtown Square, PA (US); Christopher HILL, Newtown Square, PA (US)

(21) Appl. No.: 18/988,155

(22) Filed: Dec. 19, 2024

Related U.S. Application Data
(63) Continuation of application No. PCT/US2023/068501, filed on Jun. 15, 2023.
(60) Provisional application No. 63/354,453, filed on Jun. 22, 2022.

(51) Int. Cl. A61K 31/00; A61K 9/16; A61K 9/20; A61K 9/48; A61K 36/185

(52) U.S. Cl. CPC A61K 31/658; A61K 9/1682; A61K 9/2009; A61K 9/2018; A61K 9/2054; A61K 9/2059; A61K 9/485; A61K 9/4866; A61K 36/185

(57) ABSTRACT

The present disclosure provides compositions containing Cannabinoid Products and methods of making the same. The disclosure also provides tablets and capsules containing Cannabinoid Products and methods of making the tablets and capsules.

### CROSS-REFERENCE TO RELATED APPLICATION

This application is a continuation of International Application No. PCT/US2023/068501, filed Jun. 15, 2023, which claims priority to U.S. Provisional No. 63/354,453, filed Jun. 22, 2022. This application is incorporated by reference herein in its entirety.

### FIELD

The present disclosure provides solid Cannabinoid Products, including tablets and capsules. The disclosure also provides methods of making said Cannabinoid Products, tablets and capsules.

### BACKGROUND

Cannabis has over 80 active constituents with potential therapeutic benefits. For instance, research shows that cannabinoids isolated from Cannabis stabilize blood sugar, provide relief from chronic pain, help with weight loss, improve depression and anxiety, prevent seizures, and allow broken bones to heal more quickly.

Oral pharmaceutical compositions, for example, tablets and capsules, which comprise cannabinoids, are desirable for the treatment of diseases. Tablets are prepared by compressing a powder containing cannabinoids using a tablet press. Capsules are prepared by placing the powder containing cannabinoids in a shell, for example, a gelatin shell. Many solid cannabinoid products are formulated using crystalline (e.g., precipitated, powder) forms of cannabinoid. These products, however, can suffer from low bioavailability when ingested. Unfortunately, formulating tablets and capsules containing cannabinoids is challenging because certain forms of non-crystalline cannabinoids, such non-crystalline cannabinoid oils, are viscous and oily, and not readily integrated into solid formulations. The high viscosity of cannabinoids results in separation of the cannabinoids from the powder. Additionally, cannabinoids are insoluble in water and thus difficult to formulate. Thus, there is a need in the art for the formulation of oral pharmaceutical compositions containing cannabinoids, which are stable, bioavailable, and uniformly dispersed in the powder.

### SUMMARY

The present disclosure provides Cannabinoid Products containing cannabinoids and methods of making Cannabinoid Products. The disclosure also provides tablets and capsules comprising Cannabinoid Products and methods of making the tablets and capsules.

Provided herein is a Cannabinoid Product comprising: (a) a non-crystalline cannabinoid oil; and (b) colloidal silicon dioxide particles. Provided herein is a Cannabinoid Product comprising: (a) a non-crystalline cannabinoid oil; (b) colloidal silicon dioxide particles; (c) a filler comprising silicified microcrystalline cellulose or mannitol; (d) a disintegrant comprising sodium starch glycolate or croscarmellose sodium; and (e) a lubricant comprising sodium stearyl fumarate. Provided herein is a Cannabinoid Product comprising: (a) a non-crystalline cannabinoid oil; (b) colloidal silicon dioxide particles; and (c) a lubricant comprising sodium stearyl fumarate. In embodiments, the Cannabinoid Product comprises from about 10% to about 60% non-crystalline cannabinoid oil by weight. In embodiments, the Cannabinoid Product comprises from about 0.5% to about 20% non-crystalline cannabinoid oil by weight. In embodiments, the Cannabinoid Product comprises from about 1% to about 10% non-crystalline cannabinoid oil by weight. In embodiments, the Cannabinoid Product comprises about 2% non-crystalline cannabinoid oil by weight. In embodiments, the Cannabinoid Product comprises about 8% non-crystalline cannabinoid oil by weight. In embodiments, the Cannabinoid Product comprises from about 19.4% to about 41.9% non-crystalline cannabinoid oil by weight. In embodiments, the filler is silicified microcrystalline cellulose. In embodiments, the Cannabinoid Product comprises from about 30% to about 60% silicified microcrystalline cellulose by weight. In embodiments, the Cannabinoid Product comprises from about 20% to about 45% silicified microcrystalline cellulose by weight. In embodiments, the Cannabinoid Product comprises about 46.6% silicified microcrystalline cellulose by weight. In embodiments, the Cannabinoid Product comprises about 25% silicified microcrystalline cellulose by weight. In embodiments, the filler is mannitol. In embodiments, the Cannabinoid Product comprises from about 5% to about 60% mannitol by weight. In embodiments, the Cannabinoid Product comprises from about 12.3% to about 43.4% mannitol by weight. In embodiments, the Cannabinoid Product comprises from about 20% to about 70% colloidal silicon dioxide particles by weight. In embodiments, the Cannabinoid Product comprises from about 29.1% to about 58.2% colloidal silicon dioxide particles by weight. In embodiments, the Cannabinoid Product comprises from about 25% to about 50%, from about 25% to about 35%, or from about 35% to about 45% colloidal silicon dioxide particles by weight. In embodiments, the Cannabinoid Product comprises about 32% colloidal silicon dioxide particles by weight. In embodiments, the Cannabinoid Product comprises about 39% colloidal silicon dioxide particles by weight. In embodiments, the Cannabinoid Product comprises a disintegrant. In embodiments, the disintegrant is sodium starch glycolate. In embodiments, the Cannabinoid Product comprises from about 0.5% to about 10% sodium starch glycolate by weight. In embodiments, the Cannabinoid Product comprises from about 2.0% to about 5.5% sodium starch glycolate by weight. In embodiments, the disintegrant is croscarmellose sodium. In embodiments, the Cannabinoid Product comprises from about 0.5% to about 5% croscarmellose sodium by weight. In embodiments, the Cannabinoid Product comprises about 1.9% croscarmellose sodium by weight. In embodiments, the Cannabinoid Product comprises from about 0.5% to about 10% sodium stearyl fumarate by weight. In embodiments, the Cannabinoid Product comprises from about 1% to about 2.7% sodium stearyl fumarate by weight. In embodiments, the Cannabinoid Product comprises from about 0.1% to about 1% sodium stearyl fumarate by weight. In embodiments, the Cannabinoid Product comprises about 0.4% sodium stearyl fumarate by weight. In embodiments, the Cannabinoid Product comprises. In embodiments, the Cannabinoid Product comprises: (a) about 41.9% non-crystalline cannabinoid oil by weight; (b) about 41.9% colloidal silicon dioxide particles by weight; (c) about 12.3% mannitol by weight; (d) about 2% sodium starch glycolate by weight; and (e) about 2% sodium stearyl fumarate by weight. In embodiments, the Cannabinoid Product comprises: (a) about 22.1% non-crystalline cannabinoid oil by weight; (b) about 58.2% colloidal silicon dioxide particles by weight; (c) about 14.3% mannitol by weight; (d) about 2.7% sodium starch glycolate by weight; and (e) about 2.7% sodium stearyl fumarate by weight. In embodiments, the Cannabinoid Product comprises: (a) about 22.1% non-crystalline cannabinoid oil by weight; (b) about 29.1% colloidal silicon dioxide particles by weight; (c) about 40.7% mannitol by weight; (d) about 5.5% sodium starch glycolate by weight; and (e) about 2.7% sodium stearyl fumarate by weight. In embodiments, the Cannabinoid Product comprises. In embodiments, the Cannabinoid Product comprises (a) about 22.1% non-crystalline cannabinoid oil by weight; (b) about 29.1% colloidal silicon dioxide particles by weight; (c) about 43.4% mannitol by weight; (d) about 2.7% sodium starch glycolate by weight; and (e) about 2.7% sodium stearyl fumarate by weight. In embodiments, the Cannabinoid Product comprises: (a) about 19.4% non-crystalline cannabinoid oil by weight; (b) about 31.1% colloidal silicon dioxide particles by weight; (c) about 46.6% silicified microcrystalline cellulose by weight; (d) about 1.9% croscarmellose sodium by weight; and (e) about 1% sodium stearyl fumarate by weight. In embodiments, the Cannabinoid Product comprises: (a) about 2.2% non-crystalline cannabinoid oil by weight; (b) about 32.4% colloidal silicon dioxide particles by weight; (c) about 0.4% sodium stearyl fumarate by weight; and (d) about 34.8% silicified microcrystalline cellulose by weight. In embodiments, the Cannabinoid Product comprises: (a) about 8.1% non-crystalline cannabinoid oil by weight; (b) about 39% colloidal silicon dioxide particles by weight; (c) about 0.4% sodium stearyl fumarate by weight; (d) about 40.4% cannabidiol (CBD) isolate by weight; and (e) about 11.7% cannabigerol (CBG) isolate by weight. In embodiments, the Cannabinoid Product comprises: (a) about 8.1% non-crystalline cannabinoid oil by weight; (b) about 39% colloidal silicon dioxide particles by weight; (c) about 0.4% sodium stearyl fumarate by weight; (d) about 40.4% cannabidiol (CBD) isolate by weight; (e) about 11.7% cannabigerol (CBG) isolate by weight; (f) about 0.2% myrcene by weight; and (g) about 0.2% linalool by weight. In embodiments, the Cannabinoid Product comprises: (a) about 2.2% non-crystalline cannabinoid oil by weight of the Cannabinoid Product; (b) about 32.4% colloidal silicon dioxide particles by weight; and (c) about 0.4% sodium stearyl fumarate by weight. In embodiments, the Cannabinoid Product comprises: (a) about 8.1% non-crystalline cannabinoid oil by weight; (b) about 39% colloidal silicon dioxide particles by weight; and (c) about 0.4% sodium stearyl fumarate by weight. In embodiments, the Cannabinoid Product comprises a cannabinoid isolate. In embodiments, the cannabinoid isolate is cannabidiol (CBD) isolate or cannabigerol (CBG) isolate. In embodiments, the Cannabinoid Product comprises from about 20% to about 60%, from about 20% to about 40%, or from about 40% to about 60% cannabinoid isolate by weight. In embodiments, the Cannabinoid Product comprises a flavoring. In embodiments, the flavoring is a terpene. In embodiments, the terpene is myrcene, linalool, or a combination thereof. In embodiments, the non-crystalline cannabinoid oil is a non-crystalline cannabinoid oil. In embodiments, provided herein is a Cannabinoid Product comprising a non-crystalline cannabinoid oil, colloidal silicon dioxide particles, and optionally filler, disintegrant, or lubricant, wherein the non-crystalline cannabinoid oil and the colloidal silicon dioxide particles are mixed in a high-shear mixer before the addition of the filler, disintegrant, or lubricant. In embodiments, provided herein is a Cannabinoid Product comprising a non-crystalline cannabinoid oil, colloidal silicon dioxide particles, and a lubricant; wherein the non-crystalline cannabinoid oil and the colloidal silicon dioxide particles are mixed in a high-shear mixer before the addition of the lubricant. In embodiments, provided herein is a Cannabinoid Product; wherein the lubricant is added after the filler and disintegrant. In embodiments, provided herein is a Cannabinoid Product; wherein the lubricant is added after the non-crystalline cannabinoid oil and colloidal silicon dioxide particles. In embodiments, the colloidal silicon dioxide particles of a Cannabinoid Product described herein have an average particle size of 20-60 μm. In embodiments, the colloidal silicon dioxide particles of a Cannabinoid Product described herein contain pores with a volume of 1.5-1.9 milliliter (mL)/gram (g).

In embodiments, provided herein is a tablet comprising any Cannabinoid Product described herein. In embodiments, the tablet has a hardness of from 50 Newtons (N) to 100 N. In embodiments, the tablet has a hardness of about 65 N. In embodiments, the tablet has a compression force of from 1 kiloNewtons (kN) to 30 kN. In embodiments, the tablet has a compression force of from about 10 kN to 16 kN. In embodiments, at least 18% of the tablet dissolves in fed state simulated intestinal fluid or fasted state simulated intestinal fluid within one hour. In embodiments, from 60% to about 80% of the tablet dissolves in fed state simulated intestinal fluid or fasted state simulated intestinal fluid within one hour. In embodiments, from 70% to about 80% of the tablet dissolves in fed state simulated intestinal fluid or fasted state simulated intestinal fluid within one hour. In embodiments, at least 36.5% of the tablet dissolves in polysorbate 80 in one hour. In embodiments, the tablet disintegrates in from 1 minute to about 10 minutes in 2% w/v sodium lauryl sulfate. In embodiments, the tablet dissolves at least two times, at least three times, at least four times, at least five times, or at least ten times faster than a tablet containing a Cannabinoid Product lacking colloidal silicon dioxide particles. In embodiments, the tablet has a weight of from about 100 to about 800 mg. In embodiments, the tablet comprises from about 0.1% to about 30% of non-crystalline cannabinoid oil by weight. In embodiments, the tablet is an orodispersable tablet.

In embodiments, provided herein is a capsule comprising any Cannabinoid Product described herein. In embodiments, the capsule comprises a shell comprising gelatin or hydroxypropylmethylcellulose. In embodiments, the capsule disintegrates within about 2 minutes in an aqueous solvent. In embodiments, at least 18% of the capsule dissolves in fed state simulated intestinal fluid or fasted state simulated intestinal fluid within one hour. In embodiments, from 18% to about 60% of the capsule dissolves within one hour in fed state simulated intestinal fluid or fasted state simulated intestinal fluid. In embodiments, from 18% to about 42.3% of the capsule dissolves within one hour in fed state simulated intestinal fluid or fasted state simulated intestinal fluid. In embodiments, at least 36.5% of the capsule dissolves in fed state simulated intestinal fluid or fasted state simulated intestinal fluid within one hour. In embodiments, the capsule dissolves at least two times, at least three times, at least four times, at least five times, or at least ten times faster than a capsule containing a Cannabinoid Product lacking colloidal silicon dioxide particles. In embodiments, the capsule comprises from about 1% to about 50% non-crystalline cannabinoid oil by weight.

In embodiments, provided herein is a method of making a Cannabinoid Product described herein, comprising: (a) mixing the non-crystalline cannabinoid oil and colloidal silicon dioxide particles; (b) adding the filler and disintegrant to the mixture of non-crystalline cannabinoid oil and colloidal silicon dioxide particles; and (c) adding the lubricant. In embodiments, step (a) is performed before step (b), and step (b) is performed before step (c). In embodiments, the non-crystalline cannabinoid oil is heated before the extract oil is mixed with the colloidal silicon dioxide particles. In embodiments, the non-crystalline cannabinoid oil and colloidal silicon dioxide particles are heated after mixing. In embodiments, the non-crystalline cannabinoid oil is diluted in a solvent before mixing with colloidal silicon dioxide particles. In embodiments, the solvent is ethanol. In embodiments, the non-crystalline cannabinoid oil is heated to at least 30° C., at least 40° C., at least 50° C., at least 60° C., at least 70° C., at least 80° C., or at least 90° C. In embodiments, the non-crystalline cannabinoid oil is added to the colloidal silicon dioxide particles. In embodiments, the colloidal silicon dioxide particles are added to the non-crystalline cannabinoid oil. In embodiments, the non-crystalline cannabinoid oil and colloidal silicon dioxide particles are mixed in a high-shear mixer. In embodiments, the non-crystalline cannabinoid oil and colloidal silicon dioxide particles are mixed for at least 30 seconds, at least 1 minute, at least 5 minutes, at least 10 minutes, at least 15 minutes, or at least 20 minutes. In embodiments, the non-crystalline cannabinoid oil and colloidal silicon dioxide particles are mixed at from 1000 to 2000 revolutions per minute (rpm). In embodiments, the high-shear mixer is a grinder. In embodiments, provided herein is a method of making a tablet comprising compressing a Cannabinoid Product described herein in a tablet press. In embodiments, the tablet is about 100 to 800 mg. In embodiments, the tablet comprises from about 0.1% to about 30% of non-crystalline cannabinoid oil by weight. In embodiments, the tablet is an orodispersable tablet. In embodiments, provided herein is a method of making a Cannabinoid Product described herein comprising: (a) mixing the non-crystalline cannabinoid oil and colloidal silicon dioxide particles; and (b) adding the lubricant. In embodiments, the method further comprises (c) adding a filler. In embodiments, the filler is silicified microcrystalline cellulose. In embodiments, the non-crystalline cannabinoid oil is diluted in a solvent before mixing with colloidal silicon dioxide particles. In embodiments, the solvent is ethanol. In embodiments, step (a) is performed before step (b). In embodiments, step (a) is performed before step (b), and step (b) is performed before step (c). In embodiments, step (a) is performed before step (c), and step (c) is performed before step (b). In embodiments, the method comprises heating the non-crystalline cannabinoid oil before the oil is mixed with the colloidal silicon dioxide particles. In embodiments, the non-crystalline cannabinoid oil and colloidal silicon dioxide particles are heated after mixing. In embodiments, the method comprises heating the non-crystalline cannabinoid oil to at least 30° C., at least 40° C., at least 50° C., at least 60° C., at least 70° C., at least 80° C., or at least 90° C. In embodiments, the method comprises adding the non-crystalline cannabinoid oil to the colloidal silicon dioxide particles. In embodiments, the method comprises adding the colloidal silicon dioxide particles to the non-crystalline cannabinoid oil. In embodiments, the method comprises mixing the non-crystalline cannabinoid oil and colloidal silicon dioxide particles in a high-shear mixer. In embodiments, the method comprises mixing the non-crystalline cannabinoid oil and colloidal silicon dioxide particles in a high-shear mixer for at least 30 seconds, at least 1 minute, at least 5 minutes, at least 10 minutes, at least 15 minutes, or at least 20 minutes. In embodiments, the method comprises mixing the non-crystalline cannabinoid oil and colloidal silicon dioxide particles at a speed of from about 1000 revolutions per minute (rpm) to about 2000 rpm. In embodiments, the method comprises mixing the lubricant with a mixture containing non-crystalline cannabinoid oil, colloidal silicon dioxide particles, filler, and disintegrant for from 1 minute (min) to about 2 min. In embodiments, the high-shear mixer is a grinder. In embodiments, provided herein is a capsule comprising filling a capsule shell with a Cannabinoid Product described herein. In embodiments, the capsule shell comprises gelatin or hydroxypropylmethylcellulose. In embodiments, the capsule comprises from about 1% to about 50% non-crystalline cannabinoid oil by weight.

In embodiments, provided herein is a Cannabinoid Product described herein comprising non-crystalline cannabinoid oil and colloidal silicon dioxide particles comprising internal storage cavities. In embodiments, at least 90% of the non-crystalline cannabinoid oil is loaded into the colloidal silicon dioxide storage particles, and wherein the loaded non-crystalline cannabinoid oil to colloidal silicon dioxide particle ratio is about 0.5:1 to 3:1 by weight. In embodiments, the non-crystalline cannabinoid oil in the Cannabinoid Product is in solid form. In embodiments, the non-crystalline cannabinoid oil is not diluted in a non-cannabis solvent. In embodiments, the non-cannabis solvent is an alcohol. In embodiments, the non-cannabis solvent is medium-chain triglycerides. In embodiments, the non-cannabis solvent is castor oil. In embodiments, the non-cannabis solvent is Kolliphor EL. In embodiments, the loaded non-crystalline cannabinoid oil to colloidal silicon dioxide particle ratio is about 1:1 to 2:1 by weight. In embodiments, the loaded non-crystalline cannabinoid oil to colloidal silicon dioxide particle ratio is about 1:5 by weight.

In embodiments, provided herein is a Cannabinoid Product described herein comprising non-crystalline cannabinoid oil and colloidal silicon dioxide particles comprising internal storage cavities. In embodiments, at least 90% of the non-crystalline cannabinoid oil is loaded into the colloidal silicon dioxide storage particles, and wherein the non-crystalline cannabinoid oil comprises at least 30% w/w of the colloidal silicon dioxide particles. In embodiments, the non-crystalline cannabinoid oil is not diluted in a non-cannabis solvent. In embodiments, wherein the non-cannabis solvent is an alcohol. In embodiments, the non-cannabis solvent is medium-chain triglycerides. In embodiments, the non-cannabis solvent is castor oil. In embodiments, the non-cannabis solvent is Kolliphor EL. In embodiments, the non-crystalline cannabinoid oil comprises at least 50% w/w of the colloidal silicon dioxide particles comprising internal storage cavities. In embodiments, the non-crystalline cannabinoid oil comprises at least 70% w/w of the colloidal silicon dioxide particles comprising internal storage cavities.

In embodiments, provided herein is a cannabinoid daily dose comprising: (a) non-crystalline cannabinoid oil comprising a cannabinoid; and (b) colloidal silicon dioxide particles comprising internal storage cavities. In embodiments, at least 90% of the non-crystalline cannabinoid oil is loaded into the colloidal silicon dioxide particles comprising internal storage cavities, and wherein said daily dose comprises greater than 50 mg of the cannabinoid. In embodiments, the daily dose is a pill. In embodiments, the daily dose is a tablet. In embodiments, the daily dose is a single pill. In embodiments, the single pill comprises about 75 mg to about 150 mg cannabinoid. In embodiments, the daily dose is at least two pills. In embodiments, the daily dose is a single tablet. In embodiments, the single tablet comprises about 75 mg to about 150 mg cannabinoid. In embodiments, the daily dose is at least two tablets. In embodiments, the daily dose comprises about 0.1 mg/kg/day to about 25 mg/kg/day of cannabinoid. In embodiments, the daily dose comprises about 1 mg/kg/day of non-crystalline cannabinoid oil. In embodiments, the single pill comprises about 50 mg to about 100 mg colloidal silicon dioxide particles comprising internal storage cavities. In embodiments, the single tablet comprises about 50 mg to about 100 mg colloidal silicon dioxide particles comprising internal storage cavities. In embodiments, the non-crystalline cannabinoid oil in the Cannabinoid Product is in solid form. In embodiments, the non-crystalline cannabinoid oil is not diluted in a non-cannabis solvent. In embodiments, the non-cannabis solvent is an alcohol. In embodiments, the non-cannabis solvent is medium-chain triglycerides. In embodiments, the non-cannabis solvent is castor oil. In embodiments, the non-cannabis solvent is Kolliphor EL.

In embodiments, provided herein is a Cannabinoid Product described herein comprising: (a) non-crystalline cannabinoid oil; and (b) colloidal silicon dioxide particles. In embodiments, at least 90% of the non-crystalline cannabinoid oil is loaded into the colloidal silicon dioxide storage particles. In embodiments, the product exhibits a linear dissolution rate as measured in 1% Tween 80 media, Fasted State Simulated Intestinal Fluid (FaSSIF) media, and/or Fed State Simulated Intestinal Fluid (FeSSIF) media. In embodiments, the dissolution rate remains linear throughout 5-60 minutes after contacting the product with the 1% tween 80 media, FaSSIF media, and/or FeSSIF media. In embodiments, the dissolution rate remains linear throughout a first phase, a second phase, and third phase after contacting the product with the 1% tween 80 media, FaSSIF media, and/or FeSSIF media. In embodiments, the first phase comprises a period of 5-14 minutes after contacting the product with the 1% tween 80 media, FaSSIF media, and/or FeSSIF media, wherein the second phase comprises a period of 15-34 minutes after contacting the product with the 1% tween 80 media, FaSSIF media, and/or FeSSIF media, and wherein the third phase comprises a period of 35-60 minutes after contacting the product with the 1% tween 80 media, FaSSIF media, and/or FeSSIF media. In embodiments, the linear dissolution rate comprises a first phase: third phase slope ratio not great than about 5. In embodiments, the Cannabinoid Product exhibits a linear absorption rate for at least 20 minutes after administration. In embodiments, the Cannabinoid Product exhibits a linear absorption rate for at least 40 minutes after administration. In embodiments, the Cannabinoid Product exhibits a linear absorption rate for at least 1 hour after administration. In embodiments, the non-crystalline cannabinoid oil in the Cannabinoid Product is in solid form. In embodiments, the non-crystalline cannabinoid oil is not diluted in a non-cannabis solvent. In embodiments, the non-cannabis solvent is an alcohol. In embodiments, the non-cannabis solvent is medium-chain triglycerides. In embodiments, the non-cannabis solvent is castor oil. In embodiments, the non-cannabis solvent is Kolliphor EL.

In embodiments, provided herein is a method of loading a non-crystalline cannabinoid oil into a plurality of colloidal silicon dioxide particles comprising internal storage cavities, said method comprising the steps of: (a) providing a non-crystalline cannabinoid oil; (b) heating the non-crystalline cannabinoid oil; (c) providing a plurality of colloidal silicon dioxide particles comprising internal storage cavities; and (d) contacting the non-crystalline cannabinoid oil with the plurality colloidal silicon dioxide particles comprising internal storage cavities, thereby loading substantially all of the non-crystalline cannabinoid oil into the colloidal silicon dioxide particles comprising internal storage cavities. In embodiments, the non-crystalline cannabinoid oil loaded into the colloidal silicon dioxide particles is in solid form. In embodiments, the non-crystalline cannabinoid oil is not diluted in a non-cannabis solvent. In embodiments, the non-cannabis solvent is an alcohol. In embodiments, the non-cannabis solvent is medium-chain triglycerides. In embodiments, the non-cannabis solvent is castor oil. In embodiments, non-cannabis solvent is Kolliphor EL.

In embodiments, provided herein is a method of making a Cannabinoid Product described herein, said method comprising the steps of: (a) providing a non-crystalline cannabinoid oil; (b) heating the non-crystalline cannabinoid oil; (c) providing a plurality of colloidal silicon dioxide particles comprising internal storage cavities; (d) mixing the non-crystalline cannabinoid oil and colloidal silicon dioxide particles comprising internal storage cavities; (e) adding a filler and disintegrant to the mixture of non-crystalline cannabinoid oil and colloidal silicon dioxide particles comprising internal storage cavities; and (f) adding a lubricant. In embodiments, the non-crystalline cannabinoid oil loaded into the colloidal silicon dioxide particles is in solid form. In embodiments, the non-crystalline cannabinoid oil is not diluted in a non-cannabis solvent. In embodiments, the non-cannabis solvent is an alcohol. In embodiments, the non-cannabis solvent is medium-chain triglycerides. In embodiments, the non-cannabis solvent is castor oil. In embodiments, the non-cannabis solvent is Kolliphor EL.

In embodiments, provided herein is a method of making a Cannabinoid Product described herein, said method comprising the steps of: (a) providing a non-crystalline cannabinoid oil; (b) heating the non-crystalline cannabinoid oil; (c) providing a plurality of colloidal silicon dioxide particles comprising internal storage cavities; (d) mixing the non-crystalline cannabinoid oil and colloidal silicon dioxide particles comprising internal storage cavities; and (e) adding a lubricant. In embodiments, the non-crystalline cannabinoid oil loaded into the colloidal silicon dioxide particles is in solid form. In embodiments, the non-crystalline cannabinoid oil is not diluted in a non-cannabis solvent. In embodiments, the non-cannabis solvent is an alcohol. In embodiments, the non-cannabis solvent is medium-chain triglycerides. In embodiments, the non-cannabis solvent is castor oil. In embodiments, the non-cannabis solvent is Kolliphor EL.

In embodiments, provided herein is a solid Cannabinoid Product comprising: (a) non-crystalline cannabinoid oil; and (b) colloidal silicon dioxide particles comprising internal storage cavities. Optionally, the non-crystalline cannabinoid oil is substantially loaded into the colloidal silicon dioxide storage particles. Optionally, the loaded non-crystalline cannabinoid oil to colloidal silicon dioxide particle ratio is about 0.5:1 to 3:1 by weight. Optionally, the non-crystalline cannabinoid oil comprises at least 80%, at least 90%, at least 95%, at least 99% cannabinoid by weight. Optionally, the non-crystalline cannabinoid oil is not diluted in a non-cannabis solvent. Optionally, the non-cannabis solvent is an alcohol. Optionally, the non-cannabis solvent is medium-chain triglycerides. Optionally, the non-cannabis solvent is castor oil. Optionally, the non-cannabis solvent is Kolliphor EL. Optionally, the loaded non-crystalline cannabinoid oil to colloidal silicon dioxide particle ratio is about 1:1 to 2:1 by weight. Optionally, the loaded non-crystalline cannabinoid oil to colloidal silicon dioxide particle ratio is about 1:5 by weight. Optionally, the composition comprises total non-cannabinoid solid ingredients; wherein said total non-cannabinoid solid ingredients are less than about 95%, about 90%, about 85%, about 80%, about 75%, about 70%, about 65%, about 60%, about 55%, about 50%, about 45%, about 40%, about 35%, about 30%, about 25%, or about 20% of the Cannabinoid Product by weight. Optionally, the non-crystalline cannabinoid oil is loaded onto the colloidal silicon dioxide particles in the absence of any other solid ingredients. Optionally, the Cannabinoid Product comprises less than 10%, 9%, 8%, 7%, 6%, 5%, 4%, 3%, 2%, 1% w/w cyclodextrin. Optionally, the Cannabinoid Product does not comprise cyclodextrin. Optionally, the Cannabinoid Product comprises less than 10%, 9%, 8%, 7%, 6%, 5%, 4%, 3%, 2%, 1% w/w emulsifier. Optionally, the Cannabinoid Product does not comprise an emulsifier. Optionally, the solid Cannabinoid Product has a hardness of from 50 Newtons (N) to 100 N. Optionally, at least 70% of the non-crystalline cannabinoid oil is loaded into the colloidal silicon dioxide storage particles.

In embodiments, provided herein is a solid Cannabinoid Product comprising: (a) non-crystalline cannabinoid oil; and (b) colloidal silicon dioxide particles comprising internal storage cavities. Optionally, the non-crystalline cannabinoid oil is substantially loaded into the colloidal silicon dioxide storage particles. Optionally, the non-crystalline cannabinoid oil content is at least 30% w/w of the colloidal silicon dioxide particle content. Optionally, the non-crystalline cannabinoid oil is not diluted in a non-cannabis solvent. Optionally, the non-cannabis solvent is an alcohol. Optionally, the non-cannabis solvent is medium-chain triglycerides. Optionally, the non-cannabis solvent is castor oil. Optionally, the non-cannabis solvent is Kolliphor EL. Optionally, the non-crystalline cannabinoid oil comprises at least 50% w/w of the colloidal silicon dioxide particles comprising internal storage cavities. Optionally, the non-crystalline cannabinoid oil comprises at least 70% w/w of the colloidal silicon dioxide particles comprising internal storage cavities. Optionally, the composition comprises total non-cannabinoid solid ingredients; wherein said total non-cannabinoid solid ingredients are less than about 95%, about 90%, about 85%, about 80%, about 75%, about 70%, about 65%, about 60%, about 55%, about 50%, about 45%, about 40%, about 35%, about 30%, about 25%, or about 20% of the Cannabinoid Product by weight. Optionally, the non-crystalline cannabinoid oil is loaded onto the colloidal silicon dioxide particles in the absence of any other solid ingredients. Optionally, the Cannabinoid Product comprises less than 10%, 9%, 8%, 7%, 6%, 5%, 4%, 3%, 2%, 1% w/w cyclodextrin. Optionally, the Cannabinoid Product does not comprise cyclodextrin. Optionally, the Cannabinoid Product comprises less than 10%, 9%, 8%, 7%, 6%, 5%, 4%, 3%, 2%, 1% w/w emulsifier. Optionally, the Cannabinoid Product does not comprise an emulsifier. Optionally, the solid Cannabinoid Product has a hardness of from 50 Newtons (N) to 100 N. Optionally, at least 70% of the non-crystalline cannabinoid oil is loaded into the colloidal silicon dioxide storage particles.

In embodiments, provided herein is a cannabinoid daily patient dose comprising: (a) non-crystalline cannabinoid oil comprising a cannabinoid; and (b) colloidal silicon dioxide particles comprising internal storage cavities. Optionally, the non-crystalline cannabinoid oil is substantially loaded into the colloidal silicon dioxide particles comprising internal storage cavities. Optionally, said daily dose comprises greater than 50 mg of the cannabinoid. Optionally, the daily dose is a pill. Optionally, the daily dose is a tablet. Optionally, the daily dose is a single pill. Optionally, the single pill comprises about 75 mg to about 150 mg cannabinoid. Optionally, the daily dose is at least two pills. Optionally, the daily dose is a single tablet. Optionally, the single tablet comprises about 75 mg to about 150 mg cannabinoid. Optionally, the daily dose is at least two tablets. Optionally, the daily dose comprises about 0.1 mg/kg/day to about 25 mg/kg/day of cannabinoid. Optionally, the daily dose comprises about 1 mg/kg/day of non-crystalline cannabinoid oil. Optionally, the single pill comprises about 50 mg to about 100 mg colloidal silicon dioxide particles comprising internal storage cavities. Optionally, the single tablet comprises about 50 mg to about 100 mg colloidal silicon dioxide particles comprising internal storage cavities. Optionally, the non-crystalline cannabinoid oil in the Cannabinoid Product is in solid form. Optionally, the non-crystalline cannabinoid oil is not diluted in a non-cannabis solvent. Optionally, the non-cannabis solvent is an alcohol. Optionally, the non-cannabis solvent is medium-chain triglycerides. Optionally, the non-cannabis solvent is castor oil. Optionally, the non-cannabis solvent is Kolliphor EL. Optionally, the composition comprises total non-cannabinoid solid ingredients; wherein said total non-cannabinoid solid ingredients are less than about 95%, about 90%, about 85%, about 80%, about 75%, about 70%, about 65%, about 60%, about 55%, about 50%, about 45%, about 40%, about 35%, about 30%, about 25%, or about 20% of the Cannabinoid Product by weight. Optionally, the non-crystalline cannabinoid oil is loaded onto the colloidal silicon dioxide particles in the absence of any other solid ingredients. Optionally, the Cannabinoid Product comprises less than 10%, 9%, 8%, 7%, 6%, 5%, 4%, 3%, 2%, 1% w/w cyclodextrin. Optionally, the Cannabinoid Product does not comprise cyclodextrin. Optionally, the Cannabinoid Product comprises less than 10%, 9%, 8%, 7%, 6%, 5%, 4%, 3%, 2%, 1% w/w emulsifier. Optionally, the Cannabinoid Product does not comprise an emulsifier.

In embodiments, provided herein is a solid Cannabinoid Product comprising: (a) non-crystalline cannabinoid oil; and (b) colloidal silicon dioxide particles. Optionally, the non-crystalline cannabinoid oil is substantially loaded into the colloidal silicon dioxide storage particles. Optionally, the product exhibits a linear dissolution rate as measured in 1% Tween 80 media, Fasted State Simulated Intestinal Fluid (FaSSIF) media, and/or Fed State Simulated Intestinal Fluid (FeSSIF) media. Optionally, the dissolution rate remains linear throughout 5-60 minutes after contacting the product with the 1% tween 80 media, FaSSIF media, and/or FeSSIF media. Optionally, the dissolution rate remains linear throughout a first phase, a second phase, and third phase after contacting the product with the 1% tween 80 media, FaSSIF media, and/or FeSSIF media. Optionally, the first phase comprises a period of 5-14 minutes after contacting the product with the 1% tween 80 media, FaSSIF media, and/or FeSSIF media, wherein the second phase comprises a period of 15-34 minutes after contacting the product with the 1% tween 80 media, FaSSIF media, and/or FeSSIF media, and wherein the third phase comprises a period of 35-60 minutes after contacting the product with the 1% tween 80 media, FaSSIF media, and/or FeSSIF media. Optionally, the linear dissolution rate comprises an R² value not less than about 0.90. Optionally, the Cannabinoid Product exhibits a linear absorption rate for at least 20 minutes after administration. Optionally, the Cannabinoid Product exhibits a linear absorption rate for at least 40 minutes after administration. Optionally, the Cannabinoid Product exhibits a linear absorption rate for at least 1 hour after administration. Optionally, the non-crystalline cannabinoid oil in the Cannabinoid Product is in solid form. Optionally, the non-crystalline cannabinoid oil is not diluted in a non-cannabis solvent. Optionally, the non-cannabis solvent is an alcohol. Optionally, the non-cannabis solvent is medium-chain triglycerides. Optionally, the non-cannabis solvent is castor oil. Optionally, the non-cannabis solvent is Kolliphor EL. Optionally, the composition comprises total non-cannabinoid solid ingredients; wherein said total non-cannabinoid solid ingredients are less than about 95%, about 90%, about 85%, about 80%, about 75%, about 70%, about 65%, about 60%, about 55%, about 50%, about 45%, about 40%, about 35%, about 30%, about 25%, or about 20% of the Cannabinoid Product by weight. Optionally, the non-crystalline cannabinoid oil is loaded onto the colloidal silicon dioxide particles in the absence of any other solid ingredients. Optionally, the Cannabinoid Product comprises less than 10%, 9%, 8%, 7%, 6%, 5%, 4%, 3%, 2%, 1% w/w cyclodextrin. Optionally, the Cannabinoid Product does not comprise cyclodextrin. Optionally, the Cannabinoid Product comprises less than 10%, 9%, 8%, 7%, 6%, 5%, 4%, 3%, 2%, 1% w/w emulsifier. Optionally, the Cannabinoid Product does not comprise an emulsifier. Optionally, the solid Cannabinoid Product has a hardness of from 50 Newtons (N) to 100 N. Optionally, at least 70% of the non-crystalline cannabinoid oil is loaded into the colloidal silicon dioxide storage particles.

In embodiments, provided herein is a method of loading into a non-crystalline cannabinoid oil into a plurality of colloidal silicon dioxide particles comprising internal storage cavities, said method comprising the steps of: (a) providing a non-crystalline cannabinoid oil; (b) heating the non-crystalline cannabinoid oil; (c) providing a plurality of colloidal silicon dioxide particles comprising internal storage cavities; and (d) contacting the non-crystalline cannabinoid oil with the plurality colloidal silicon dioxide particles comprising internal storage cavities, thereby loading substantially all of the non-crystalline cannabinoid oil into the colloidal silicon dioxide particles comprising internal storage cavities. Optionally, the non-crystalline cannabinoid oil loaded into the colloidal silicon dioxide particles is in solid form. Optionally, the non-crystalline cannabinoid oil is not diluted in a non-cannabis solvent. Optionally, the non-cannabis solvent is an alcohol. Optionally, the non-cannabis solvent is medium-chain triglycerides. Optionally, the non-cannabis solvent is castor oil. Optionally, the non-cannabis solvent is Kolliphor EL.

In embodiments, provided herein is a method of making a solid Cannabinoid Product, said method comprising the steps of: (a) providing a non-crystalline cannabinoid oil; (b) heating the non-crystalline cannabinoid oil; (c) providing a plurality of colloidal silicon dioxide particles comprising internal storage cavities; (d) mixing the non-crystalline cannabinoid oil and colloidal silicon dioxide particles comprising internal storage cavities; (e) adding a filler and disintegrant to the mixture of non-crystalline cannabinoid oil and colloidal silicon dioxide particles comprising internal storage cavities; and (f) adding a lubricant. Optionally, the non-crystalline cannabinoid oil loaded into the colloidal silicon dioxide particles is in solid form. Optionally, the non-crystalline cannabinoid oil is not diluted in a non-cannabis solvent. Optionally, the non-cannabis solvent is an alcohol. Optionally, the non-cannabis solvent is medium-chain triglycerides. Optionally, the non-cannabis solvent is castor oil. Optionally, the non-cannabis solvent is Kolliphor EL. Optionally, the solid Cannabinoid Product further comprises total non-cannabinoid solid ingredients; wherein said total non-cannabinoid solid ingredients are less than about 95%, about 90%, about 85%, about 80%, about 75%, about 70%, about 65%, about 60%, about 55%, about 50%, about 45%, about 40%, about 35%, about 30%, about 25%, or about 20% of the Cannabinoid Product by weight. Optionally, the non-crystalline cannabinoid oil is loaded onto the colloidal silicon dioxide particles in the absence of any other solid ingredients. Optionally, the solid Cannabinoid Product comprises less than 10%, 9%, 8%, 7%, 6%, 5%, 4%, 3%, 2%, 1% w/w cyclodextrin. Optionally, the solid Cannabinoid Product does not comprise cyclodextrin. Optionally, the solid Cannabinoid Product comprises less than 10%, 9%, 8%, 7%, 6%, 5%, 4%, 3%, 2%, 1% w/w emulsifier. Optionally, the solid Cannabinoid Product does not comprise an emulsifier. Optionally, the solid Cannabinoid Product has a hardness of from 50 Newtons (N) to 100 N. Optionally, at least 70% of the non-crystalline cannabinoid oil is loaded into the colloidal silicon dioxide storage particles.

In embodiments, provided herein is a method of making a Cannabinoid Product, said method comprising the steps of: (a) providing a non-crystalline cannabinoid oil; (b) heating the non-crystalline cannabinoid oil; (c) providing a plurality of colloidal silicon dioxide particles comprising internal storage cavities; (d) mixing the non-crystalline cannabinoid oil and colloidal silicon dioxide particles comprising internal storage cavities; and (e) adding a lubricant. Optionally, the non-crystalline cannabinoid oil loaded into the colloidal silicon dioxide particles is in solid form. Optionally, the non-crystalline cannabinoid oil is not diluted in a non-cannabis solvent. Optionally, the non-cannabis solvent is an alcohol. Optionally, the non-cannabis solvent is medium-chain triglycerides. Optionally, the non-cannabis solvent is castor oil. Optionally, the non-cannabis solvent is Kolliphor EL. Optionally, the solid Cannabinoid Product further comprises total non-cannabinoid solid ingredients; wherein said total non-cannabinoid solid ingredients are less than about 95%, about 90%, about 85%, about 80%, about 75%, about 70%, about 65%, about 60%, about 55%, about 50%, about 45%, about 40%, about 35%, about 30%, about 25%, or about 20% of the Cannabinoid Product by weight. Optionally, the non-crystalline cannabinoid oil is loaded onto the colloidal silicon dioxide particles in the absence of any other solid ingredients. Optionally, the solid Cannabinoid Product comprises less than 10%, 9%, 8%, 7%, 6%, 5%, 4%, 3%, 2%, 1% w/w cyclodextrin. Optionally, the solid Cannabinoid Product does not comprise cyclodextrin. Optionally, the solid Cannabinoid Product comprises less than 10%, 9%, 8%, 7%, 6%, 5%, 4%, 3%, 2%, 1% w/w emulsifier. Optionally, the solid Cannabinoid Product does not comprise an emulsifier. Optionally, the solid Cannabinoid Product has a hardness of from 50 Newtons (N) to 100 N. Optionally, at least 70% of the non-crystalline cannabinoid oil is loaded into the colloidal silicon dioxide storage particles.

### BRIEF DESCRIPTION OF THE DRAWINGS

FIG. 1A shows a non-crystalline cannabinoid oil. The composition does not contain colloidal silicon dioxide particles, and is therefore a viscous liquid. FIG. 1B shows a composition, according to the present disclosure, containing a non-crystalline cannabinoid and colloidal silicon dioxide particles. This composition is in solid, powder form, and stable.

FIG. 2 shows an image of the Torpac ProFiller® 3700 capsule filler used to fill the capsules of Examples 1, 2, 5, and 6-9.

FIG. 3 shows a scanning electron micrograph of an example of colloidal silicon dioxide particles of the disclosure. (Technical Information 1414 AEROPERL® 300 EVONIK.).

FIG. 4 shows an image of a machine used to determine the hardness of a tablet.

FIG. 5 shows a graph comparing the dissolution rate over time of cannabinoid test capsules produced with and without heating non-crystalline cannabinoid oil prior to loading with colloidal silica in either FeSSIF media or FaSSIF media. Test capsules were produced according to the methods of Example 7. Test capsules produced with heated, non-crystalline, non-crystalline cannabinoid oil exhibited higher dissolution rates in both FeSSIF and FaSSIF (compare light and dark squares and light and dark circles).

FIG. 6 shows a graph comparing the dissolution rate over time of test capsules produced with varying non-crystalline cannabinoid: colloidal silica ratios (ranging 1.5:1 to 0.5:1) non-crystalline cannabinoid oil in FaSSIF media. Test capsules were produced according to the methods of Example 8.

FIG. 7 shows a graph comparing the dissolution rate over time of test capsules produced with varying non-crystalline cannabinoid: colloidal silica ratios (ranging 1.5:1 to 0.5:1) non-crystalline cannabinoid oil in 1% Tween 80. Test capsules were produced according to the methods of Example 8.

FIG. 8 shows a graph comparing the dissolution rate over time of the Cannabinoid Products of the present disclosure against an older-generation formulation with a 1:1, unheated cannabinoid: colloidal silica formulation in 1% Tween 80 media. Cannabinoid Products of the present disclosure exhibited linear dissolution, while the older-generation formulation exhibited highly logarithmic dissolution.

### DETAILED DESCRIPTION

### I. Definitions

The term “a” or “an” refers to one or more of that entity, i.e., can refer to a plural referent. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Throughout this application, the term “about” is used to indicate that a value includes the inherent variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

Herein, the terms “comprise”, or variations such as “comprises” or “comprising”, will be understood to imply the inclusion of a stated element or integer or group of elements or integers but not the exclusion of any other element or integer or group of elements or integers.

The term “substantially pure” refers to a cannabinoid having a chromatographic purity of greater than about 95% by weight, greater than about 96% by weight, greater than about 97% by weight, greater than about 98% by weight, or greater than about 99% by weight.

Various concentration expressions, including volume concentrations, weight concentrations, and mass concentrations, are utilized to describe the percentage of a component in a solution. Volume concentration has units of % v/v, where v/v is volume per volume. If a solution contains 5% v/v of a component, 5 mL of the component is in a total solution of 100 mL, for example. Weight concentration of a solution is expressed as % w/w, where w/w is weight per weight. If a solution contains 30% w/w of sodium chloride, an example solution contains 30 g of sodium chloride and 70 g of other components of the solution, such as the solvent. Mass concentration of a solution is expressed as % w/v, where w/v is weight per volume. If 1 g of sodium chloride is dissolved in a solution with a total volume of 100 mL, a 1% w/v sodium chloride solution has been made.

The term “Cannabis plant part” refers to any part of a Cannabis plant including but not limited to the embryo, shoot, root, stem, seed, stipule, leaf, petal, flower, inflorescence, bud, ovule, bract, trichome, branch, petiole, internode, bark, pubescence, tiller, rhizome, frond, blade, pollen, stamen, mesocarp, epicarp, endosperm, spermoderm, and disk.

The term “Cannabinoid Product” refers to a composition comprising chemical constituents of Cannabis plants. In embodiments, the Cannabinoid Product is a solid, (e.g., a powder).

The term “cannabinoid oil,” used interchangeably with “hemp oil,” refers to a composition produced by extracting cannabinoids from a Cannabis plant or Cannabis plant part(s). In some embodiments, the cannabis oil is a heated such that it is a non-crystalline cannabinoid oil.

The term “cannabinoid distillate” refers to a liquid cannabinoid oil composition that has been distilled. Distillates may contain more than one cannabinoid and may contain other cannabis compounds, such as terpenes. In some embodiments, a cannabinoid distillate and/or “non-crystalline cannabinoid oil” is substantially free of waxes and chlorophylls, but remains in liquid, oily form.

The term “cannabinoid isolate” refers to a composition comprising at least 95% of a single cannabinoid that is typically solid at room temperature. Cannabinoid isolate is typically in crystalline form at room temperature, or rapidly crystallizes when exposed to nucleation factors, such as other powders.

The term “substantially disintegrates” refers to a level of disintegration amounting to disintegration of at least about 50%, at least about 60%, at least about 70%, at least about 80%, at least about 90%, or about 100% disintegration. The term “disintegration” is distinguished from the term “dissolution,” in that “disintegration” refers to the breaking up of or loss of structural cohesion of e.g. the constituent particles comprising a tablet, whereas “dissolution” refers to the solubilization of a solid in a liquid (e.g., the solubilization of a Cannabinoid Product in solvents or gastric fluids). Disintegration may be measured by recording the amount of time it takes for a tablet of a particular size to disintegrate. Alternatively, the rate of disintegration can be measured using various in vitro test methods, for example the United States Pharmacopeia (USP)<701> Disintegration Test. USP <701> is incorporated by reference herein in its entirety. Dissolution may be measured using a USP Type II paddle dissolution apparatus (paddles at 100 rpm) or a USP Type I basket apparatus at a temperature of about 37° C. at a particular time. The percentage of Cannabinoid Product dissolved at 60 minutes is referred to as the “Percentage Dissolved at 60 minutes.” In embodiments, dissolution measurements use a high performance liquid chromatography, ultraviolet visible spectroscopy, and fiber optic dissolution may be used to measure dissolution.

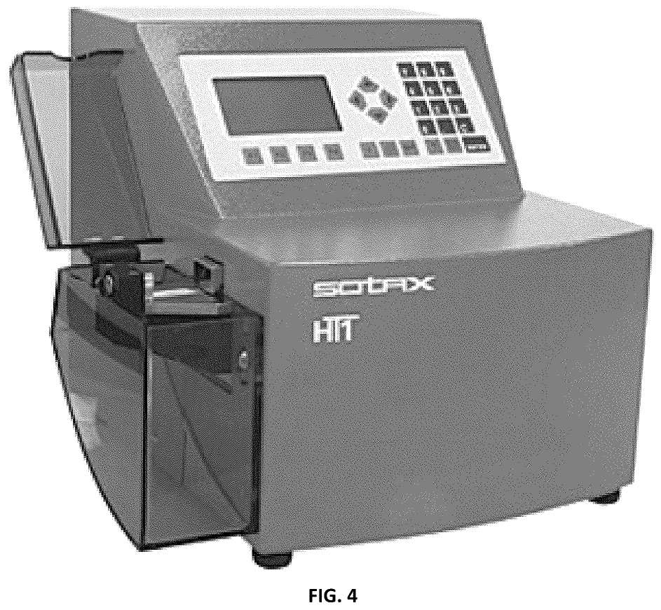

The term “hardness” refers to a tablet's “breaking force,” which is the force required to cause the tablet to fail in a specific plane. A tablet's “breaking force” is measured by placing the tablet between two platens, one of which moves to apply sufficient force to cause fracture. For round tablets, fracture occurs in the plane of loading, which occurs across the tablet's diameter. USP <1217> describes the tablet breaking force and is incorporated by reference herein in its entirety. FIG. 4 provides an example schematic of a machine used to determine the hardness of a tablet.

The term “compression force” refers to the force exerted between the upper and lower punches when compressing a Cannabinoid Product into a tablet as determined by a load cell transducer that converts the force into electrical signal using a strain gauge. The compression force may be measured in Newtons (N).

The terms “orally disintegrating tablet,” “orally dispersing tablet,” or “ODT” refer to a solid dosage form, which disintegrates rapidly in the oral cavity of a patient after administration, without chewing.

Value ranges within this disclosure may indicate that they are intended to include “all ranges and subranges therebetween” or similar language. This should be interpreted to include the ranges between any two recited values (including values between recited values), or ranges that are above or below the recited value. For example, a recited range of 1%, 2%, 3%, or 4% in the disclosure is intended to provided basis for claims reciting i) values “between” 2% and 4%, ii) “between” 2.5% and 3%, iii) “less than” 3%, and iv) “greater than” 4%.

### II. Cannabis

Cannabinoid Products contain chemical constituents from Cannabis plants. Cannabis is a genus of flowering plants that includes three different species, Cannabis sativa, Cannabis indica and Cannabis ruderalis. There are 483 identifiable chemical constituents known to exist in the cannabis plant (Rudolf Brenneisen (2007) in Marijuana and the Cannabinoids, ElSohly, ed.; incorporated herein by reference), including at least 85 different cannabinoids and over 120 terpenes (El-Alfy, Abir T, et al. (2010) Pharmacology Biochemistry and Behavior 95 (4): 434-42; incorporated herein by reference). The two most well-known cannabinoids produced by Cannabis plants are tetrahydrocannabinol (THC) and cannabidiol (CBD).

### II-A. Cannabinoids

Cannabinoids are a unique family of terpeno-phenolic compounds produced by Cannabis plants. Typical cannabinoids isolated from Cannabis plants include, but are not limited to, Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), Tetrahydrocannabinolic acid A (THCA-A), Tetrahydrocannabinolic acid B (THCA-B), Tetrahydrocannabinolic Acid (THCA), Tetrahydrocannabinol (THC), Tetrahydrocannabinolic acid C4 (THCA-C4), Tetrahydrocannabinol C4 (THC-C4), Tetrahydrocannabivarinic acid (THCVA), Tetrahydrocannabivarin (THCV), Tetrahydrocannabinolic acid-C4 (THCA-C4), Tetrahydrocannabinol-C1 (THC-C1), A7-cis-iso-tetrahydrocannabivarin, Δ8-tetrahydrocannabinolic acid (Δ8-THCA), Cannabivarinodiolic (CBNDVA), Cannabivarinodiol (CBNDV), Δ8-tetrahydrocannabinol (Δ8-THC), Δ9-tetrahydrocannabinol (Δ9-THC), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabivarinselsoin (CBEV), Cannabivarinselsoinic Acid (CBEVA), Cannabielsoic Acid (CBEA), Cannabielvarinsoin (CBLV), Cannabielvarinsoinic Acid (CBLVA), Cannabinolic acid (CBNA), Cannabinol (CBN), Cannabivarinic Acid (CBNVA), Cannabinol methylether (CBNM), Cannabinol-C4 (CBN-C4), Cannabivarin (CBV), Cannabino-C2 (CBN-C2), Cannabiorcol (CBN-C1), Cannabinodiol (CBND), Cannabinodiolic Acid (CBNDA), Cannabinodivarin (CBDV), Cannabitriol (CBT), 10-Ethoxy-9-hydroxy-46a-tetrahydrocannabinol, 8,9-Dihydroxy-46a (10a)-tetrahydrocannabinol (8,9-Di-OH-CBT-C5), Cannabitriolvarin (CBTV), Ethoxy-cannabitriolvarin (CBTVE), Dehydrocannabifuran (DCBF), Cannbifuran (CBF), Cannabichromanon (CBCN), Cannabicitran (CBT), 10-Oxo-Δ6a(10a)-tetrahydrocannabinol (OTHC), Δ9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, and Dodeca-2E,4E-dienoic acid isobutylamide. See Holley et al. (1975) J. Pharm. Sci. 64:892-894 and De Zeeuw et al. (9172) Science 175:778-779, each of which is herein incorporated by reference in its entirety for all purposes.

Most cannabinoids exist in two forms, as acids and in neutral (decarboxylated) forms. The acid form is designated by an “A” at the end of its acronym (i.e. THCA). The phytocannabinoids are synthesized in the plant as acid forms, and while some decarboxylation does occur in the plant, it increases significantly post-harvest and the kinetics increase at high temperatures. (Sanchez and Verpoorte 2008). Cannabinoids in their acid forms (those ending in “-A”) can be converted to their non-acidic forms through a process called decarboxylation. While some decarboxylation (e.g., neutralization) of cannabinoids does occur in the plant, production of the neutral forms increases significantly post-harvest. (Sanchez and Verpoorte (2008) Plant Cell Physiol. December: 49 (12)). Full decarboxylation of phytocannabinoids can be catalyzed by post-cultivation heating cannabis plant material or extracted cannabinoids (e.g., by combustion, vaporization, or baking in an oven).

In order to find the total amount of non-acidic cannabinoid, the total measured content of acid cannabinoid variants forms should be adjusted to account for the loss of the carboxyl group. In some embodiments, this adjustment can be made by multiplying the molar content of the acidic cannabinoid forms by the molecular weight of the corresponding decarboxylated cannabinoid. Other shorthand conversions are also available for quickly converting acidic cannabinoid content to active cannabinoid content.

For example, in some embodiments, THCA can be converted to active THC using the formula: THCA×0.877=THC. When using this approach, the maximum THC for the sample is: THCmax=(THCA×0.877)+THC. This method has been validated according to the principles of the International Conference on Harmonization. Similarly, CBDA can be converted to active CBD and the yield is determined using the yield formula: CBDA×0.877=CBD. Also, the maximum amount of CBD yielded, i.e. max CBD for the sample is: CBDmax=(CBDA×0.877)+CBD. Additionally, CBGA can be converted to active CBG by multiplying CBGA by 0.878 (CBGmax=(CBGA×0.878)+CBG).

In some embodiments, provided herein are Cannabinoid Products containing cannabinoids. In some embodiments, the Cannabinoid Products contain a cannabinoid selected from the group consisting of: cannabidiol (CBD), tetrahydrocannabinol (THC), delta 8 tetrahydrocannabinol (D8 THC or A8 THC), tetrahydrocannabivarin (THCV), cannabidiolic acid (CBDA), cannabidivarin (CBDV), tetrahydrocannabinolic acid (THCA), cannabigerol (CBG), cannabigerolic acid (CBGA), cannabinol (CBN), cannabinolic acid (CBNA), cannabidivarinic acid (CBDVA), cannabichromenic acid (CBCA), and cannabichromene (CBC).

Brief descriptions and chemical structures of the aforementioned cannabinoids are provided below.

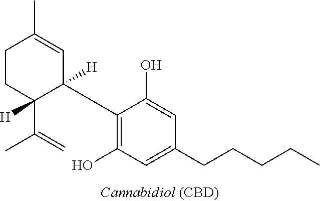 [SMILES: C=C(C)[C@@H]1CCC(C)=C[C@H]1c1c(O)cc(CCCCC)cc1O]

CBD is a cannabinoid found in Cannabis shown to display sedative effects in animal tests (Pickens, (1981) Br. J. Pharmacol. 72 (4): 649-56). Some research, however, indicates that CBD can increase alertness, and attenuates the memory-impairing effect of THC. (Nicholson et al., June (2004) J Clin Psychopharmacol 24 (3): 305-13; Morgan et al., (2010) The British Journal of Psychiatry, 197:258-290). CBD may also decrease the rate of THC clearance from the body, perhaps by interfering with the metabolism of THC in the liver. Medically, CBD has been shown to relieve convulsion, inflammation, anxiety, and nausea, as well as inhibit cancer cell growth (Mechoulam, et al., 2007, Chemistry & Biodiversity 4 (8): 1678-1692), for example reducing growth and invasiveness of aggressive human breast cancer cells (McAllister et al., 2007, Mol. Cancer Ther. 6 (11): 2921-7) Recent studies have also shown CBD to be as effective as an atypical antipsychotic for treating schizophrenia (Zuardi et al., 2006, Braz. J. Med. Biol. Res. 39 (4): 421-429.), and studies also suggests that CBD may relieve symptoms of dystonia (Consroe, 1986, The International journal of neuroscience 30 (4): 277-282).

Cannabis produces cannabidiolic acid (CBDA) through the same metabolic pathway as THC, until the last step, where CBDA synthase performs catalysis instead of THCA synthase. See Marks et al. (2009) Journal of Experimental Botany 60 (13): 3715-3726 and Meijer et al. I, II, III, and IV.

Non-limiting examples of CBD variants include CBD-C5, CBDM-C5, CBD-C4, CBDV-C3, CBD-C1, CBDA-C5, and CBDVA-C3.

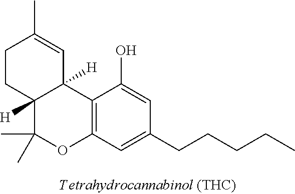 [SMILES: CCCCCc1cc(O)c2c(c1)OC(C)(C)[C@@H]1CCC(C)=C[C@@H]21]

Unless indicated otherwise, as used herein, THC refers to delta-9-tetrahydrocannabinol (Δ9-THC) (structure above). THC is the principal psychoactive constituent (or cannabinoid) of the Cannabis plant. THC has mild to moderate analgesic effects, and Cannabis can be used to treat pain by altering transmitter release on dorsal root ganglion of the spinal cord and in the periaqueductal gray. Other effects include relaxation, alteration of visual, auditory, and olfactory senses, fatigue, and appetite stimulation. THC has marked antiemetic properties, and may also reduce aggression in certain subjects (Hoaken (2003) Addictive Behaviors 28:1533-1554).

The pharmacological actions of THC result from its partial agonist activity at the cannabinoid receptor CB1, located mainly in the central nervous system, and the CB2 receptor, mainly expressed in cells of the immune system (Pertwee, (2006) International Journal of Obesity 30: S13-S18.) It is also suggested that THC has an anticholinesterase action, which may implicate it as a potential treatment for Alzheimer's and Myasthenia gravis (Eubanks et al., (2006) Molecular Pharmaceutics 3 (6): 773-7).

In the cannabis plant, THC occurs mainly as tetrahydrocannabinolic acid (THCA, 2-COOH-THC). Geranyl pyrophosphate and olivetolic acid react via an enzyme-catalyzed reaction to produce cannabigerolic acid, which is cyclized by the enzyme THC acid synthase to give THCA. Over time, or when heated, THCA is decarboxylated producing THC.

Non-limiting examples of THC variants include Δ9-THC-C5, Δ9-THC-C4, Δ9-THCV-C3, Δ9-THCO-C1, Δ9-THCA-C5 A, Δ9-THCA-C5 B, Δ9-THCA-C4 A, Δ9-THCA-C4 B, Δ9-THCVA-C3 A, Δ9-THCOA-C1 A, Δ9-THCOA-C1 B, Δ8-THC-C5, Δ8-THCA-C5 A, (−)-cis-49-THC-C5. The structure of Δ8-THC is below.

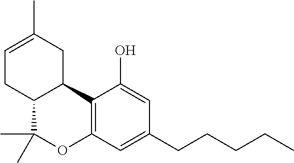 [SMILES: CCCCCc1cc(O)c2c(c1)OC(C)(C)[C@@H]1CC=C(C)C[C@@H]21]

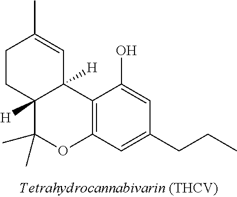 [SMILES: CCCc1cc(O)c2c(c1)OC(C)(C)[C@@H]1CCC(C)=C[C@@H]21]

THCV is a propyl analogue of tetrahydrocannabinol. Although THCV possesses an almost identical structure to Δ9-THC (varying only by the length of its lipophilic alkyl chain), it has different molecular targets and pharmacological profile. Compared to THC which demonstrates its effects through weak partial agonist activity of both endocannabinoid receptors Cannabinoid-1 (CB1R) and Cannabinoid-2 (CB2R), THCV acts as a CB1 antagonist and a partial agonist of CB2. THCV has been reported to activate 5HT1A receptors to produce an antipsychotic effect that has therapeutic potential for ameliorating some of the negative, cognitive and positive symptoms of schizophrenia (Br J Pharmacol. 2015 March; 172 (5): 1305-1318.) THCV has also shown antiepileptiform and anticonvulsant properties that suggest possible therapeutic application in the treatment of pathophysiologic hyperexcitability states such as untreatable epilepsy (Epilepsia. 2010 August; 51 (8): 1522-32.).

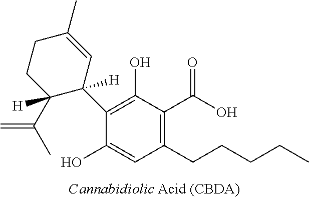 [SMILES: C=C(C)[C@@H]1CCC(C)=C[C@H]1c1c(O)cc(CCCCC)c(C(=O)O)c1O]

CBDA is most abundant in the glandular trichomes on Cannabis buds. CBDA is the chemical precursor to CBD. CBDA has been shown to exert therapeutic effects, including antiemetic, anti-inflammatory, anxiolytic, and antidepressant effects. (Behav Pharmacol. 2020 September; 31 (6): 591-596.) An in vivo rodent study showed that CBDA reduces thermal pain sensitivity in a model of Rett syndrome. (Neuroscience. 2021 Jan. 15; 453, 113-123.)

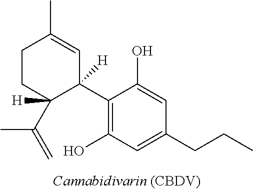 [SMILES: C=C(C)[C@@H]1CCC(C)=C[C@H]1c1c(O)cc(CCC)cc1O]

CBDV is a non-psychoactive cannabinoid found in Cannabis. It is a homolog of CBD with the side-chain shortened by two methylene bridges (CH₂ units). CBDV has demonstrated anticonvulsant activity in animal models and in human clinical trials. (Br J Pharmacol. 2013 October; 170 (3): 679-92.) CBDV also acts as a functional partial agonist on dopamine D2-like receptors in vivo, supporting its therapeutic use in neurological disorders. (Biochemical and Biophysical Research Communications. 2020; 533 (4): 1366-1370.).

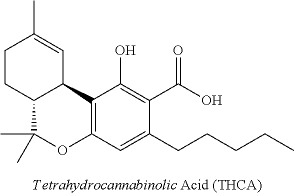 [SMILES: CCCCCc1cc2c(c(O)c1C(=O)O)[C@@H]1C=C(C)CC[C@H]1C(C)(C)O2]

THCA is found in variable quantities in fresh, undried Cannabis, but is progressively decarboxylated to THC with drying and heating. THCA is produced from cannabigerolic acid by THCA synthase. THCA has been shown to have anti-inflammatory properties. (Biol Pharm Bull. 2011; 34 (5): 774-8.).

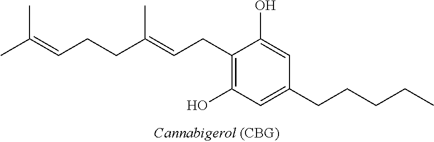 [SMILES: CCCCCc1cc(O)c(C/C=C(\C)CCC=C(C)C)c(O)c1]

CBG is a non-psychoactive cannabinoid found in the Cannabis plants. CBG has been found to act as a high affinity a2-adrenergic receptor agonist, moderate affinity 5-HT1A receptor antagonist, and low affinity CB1 receptor antagonist. It also binds to the CB2 receptor. Cannabigerol has also been shown to reduce depression in animal models (US Patent Application Publication No. 2008-0031977). In particular, CBG has been shown to have significant potential applications in the treatment of glaucoma, depression, Huntington's disease, MRSA, cachexia, and cancer (Craig et al. 1984, Experimental eye research 39 (3): 251-259; U.S. Pat. No. 8,481,085; Valdeolivas et al. 2015, Neurotherapeutics January 12 (1): 185-99; Appendino G et al., 2008, J. Nat Prod. August: 71 (8): 1427-30; Borrelli F et al. 2013, Biochem Pharmacol Mayl: 85 (9): 1306-16; Borrelli F. et al. 2014, Carcinogenesis December: 35 (12): 2787-97) Non-limiting examples of CBG variants include (E)-CBG-C5, (E)-CBGM-C5 A, (Z)-CBGA-C5 A, (E)-CBGV-C3, (E)-CBGA-C5 A, (E)-CBGAM-C5 A, and (E)-CBGVA-C3 A.

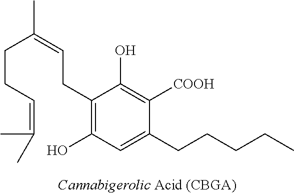 [SMILES: CCCCCc1cc(O)c(C/C=C(/C)CCC=C(C)C)c(O)c1C(=O)O]

CBGA is the acidic form of CBG. CBGA is synthesized from olivetolic acid and geranyl diphosphate. CBGA is a precursor to THCA, CBDA, and CBCA. CBGA has been found to inhibit aldose reductase activity, suggesting that it may have therapeutic value for treatment of complications of diabetes. (Fitoterapia. 2018 June; 127:101-108.).

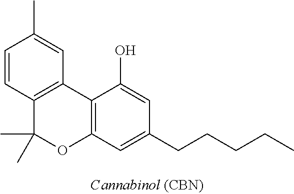 [SMILES: CCCCCc1cc(O)c2c(c1)OC(C)(C)c1ccc(C)cc1-2]

CBN is a mildly psychoactive cannabinoid found in trace amounts in Cannabis. CBN is a metabolite of THC. CBN has been shown to have significant applications in the treatment of anxiety disorder, insomnia, inflammation, convulsions, and bacterial infections. (Herring et al. Journal of Pharmacology and Experimental Therapeutics December 1999, 291 (3) 1156-1163.) CBN may be produced by aromatizing THC.

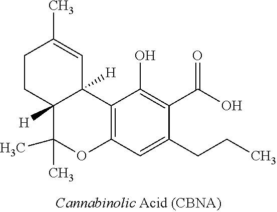 [SMILES: CCCc1cc2c(c(O)c1C(=O)O)[C@@H]1C=C(C)CC[C@H]1C(C)(C)O2]

CBNA is found in trace amounts in Cannabis. CBNA is produced from THCA after exposure to ultraviolet light. CBNA has therapeutic benefit for treatment of insomnia, convulsions, and chronic pain.

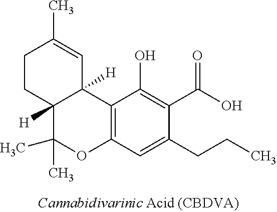 [SMILES: CCCc1cc2c(c(O)c1C(=O)O)[C@@H]1C=C(C)CC[C@H]1C(C)(C)O2]

CBDVA is the acidic form of CBDV. Extracts containing CBDVA exhibit antioxidant and antimicrobial activity against methicillin-resistant strains of Staphylococcus aureus. (Phytother Res. 2021 February; 35 (2): 1099-1112.).

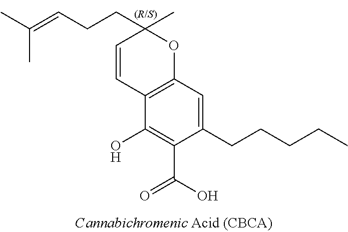 [SMILES: CCCCCc1cc2c(c(O)c1C(=O)O)C=CC(C)(CCC=C(C)C)O2]

CBCA is the acidic form of CBC. CBCA is produced from CBGA. Geranyl pyrophosphate and olivetolic acid combine to produce CBGA, which is cyclized by the enzyme CBCA synthase to form CBCA. In vitro studies show that CBCA has more potent bactericidal activity than vancomycin, the currently recommended treatment for methicillin-resistant Staphylococcus aureus infections. (Antibiotics 2020, 9 (8), 523).

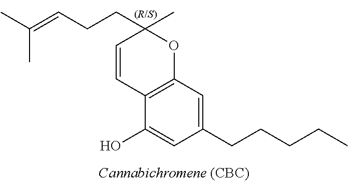 [SMILES: CCCCCc1cc(O)c2c(c1)OC(C)(CCC=C(C)C)C=C2]

CBC is produced from decarboxylation of CBCA. CBC is an agonist of TRPA1 and less potently an agonist of TRPV3 and TRPV4. CBC inhibits nitric oxide production in macrophages and ameliorates murine colitis. (Br J Pharmacol. 2013 May; 169 (1): 213-229; Acta Physiol (Oxf). 2012 February; 204 (2): 255-66.).

### II-B. Additional Components of Cannabis

In embodiments, in addition to containing cannabinoids, the Cannabinoid Products described herein may comprise additional components of Cannabis. Examples of additional components of Cannabis include non-specific lipid-soluble material or “ballast” e.g. waxes, wax esters and glycerides, unsaturated fatty acid residues, terpenes, chlorophyll, carotenes, flavonoids, pigments, sugars, cellulose compounds, and minerals.

Cannabis produces over 120 different terpenes (Russo (2011) British Journal of Pharmacology, 163:1344-1364). Within the context and verbiage of this document the terms ‘terpenoid’ and ‘terpene’ are used interchangeably. In some embodiments, the present disclosure provides compositions comprising one or more terpenes or terpenoids.

In addition to many circulatory and muscular effects, some terpenes interact with neurological receptors. A few terpenes produced by cannabis plants also bind weakly to cannabinoid receptors. Some terpenes can alter the permeability of cell membranes and allow in either more or less THC, while other terpenes can affect serotonin and dopamine chemistry as neurotransmitters. Terpenoids are lipophilic, and can interact with lipid membranes, ion channels, a variety of different receptors (including both G-protein coupled odorant and neurotransmitter receptors), and enzymes. Some are capable of absorption through human skin and passing the blood brain barrier.

Terpenoids are mainly synthesized in two metabolic pathways: mevalonic acid pathway (a.k.a. HMG-COA reductase pathway, which takes place in the cytosol) and MEP/DOXP pathway (a.k.a. The 2-C-methyl-D-erythritol 4-phosphate/1-deoxy-D-xylulose 5-phosphate pathway, non-mevalonate pathway, or mevalonic acid-independent pathway, which takes place in plastids). Geranyl pyrophosphate (GPP), which is used by cannabis plants to produce cannabinoids, is formed by condensation of dimethylallyl pyrophosphate (DMAPP) and isopentenyl pyrophosphate (IPP) via the catalysis of GPP synthase. Alternatively, DMAPP and IPP are ligated by FPP synthase to produce farnesyl pyrophosphate (FPP), which can be used to produce sesquiterpenoids. Geranyl pyrophosphate (GPP) can also be converted into monoterpenoids by limonene synthase.

Terpenes are derived biosynthetically from units of isoprene, which have the molecular formula C5H8. The basic molecular formulae of terpenes are multiples of (C5H8) n where n is the number of linked isoprene units. The isoprene units may be linked together “head to tail” to form linear chains or they may be arranged to form rings.

Within the context of this disclosure, the term “terpene” includes Hemiterpenes, Monoterpenols, Terpene esters, Diterpenes, Monoterpenes, Polyterpenes, Triterpenes, Tetraterpenes, Terpenoid oxides, Sesterterpenes, Sesquiterpenes, Norisoprenoids, as well as their isomers, enantiomers, or derivatives. Within the context of this disclosure, the term terpene includes the α-(alpha), β-(beta), γ-(gamma), oxo-, isomers, or any combinations thereof.

In some embodiments, the present disclosure provides Cannabinoid Products containing terpenes. Non-limiting examples of terpenes include: Alpha Pinene, Limonene, Beta Pinene, Alpha Phellandrene, Terpinolene, Nerolidol, Nerol, Myrcene, Beta Caryophyllene, 7,8-dihydro-alpha-ionone, 7,8-dihydro-beta-ionone, Acetanisole, Acetic Acid, Acetyl Cedrene, Anethole, Anisole, Benzaldehyde, Bergamotene (Alpha-cis-Bergamotene) (Alpha-trans-Bergamotene), Bisabolol (Beta-Bisabolol), Alpha Bisabolol, Borneol, Bornyl Acetate, Butanoic/Butyric Acid, Cadinene (Alpha-Cadinene) (Gamma-Cadinene), Cafestol, Caffeic acid, Camphene, Camphor, Capsaicin, Carene (Delta-3-Carene), Carotene, Carvacrol, Dextro-Carvone, Laevo-Carvone, Alpha-Caryophyllene, Beta-Caryophyllene, Caryophyllene oxide, Cedrene (Alpha-Cedrene) (Beta-Cedrene), Cedrene Epoxide (Alpha-Cedrene Epoxide), Cedrol, Cembrene, Chlorogenic Acid, Cinnamaldehyde, Alpha-amyl-Cinnamaldehyde, Alpha-hexyl-Cinnamaldehyde, Cinnamic Acid, Cinnamyl Alcohol, Citronellal, Citronellol, Cryptone, Curcumene (Alpha-Curcumene) (Gamma-Curcumene), Decanal, Dehydrovomifoliol, Diallyl Disulfide, Dihydroactinidiolide, Dimethyl Disulfide, Eicosane/Icosane, Elemene (Beta-Elemene), Estragole, Ethyl acetate, Ethyl Cinnamate, Ethyl maltol, Eucalyptol/1,8-Cineole, Eudesmol (Alpha-Eudesmol) (Beta-Eudesmol) (Gamma-Eudesmol), Eugenol, Euphol, Farnesene, Farnesol, Fenchol (Beta-Fenchol), Fenchone, Geraniol, Geranyl acetate, Germacrenes, Germacrene B, Guaia-1 (10), 11-diene, Guaiacol, Guaiene (Alpha-Guaiene), Gurjunene (Alpha-Gurjunene), Herniarin, Hexanaldehyde, Hexanoic Acid, Humulene (Alpha-Humulene) (Beta-Humulene), Ionol (3-oxo-alpha-ionol) (Beta-Ionol), Ionone (Alpha-Ionone) (Beta-Ionone), Ipsdienol, Isoamyl Acetate, Isoamyl Alcohol, Isoamyl Formate, Isoborneol, Isomyrcenol, Isopulegol, Isovaleric Acid, Isoprene, Kahweol, Lavandulol, Limonene, Gamma-Linolenic Acid, Linalool, Longifolene, Alpha-Longipinene, Lycopene, Menthol, Methyl butyrate, 3-Mercapto-2-Methylpentanal, Mercaptan/Thiols, Beta-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (Beta-Myrcene), Gamma-Muurolene, Nepetalactone, Nerol, Nerolidol, Neryl acetate, Nonanaldehyde, Nonanoic Acid, Ocimene, Octanal, Octanoic Acid, P-Cymene, Pentyl butyrate, Phellandrene, Phenylacetaldehyde, Phenylethanethiol, Phenylacetic Acid, Phytol, Pinene, Beta-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, Alpha-Selinene, Alpha-Sinensal, Beta-Sinensal, Beta-Sitosterol, Squalene, Taxadiene, Terpin hydrate, Terpineol, Terpine-4-ol, Alpha-Terpinene, Gamma-Terpinene, Terpinolene, Thiophenol, Thujone, Thymol, Alpha-Tocopherol, Tonka Undecanone, Undecanal, Valeraldehyde/Pentanal, Verdoxan, Alpha-Ylangene, Umbelliferone, and Vanillin.

In some embodiments, the Cannabinoid Products described herein comprise a derivative of a terpene. Derivatives of terpenes include terpenoids, hemiterpenoids, monoterpenoids, sesquiterpenoids, sesterterpenoid, sesquarterpenoids, tetraterpenoids, triterpenoids, tetraterpenoids, polyterpenoids, isoprenoids, and steroids. Terpenoids, a.k.a. isoprenoids, are a large and diverse class of naturally occurring organic chemicals similar to terpenes, derived from five-carbon isoprene units assembled and modified in thousands of ways. Non-limiting examples of terpenoids include, Hemiterpenoids, 1 isoprene unit (5 carbons); Monoterpenoids, 2 isoprene units (10C); Sesquiterpenoids, 3 isoprene units (15C); Diterpenoids, 4 isoprene units (20C) (e.g. ginkgolides); Sesterterpenoids, 5 isoprene units (25C); Triterpenoids, 6 isoprene units (30C) (e.g. sterols); Tetraterpenoids, 8 isoprene units (40C) (e.g. carotenoids); and Polyterpenoid with a larger number of isoprene units.

### III. Cannabinoid Products

The Cannabinoid Products of the present disclosure provide a number of advantages over traditional oral cannabinoid formulations. For example, traditional cannabinoid formulations made with silicon dioxide are often unstable, requiring high quantities of other ingredients to stay solid. These traditional formulations are made with solvents such as ethanol in order to achieve loading of the cannabinoid into the silicon dioxide particles. These traditional formulations thus suffer from low cannabinoid drug to silicon dioxide particle ratios, creating low dose strength products. Traditional oral formulations also suffer from low bioavailability or exhibit uneven dissolution rates.

In contrast, embodiments of the Cannabinoid Products of the current disclosure exhibit linear dissolution rates in both fasted and fed media, thus providing sustained drug release for patients, and in some embodiments, alleviating the need for a patient to alter dietary regimens to accommodate taking a medication. In some embodiments, the Cannabinoid Products of the current disclosure do not require a diluent for the cannabinoid. Overcoming the need for a diluent can increase overall cannabinoid loading (e.g., in the form of higher cannabinoid to silicon dioxide ratios), or stronger cannabinoid dosages per weight volume of Cannabinoid product. Additionally, in some emboments, the lack of an ethanol diluent is preferablefor treatment of patients with low alcohol tolerance (e.g., recovering addicts or minors). In some embodiments, The inventorshave additionally discovered that, in some embodiments, heated non-crystalline cannabinoid oil has improved loading when compared to non-heated oils. In some embodiments, the heating further occurs before adding any ingredient other than silicon dioxcide particles, to ensure the oil is substantially loaded.

This disclosure provides Cannabinoid Products and oral pharmaceutical compositions (e.g., tablets and capsules) comprising the same. The Cannabinoid Products described herein comprise colloidal silicon dioxide particles, which contain pores, and non-crystalline cannabinoid oils. As a result, the non-crystalline cannabinoid oil does not separate from the powder ingredients of the Cannabinoid Product. Thus, the Cannabinoid Products described herein are homogenous free-flowing powders. Inventors have developed novel formulations for Cannabinoid Products and methods of making the same that provide for compressible tablets. Additional aspects of the Cannabinoid Products are described in more detail in the sections below.

In some embodiments, the Cannabinoid Products comprise cyclodextrin In some embodiments, the Cannabinoid Products do not comprise cyclodextrin. In some embodiments, the Cannabinoid Products do not require cyclodextrin. Cyclodextrin compounds include, α-cyclodextrin, β-cyclodextrin, 2-hydroxypropyl-β-cyclodextrin, sulfobutylether β-cyclodextrin sodium salt, randomly methylated β-cyclodextrin, branched β-cyclodextrin, γ-Cyclodextrin and derivatives thereof.

In some embodiments, the Cannabinoid Products comprise an emulsifier. In some embodiments, the Cannabinoid Products do not comprise an emulsifier. In some embodiments, Cannabinoid products do not require an emulsifier. Emulsifiers include PEG-7 Glyceryl Cocoate, PEG-20 Almond Glycerides, PEG 40 Sorbitane Hexaoleate, PEG 40 Sorbitane Perisostearate, PEG 10 Olive Glycerides, PEG-8 caprylic/capric glycerides (Labrafac CM 10-Gattefosse), Polyoxyethylene oleyl ether (EMULGEN 408-EMULGEN 430) respectively HLB=10-16.2, PEG Sorbilate Hexa oleate, Polysorbate 65 PE (20) sorbitan tristearate, Polyoxyethylene lauryl ether (G-3705), Polyoxyethylene lauryl ether (EMULGEN 106-EMULGEN 108-EMULGEN 109P-EMULGEN 120-EMULGEN 123P-EMULGEN 147-EMULGEN 150), PEG 25 Hydrogenated Castor Oil, Polyoxyethylene monostearate (Myrj 45), PEG 7 Glyceryl Cocoate (Sympatens-GMC/070), Glyceryl Stearate (and) PEG-100 Stearate Polysorbate 85, PEG-7 Olivate, PEG-20 sorbitan trioleate (Tween-85 Atlas/ICI), PEG-20 sorbitan tristearate (Tween 65 Atlas/ICI), PEG-25 hydrogenated castor oil (Simulsol 1292 Seppic), PEG-25 hydrogenated castor oil (Cerex ELS 250 Auschem SpA), PEG-25 trioleate (Tagat TO Goldschmidt), Polysorbate 85, PEG 8 Stearate, PEG 400 Monoleate, PEG Sorbitan Tetraoleate, PEG 400 Monoleate Polyoxyethylene monooleate, PEG-8 Oleate, PEG 400 Monostearate, PEG 400 Monostearate Polyoxyethylene monostearate Polyoxy-Ethylene Sucrose diester (Dierucat, PEG 35 Almond Glycerides, PEG 15 Glyceryl Isostearate, Polyoxyethylene alkyl phenol (Igepal Ca-630), PEG-35 castor oil (Cremophor EL/Cremophor EL-P BASF), Methyl-oxirane polymer with oxirane (Pluronic L-64 BASF), Polyoxyethylene alkyl ether (EMULGEN 707-EMULGEN MS-110-EMULGEN 709-EMULGEN LS-110-EMULGEN 1108-EMULGEN LS-114-EMULGEN 1118S-70-EMULGEN 1135S-70-EMULGEN 1150S-60), Polyglyceryl-3 Methyglucose Distearate=12 Oleth-10 Oleth-10/Polyoxyl 10 Oleyl Ether NF/(PEG 10 Oleyl Ether), PEG 8 Isooctylphenyl Ether, PEG 10 Stearyl Ether, PEG 35 Castor Oil, Polyethylene glycol 400 monolaurate, Polyoxyethylene distyrenated phenyl ether (EMULGEN A-60-EMULGEN A-90-EMULGEN A-500), PEG 10 Cetyl Ether, PEG 40 Castor Oil, PEG-8 Laurate, Acconon C-50 (PEG-32 Hydrogenated Palm Glycerides/EP/NF Stearoyl Macrogolglycerides (EP)/Stearoyl Polyoxylglycerides (NF)/Stearoyl polyoxyl-32 glycerides, PEG-35 hydrogenated castor oil (Cremophor RH40 BASF), PEG-40 hydrogenated castor oil (Cremophor RH40 BASF), PEG-1000 succinate (tocophersolan, D-α-tocopheryl/TPGS—Eastman), Polyoxyl-40-hydrogenated castor oil (Cremophor RH 40 BASF), Polyoxyethylene hydrogenated castor oil 40 (HCO-40 Nikkol), PEG 400 Monoluarate (Polyoxyethylene monolaurate), Polyoxyethylene sorbitan mono-oleate (Tween 80), Polyoxyethylene derivatives (EMULGEN B-66), PEG 10 Isooctylphenyl Ether, Polyoxyethylene cetyl ether (EMULGEN 220), Polysorbate 60 PE (20) sorbitan monostearate, PEG 12 Tridecyl Ether, PEG 18 Tridecyl Ether, PEG 40 Hydrogenated Castor Oil, Acconon C-44 (polyoxyethylene 32 lauric glycerides/PEG-32 Lauric Glycerides/Lauroyl Macrogolglycerides (EP)/Lauroyl Polyoxyglycerides (NF)/Lauroyl Polyoxyl-32 glycerides, PEG-60 hydrogenated castor oil (HCO-60-Nikko), PEG-8 caprylic/capric glycerides (Labrasol-Gattefosse), Polysorbate 60 NF, Poloxyethylene sorbitan monostearate, Polysorbate 60, PEG-60 Almond Glycerides, PEG 20 Glyceryl Stearate, PEG 20 Stearate, PEG-20 Methyl Glucose Sesquistearate, Polysorbate 80, PEG-20 sorbitan monooleate (Tween-80 Atlas/ICI), Polyoxyethylene sorbitan monooleate, Polisorbate 60 (PS 60), Polyoxyethylene sorbitan monolaurate (Tween 20), Polysorbate 80, PEG 20 Stearyl Ether, PEG 20 Oleyl Ether, Polysorbate 80 PE (20) sorbitan monooleate, PEG 20 Cetyl Ether, PEG (20) Hexadecyl Ether, PEG 60 Hydrogenated Castor Oil, PEG 30 Stearate, PEG 75 Lanolin, Polysorbate 20, Polysorbate 20 NF, Polyoxyethylene lauryl ether (Brij 35), Polysorbate 20, Eumulgin® L (PPG-1-PEG-9 Lauryl Glycol Ether/Glycols, 1,2-, C12-16, ethoxylated propoxylated), PEG 23 Lauryl Ether, PEG-20 sorbitan monolaurate (Tween20 Atlas/ICI), Polyoxy-Ethylene Sucrose diester Dimyristate, PEG 40 Stearate, Polyoxy-Ethylene Sucrose diester Dinnyristate, Polyoxy-Ethylene Sucrose diester Dipalmitate, PEG 50 Stearate, PEG 40 Isooctylphenyl Ether, Polyoxy-Ethylene Sucrose diester Dioleate, Polyoxyethylene-polyoxypropylene copolymers (Pluronic F 127—BASF), PEG 100 Stearate, Polyoxyethylene myristyl ether (EMULGEN 4085), PEG-80 Sorbitan Laurate Linoleamide DEA, Stearamide MEA, Cetearyl Glucoside, Triethanolamine oleate, Sucrose monostearate, Oleth-10/Polyoxyl 10 Oleyl Ether NF, Steareth-10, Ceteth-10, Cocamide MEA, Isosteareth-20, Sucrose laurate, Sucrose stearate, Lauramide DEA, Stearic Acid, Ceteareth-20, Oleth-20, Steareth-20, Steareth-21, Cetearyl Alcohol, Ceteth-20, Isoceteth-20, Ceteth-20, Sucrose palmitate, Laureth-23, Sodium oleate 16.9, Potassium oleate, Steareth-100, Sodium stearoyl-2-lactylate, Sodium stearoyl lactylate and combinations thereof.

### III-A. Cannabinoid Oils

In embodiments, the Cannabinoid Products comprise a non-crystalline cannabinoid oil. In some embodiments, a cannabinoid oil refers to a composition produced by extracting cannabinoids from a Cannabis plant or Cannabis plant part(s). In some embodiments, the cannabinoid oil is chemically or biosynthetically produced. In some embodiments, the cannabinoid oil is non-crystalline cannabinoid oil.

The present disclosure teaches formulations produced with non-crystalline cannabinoid oils. This is distinct from prior art formulations produced with powdered cannabinoid isolates that are solids (and therefore easily made into oral tablets or capsules), but which suffer from low bioavailability. The Cannabinoid Products of the present disclosure are made with liquid, sometimes viscous, cannabinoid oil, which is made into a solid (e.g., powder form) due to the liquid being loaded onto cavities contained within the colloidal silicon dioxide particles. In some embodiments, the cannabinoid oil is heated prior to being mixed with the colloidal silica particles. Without wishing to be bound by any one theory, the inventors believe that the heating step improves loading by reducing viscosity of the cannabinoid oil, and further dissolves any crystals that may have formed in the cannabinoid oil during storage.

In embodiments, a cannabinoid oil is produced using any suitable extraction method. In embodiments, the extraction method is selected from the group consisting of maceration, percolation, solvent extraction, steam distillation, and vaporization. General protocols for the preparation of cannabinoid oils are described in the following patent documents: U.S. Pat. Nos. 8,603,515; 9,730,911; 7,700,368, 10,159,908, U.S.

Pub. No. 2019/0151771, U.S. Pub. No. 2018/0078874, U.S. Pub. No. 2020/0080021, U.S. Pub. No. 2020/0048214, U.S. Pub. No. 2020/0048215, and U.S. patent Ser. No. 10/555,914, which are each incorporated by reference herein in their entireties.

Solvent extraction may be carried out using essentially any solvent that dissolves cannabinoids/cannabinoid acids, such as for example C1 to C5 alcohols (e.g. ethanol, methanol), C4-C12 alkanes (e.g. hexane or butane), Norflurane (HFA134a), HFA227, and carbon dioxide. When solvents such as those listed above are used, the resultant primary extract typically contains non-specific lipid-soluble material or “ballast” e.g. waxes, wax esters and glycerides, unsaturated fatty acid residues, terpenes, carotenes, and flavonoids. The primary extract may be further purified for example by “winterization”, which involves chilling to −20° C. followed by filtration to remove waxy ballast, supercritical or subcritical extraction, vaporization, distillation, and chromatography.

In some embodiments, the cannabinoid oil may be obtained by carbon dioxide (CO2) extraction followed by a secondary extraction, e.g. an ethanolic precipitation, to remove a substantial proportion of non-cannabinoid materials. In some embodiments, a cannabinoid oil is produced by a process comprising extraction with liquid CO₂ under sub-critical or super-critical conditions, and then a further extraction (e.g., an ethanolic precipitation) to remove significant amounts of ballast. If it is intended to prepare free cannabinoids from the Cannabis plant parts, then the plant parts preferably heated to a defined temperature for a defined period of time in order to decarboxylate cannabinoid acids to free cannabinoids prior to extraction of the botanical drug substance.

In some embodiments, a cannabinoid oil is prepared according to a process comprising the following steps: i) optional decarboxylation of the plant material, ii) extraction with liquid CO₂ (in some embodiments under sub-critical conditions), to produce a crude botanical drug substance, iii) precipitation with C₁-C₅ alcohol to reduce the proportion of non-target materials, iv) removal of the precipitate (preferably by filtration), v) optional treatment with activated charcoal, and vi) evaporation to remove C₁-C₅ alcohol and water, thereby producing a final botanical drug substance.

In some embodiments, a cannabinoid oil is prepared from plant parts according to a process comprising the following steps: i) CO₂ extraction for plant terpenes, ii) ethanol extraction for crude cannabinoids, plant waxes, and plant oils (crude extract); iii) winterization of the crude extract at −80° C. for 24 hours; and iv) complete ethanol recovery and in-vessel decarboxylation of winterized crude before fractional distillation of cannabinoids. In some embodiments, the cannabinoid oil is heated to ensure that it is non-crystalline cannabinoid oil.

In some embodiments, a cannabinoid oil described herein is extracted from Cannabis using any method known in the art and subsequently winterized.

Cannabis oil used in the Cannabinoid Products of the present disclosure does not need to be diluted in any solvents. In some embodiments, the lack of solvents permits for more potentent non-crystalline cannabinoid oils to be loaded onto the colloidal silica particles, thereby increasing the overall dosage of the Cannabinoid Product.

In some embodiments, the non-crystalline cannabinoid oil used in the Cannabinoid Products contains about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% cannabinoid content by weight of the cannabinoid oil, including all subranges, ranges, and values therebetween. Thus, in some embodiments, the cannabinoid oil contains between 1-100% cannabinoids by weight.

In some embodiments, the cannabinoid oil used in the Cannabinoid Products contains at least about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% cannabinoid content by weight (i.e. cannabinoid weight over cannabinoid oil weight). That is, in some embodiments, the non-crystalline cannabinoid oil of the present disclosure can be up to 100% by weight of a cannabinoid selected from the group consisting of: CBD, THC, D8 THC or A8 THC, THCV, CBDA, CBDV, THCA, CBG, CBGA, CBN, CBNA, CBDVA, CBCA, and CBC.

In embodiments, the non-crystalline cannabinoid oil comprises from 80% to 99% w/w cannabinoid content, for example 80%-99% w/w cannabidiol (CBD) and less than 0.1% delta-9-tetrahydrocannabinol by weight. In embodiments, the non-crystalline cannabinoid oil comprises from 80% to 99% cannabidiol (CBD) and less than 0.3% delta-9-tetrahydrocannabinol by weight. In embodiments, the oil comprises cannabigerol (CBG), cannabinol (CBN), or a combination thereof.

In embodiments, the non-crystalline cannabinoid oil comprises up to 50% CBD and up to 30% other cannabinoids by weight. In embodiments, the non-crystalline cannabinoid oil does not form crystals. In embodiments, the non-crystalline cannabinoid oil comprises CBD, CBG, CBN, and cannabichromene (CBC).

In embodiments, the non-crystalline cannabinoid oil comprises 70-80% CBN and 8-15% other cannabinoids by weight (i.e., CBC, CBG, CBD, cannabidivarin (CBDV), Cannabicitran (CBT), and combinations thereof).

In embodiments, the non-crystalline cannabinoid oil comprises 70-85% CBG and 8-15% other cannabinoids (i.e., CBC, CBD, CBN, CBDV, CBT, and combinations thereof) by weight. In embodiments, the oil contains about 25% CBD and up to 15% CBC and CBN by weight.

In embodiments, the non-crystalline cannabinoid oil comprises from 80% to 92% delta-8-tetrahydrocannabinol. In embodiments, the non-crystalline cannabinoid oil comprises about 84.8% delta-8-tetrahydrocannabinol by weight. In embodiments, the non-crystalline cannabinoid oil comprises about 90.5% delta-8-tetrahydrocannabinol by weight.

In embodiments, the non-crystalline cannabinoid oil comprises about 73.2% THC by weight.

The cannabinoid dosage strength of Cannabinoid Products is a consequence of the cannabinoid content of the non-crystalline cannabinoid oil and how much of that oil can be loaded into silicon dioxide particles. As noted earlier, the non-crystalline oil of the present disclosure does not require dilution in ethanol or other solvents, and is therefore capable of comprising higher cannabinoid contents than traditional products. In some embodiments, Cannabinoid Products of the present disclosure are further capable of loading higher amounts (e.g., wt/wt) of non-crystalline cannabinoid oil into the silicon dioxide particles.

In embodiments, the Cannabinoid Products comprise from about 1% to about 85% non-crystalline cannabinoid oil by weight. In embodiments, the Cannabinoid Products comprise from about 1% to about 50% non-crystalline cannabinoid oil by weight. In embodiments, the Cannabinoid Products comprise from about 1% to about 30% non-crystalline cannabinoid oil by weight. In embodiments, the Cannabinoid Products comprise from about 10% to about 60% non-crystalline cannabinoid oil by weight. For example, in embodiments, the Cannabinoid Products comprise about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60% about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, or about 85%, non-crystalline cannabinoid oil by weight of the composition, including all subranges, ranges, and values therebetween.

In some embodiments, the cannabinoid content of the Cannabinoid Products can be defined directly in terms of the amount of loaded non-crystalline cannabinoid (e.g. amount of THC, CBD, CBN, CBG, etc.). Thus, in some embodiments the present disclosure teaches Cannabinoid products comprising about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, or about 88% cannabinoid content by weight of the composition, including all ranges and subranges therebetween.

### III-B. Colloidal Silicon Dioxide Particles

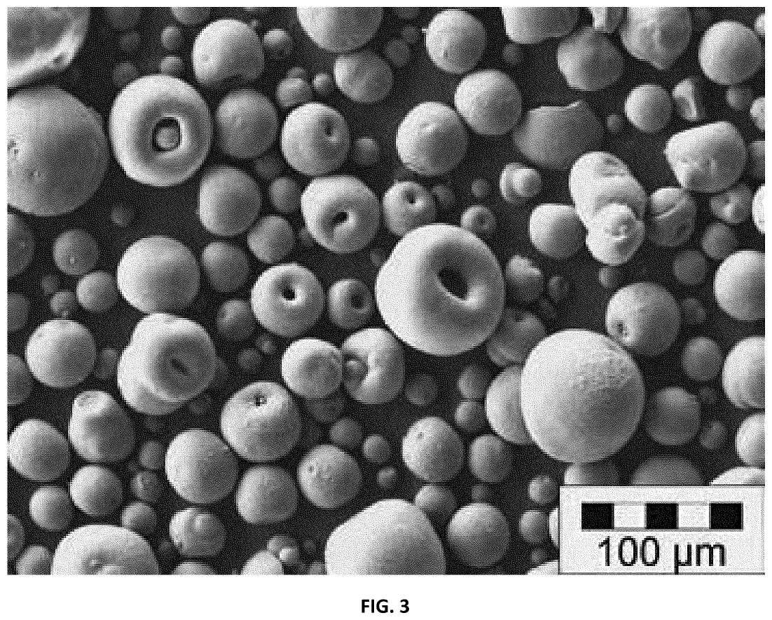

In embodiments, the Cannabinoid Products described herein comprise colloidal silicon dioxide particles. The term “colloidal silicon dioxide” may refer to a silica product produced by flame hydrolysis. Colloidal silicon dioxide particles are comprised of amorphous synthetic silicon dioxide with pores. FIG. 3 shows an image of colloidal silicon dioxide particles. The following document describes colloidal silicon dioxide particles and is incorporated by reference herein in its entirety: EVONIK®; Technical Information 1414: AEROPERL® 300 Pharma Improving the dissolution of poorly soluble APIs.

In some embodiments, colloidal silica (e.g., colloidal silicon dioxide) of the present disclosures have a low bulk density and high surface area. In some embodiments, the colloidal silica particles have a mean particle diameter of 10 to 250 micron (e.g., as determined according to the laser diffraction method). In some embodiments, the colloidal silica particles have a BET surface area of 40 to 400 m²/g (e.g., as determined according to DIN 66 131 with nitrogen). In some embodiments, the colloidal silica particles have a pore volume of about 0.5 to 2.5 mL/g, wherein less than about 5% of the overall pore volume has a pore diameter of less than about 5 nm, the remainder being mesopores and macropores. In some embodiments, the particles of the colloidal silica have a mean grain (particle) diameter of 10-120 micron.

In embodiments, the colloidal silicon dioxide comprises Aerosil 200 Pharma, Aerosil 300 Pharma, Aerosil VV Pharma, Aerosil R 972 Pharma, and/or Aeroperl 300 Pharma.

In embodiments, the size of colloidal silicon dioxide particles ranges from about 20 μm to about 60 μm, as determined by scanning electron microscopy. The particle size refers to the diameter of the particle. In embodiments, the average particle size of the colloidal silicon dioxide particles is about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 51 μm, about 52 μm, about 53 μm, about 54 μm, about 55 μm, about 56 μm, about 57 μm, about 58 μm, about 59 μm, about 60 μm, about 61 μm, about 62 μm, about 63 μm, about 64 μm, about 65 μm, about 66 μm, about 67 μm, about 68 μm, about 69 μm, about 70 μm, about 71 μm, about 72 μm, about 73 μm, about 74 μm, about 75 μm, about 76 μm, about 77 μm, about 78 μm, about 79 μm, about 80 μm, about 81 μm, about 82 μm, about 83 μm, about 84 μm, about 85 μm, about 86 μm, about 87 μm, about 88 μm, about 89 μm, about 90 μm, about 91 μm, about 92 μm, about 93 μm, about 94 μm, about 95 μm, about 96 μm, about 97 μm, about 98 μm, about 99 μm, about 100 μm, about 101 μm, about 102 μm, about 103 μm, about 104 μm, about 105 μm, about 106 μm, about 107 μm, about 108 μm, about 109 μm, about 110 μm, about 111 μm, about 112 μm, about 113 μm, about 114 μm, about 115 μm, about 116 μm, about 117 μm, about 118 μm, about 119 μm, or about 120 μm including all values, subranges, and ranges therebetween.

In embodiments, the colloidal silicon dioxide particles have pores. In embodiments, the pores have a volume from 0.1 milliliters (mL) per gram (g) to about 5 mL/g. In embodiments, the pores have a volume from about 1.5 mL/g to about 1.9 mL/g. In embodiments, the pores have a volume of about 0.1 mL/g, about 0.2 mL/g, about 0.3 mL/g, about 0.4 mL/g, about 0.5 mL/g, about 0.6 mL/g, about 0.7 mL/g, about 0.8 mL/g, about 0.9 mL/g, about 1 mL/g, about 1.1 mL/g, about 1.2 mL/g, about 1.3 mL/g, about 1.4 mL/g, about 1.5 mL/g, about 1.6 mL/g, about 1.7 mL/g, about 1.8 mL/g, about 1.9 mL/g, about 2 mL/g, about 2.1 mL/g, about 2.2 mL/g, about 2.3 mL/g, about 2.4 mL/g, about 2.5 mL/g, about 2.6 mL/g, about 2.7 mL/g, about 2.8 mL/g, about 2.9 mL/g, about 3 mL/g, about 3.1 mL/g, about 3.2 mL/g, about 3.3 mL/g, about 3.4 mL/g, about 3.5 mL/g, about 3.6 mL/g, about 3.7 mL/g, about 3.8 mL/g, about 3.9 mL/g, about 4 mL/g, about 4.1 mL/g, about 4.2 mL/g, about 4.3 mL/g, about 4.4 mL/g, about 4.5 mL/g, about 4.6 mL/g, about 4.7 mL/g, about 4.8 mL/g, about 4.9 mL/g, or about 5 mL/g, including any values, subranges, and ranges therebetween. In embodiments, the pores of the colloidal silicon dioxide particles have volumes ranging from about 1.5 milliliters (mL)/gram (g) to about 1.9 mL/g. For example, the volume of the particle may be about 1.5 mL/g, about 1.55 mL/g, about 1.6 mL/g, about 1.65 mL/g, about 1.7 mL/g, about 1.75 mL/g, about 1.8 mL/g, about 1.85 mL/g, or about 1.9 mL/g.

In embodiments, the Cannabinoid Products comprise about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90% colloidal silicon dioxide particles by weight of the composition, including all ranges and subranges therebetween. In embodiments, the Cannabinoid Products comprise from about 20% to about 90%, from about 25% to about 50%, from about 25% to about 35%, from about 35% to about 45%, or about 29.1% to about 58.2% colloidal silicon dioxide particles by weight. In embodiments, the Cannabinoid Products comprise from about 29.1% to about 58.2% colloidal silicon dioxide particles by weight.

### III-C. Filler

In embodiments, the Cannabinoid Products described herein comprise a filler. Non-limiting examples of fillers include silicon dioxide (not loaded with any cannabinoid), titanium dioxide, alumina, talc, kaolin, powdered cellulose, microcrystalline cellulose, urea, sodium chloride, as well as saccharides, or combinations thereof. Any suitable saccharide may be used in a Cannabinoid Product described herein. As used herein, the term “saccharide” includes sugar alcohols, monosaccharides, disaccharides, and oligosaccharides. Exemplary sugar alcohols include, but not limited to, xylitol, mannitol, sorbitol, erythritol, lactitol, pentitol, and hexitol. Exemplary monosaccharides include, but are not limited to, glucose, fructose, aldose and ketose. Exemplary disaccharides include, but are not limited to, sucrose, isomalt, lactose, trehalose, and maltose. Exemplary oligosaccharides include, but are not limited to, fructo-oligosaccharides, inulin, galacto-ologosaccharides, and mannan-oligosaccharides. In some embodiments, the saccharide is sorbitol, mannitol, or xylitol. In some embodiments, the saccharide is sorbitol. In some embodiments, the saccharide is sucrose.

In embodiments, the filler is mannitol. The chemical structure of mannitol is below:

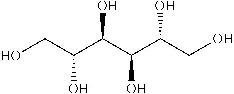 [SMILES: OC[C@@H](O)[C@@H](O)[C@H](O)[C@H](O)CO]

In embodiments, the filler is microcrystalline cellulose. In embodiments, the filler is silicified microcrystalline cellulose. Silicified microcrystalline cellulose is a combination of microcrystalline cellulose and colloidal silicon dioxide in which the colloidal silicon dioxide binds to the microcrystalline cellulose by noncovalent interactions. In embodiments, silicified microcrystalline cellulose has an average particle size as measured by laser diffraction of from about 40 μm to about 150 μm. For example, in embodiments, the average particle size of the silicified microcrystalline cellulose is about 40 μm, about 41 μm, about 42 μm, about 43 μm, about 44 μm, about 45 μm, about 46 μm, about 47 μm, about 48 μm, about 49 μm, about 50 μm, about 51 μm, about 52 μm, about 53 μm, about 54 μm, about 55 μm, about 56 μm, about 57 μm, about 58 μm, about 59 μm, about 60 μm, about 61 μm, about 62 μm, about 63 μm, about 64 μm, about 65 μm, about 66 μm, about 67 μm, about 68 μm, about 69 μm, about 70 μm, about 71 μm, about 72 μm, about 73 μm, about 74 μm, about 75 μm, about 76 μm, about 77 μm, about 78 μm, about 79 μm, about 80 μm, about 81 μm, about 82 μm, about 83 μm, about 84 μm, about 85 μm, about 86 μm, about 87 μm, about 88 μm, about 89 μm, about 90 μm, about 91 μm, about 92 μm, about 93 μm, about 94 μm, about 95 μm, about 96 μm, about 97 μm, about 98 μm, about 99 μm, about 100 μm, about 101 μm, about 102 μm, about 103 μm, about 104 μm, about 105 μm, about 106 μm, about 107 μm, about 108 μm, about 109 μm, about 110 μm, about 111 μm, about 112 μm, about 113 μm, about 114 μm, about 115 μm, about 116 μm, about 117 μm, about 118 μm, about 119 μm, about 120 μm, about 121 μm, about 122 μm, about 123 μm, about 124 μm, about 125 μm, about 126 μm, about 127 μm, about 128 μm, about 129 μm, about 130 μm, about 131 μm, about 132 μm, about 133 μm, about 134 μm, about 135 μm, about 136 μm, about 137 μm, about 138 μm, about 139 μm, about 140 μm, about 141 μm, about 142 μm, about 143 μm, about 144 μm, about 145 μm, about 146 μm, about 147 μm, about 148 μm, about 149 μm, or about 150 μm, including all subranges, ranges, and values therebetween. In embodiments, the silicified microcrystalline cellulose has an average particle size as measured by laser diffraction of about 65 μm.

In embodiments, the Cannabinoid Product comprises from about 1% to about 60% filler by weight. In embodiments, the Cannabinoid Product comprises from about 20% to about 45% filler by weight. In embodiments, the Cannabinoid Products comprise from about 30% to about 60% filler by weight. In embodiments, the Cannabinoid Products comprise from about 5% to about 60% filler by weight. In embodiments, the Cannabinoid Products comprise from about 12.3% to about 43.4% filler by weight. For example, in embodiments, the Cannabinoid Product comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% filler by weight of the composition, including any values, subranges, and ranges therebetween. In embodiments, the Cannabinoid Product comprises about 25% filler by weight.

### III-D. Disintegrant

In embodiments, the Cannabinoid Products described herein comprise a disintegrant. Disintegrants are used to facilitate disintegration of the tablet. Non-limiting examples of disintegrants include starches, clays, celluloses, algins, gums, crosslinked polymers, lightly crosslinked polyvinyl pyrrolidone, corn starch, potato starch, maize starch and modified starches, croscarmellose sodium, crospovidone, sodium starch glycolate, and combinations and mixtures thereof. In embodiments, the disintegrant is sodium starch glycolate or croscarmellose sodium.

In embodiments, the Cannabinoid Product comprises from about 0.5% to about 10% disintegrant by weight. In embodiments, the Cannabinoid Product comprises from about 0.5% to about 5% disintegrant by weight. In embodiments, the Cannabinoid Product comprises from about 2% to about 5.5% disintegrant by weight. In embodiments, the Cannabinoid Product comprises from about 2.5% to about 7.5% disintegrant by weight. For example, in embodiments, the Cannabinoid Product comprises about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10% disintegrant by weight of the composition, including any values, subranges, and ranges therebetween. In embodiments, the disintegrant is sodium starch glyolate or croscarmellose sodium.

### III-E. Lubricant

In embodiments, the Cannabinoid Products described herein comprise a lubricant. Lubricants are used to facilitate tablet manufacture, promoting powder flow and preventing particle capping (i.e., particle breakage) when pressure is relieved. Non-limiting examples of lubricants include: magnesium stearate, calcium stearate, stearic acid, glyceryl behenate, talc, mineral oil (in PEG), hydrogenated vegetable oil (e.g., comprised of hydrogenated and refined triglycerides of stearic and palmitic acids), and sodium stearyl fumarate. In embodiments, the lubricant is sodium stearyl fumarate.

In embodiments, the Cannabinoid Product comprises from about 0.5% to about 10% lubricant by weight. In embodiments, the Cannabinoid Product comprises from about 0.1% to about 1% lubricant by weight. In embodiments, the Cannabinoid Product comprises from about 1% to about 2.7% lubricant by weight. For example, in embodiments, the Cannabinoid Product comprises about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, or about 10% lubricant by weight of the composition, including all ranges and subranges therebetween.

### III-F. Cannabinoid Isolates

In embodiments, the Cannabinoid Products described herein comprise a cannabinoid isolate. In embodiments, the Cannabinoid Product contains a cannabinoid isolate containing any cannabinoid described herein. In embodiments, the Cannabinoid Product comprises a cannabinoid isolate comprising CBD. In embodiments, the Cannabinoid Product comprises a cannabinoid isolate comprising CBG. In embodiments, the Cannabinoid Product comprises 1, 2, 3, 4, 5, 6, 7, 8, 9, or 10 cannabinoid isolates (i.e. isolates of different cannabinoids).

In embodiments, the Cannabinoid Products comprise from about 1% to about 70% cannabinoid isolate by weight. For example, in embodiments, the Cannabinoid Products comprise about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, or about 70% cannabinoid isolate by weight of the composition, including all ranges and subranges therebetween. In some embodiments, the cannabinoid isolate is entirely melted, and therefore non-crystalline.

### III-G. Flavorings

In embodiments, the Cannabinoid Products described herein comprise a flavoring. Non-limiting examples of flavorings include vanilla, citrus, lemon, orange, lime, grapefruit, yazu, sudachi, apple, pear, peach, grape, blueberry, strawberry, raspberry, cherry, plum, pineapple, watermelon, apricot, banana, melon, apricot, ume, cherry, raspberry, blackberry, tropical fruit, mango, mangosteen, pomegranate, papaya, spearmint, cinnamon, wintergreen, peppermint, eucalyptus, and anise.

In embodiments, the flavoring is a terpene. Non-limiting examples of terpenes are found in Section II-B of this disclosure. In embodiments, the terpene comprises myrcene, linalool, or a combination thereof.

In embodiments, the Cannabinoid Products comprise from about 0.01% and about 5% flavorings by weight. For example, the Cannabinoid Products may comprise about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, or about 5% flavorings by weight of the composition, including all ranges and subranges therebetween. In embodiments, the Cannabinoid Products comprise about 0.2% flavoring by weight. In embodiments, the Cannabinoid Products comprise about 0.4% flavoring by weight.

### III-H. Exemplary Cannabinoid Products

In embodiments, provided herein is a solid Cannabinoid Product comprising: (a) non-crystalline cannabinoid oil; and (b) colloidal silicon dioxide particles comprising internal storage cavities, wherein the non-crystalline cannabinoid oil is substantially loaded into the colloidal silicon dioxide storage particles. In some embodiments, the loaded non-crystalline cannabinoid oil to colloidal silicon dioxide particle ratio is about 0.5:1 to 3:1 by weight.

In embodiments, provided herein is a solid Cannabinoid Product comprising: (a) non-crystalline cannabinoid oil; and (b) colloidal silicon dioxide particles comprising internal storage cavities, wherein at least 90% of the non-crystalline cannabinoid oil is loaded into the colloidal silicon dioxide storage particles. In some embodiments, the non-crystalline cannabinoid oil content is at least 30% w/w of the colloidal silicon dioxide particle content.

In embodiments, provided herein is a cannabinoid daily patient dose comprising: (a) non-crystalline cannabinoid oil comprising a cannabinoid; and (b) colloidal silicon dioxide particles comprising internal storage cavities, wherein, the non-crystalline cannabinoid oil is loaded into the colloidal silicon dioxide particles comprising internal storage cavities. In some embodiments, said daily dose comprises greater than 50 mg of the cannabinoid.

In embodiments, provided herein is a solid Cannabinoid Product comprising: (a) non-crystalline cannabinoid oil; and (b) colloidal silicon dioxide particles, wherein the non-crystalline cannabinoid oil is substantially loaded into the colloidal silicon dioxide particles comprising storage particles. Optionally, the product exhibits a linear dissolution rate as measured in 1% Tween 80 media, Fasted State Simulated Intestinal Fluid (FaSSIF) media, and/or Fed State Simulated Intestinal Fluid (FeSSIF) media. In embodiments, the Cannabinoid Product for a tablet is selected from any formulation of Table 1.

TABLE 1
Exemplary Cannabinoid Products for Tablets

| | I | J | K | L | M | M[H] |
|---|---|---|---|---|---|---|
| | %(w/w) | %(w/w) | %(w/w) | %(w/w) | %(w/w) | %(w/w) |
| Cannabinoid oil | 41.9 | 22.1 | 22.1 | 22.1 | 22.1 | 19.4 |
| (non crystalline) | | | | | | |
| Colloidal silicon | 41.9 | 58.2 | 58.2 | 29.1 | 29.1 | 31.1 |
| dioxide particles | | | | | | |
| Silicified | | | | | | 46.6 |
| Microcrystalline | | | | | | |
| cellulose (filler) | | | | | | |
| Mannitol (filler) | 12.3 | 14.3 | 14.3 | 40.7 | 43.4 | |
| Sodium starch | 2.0 | 2.7 | 2.7 | 5.5 | 2.7 | |
| glycolate | | | | | | |
| (disintegrant) | | | | | | |
| Croscarmellose | | | | | | 1.9 |
| sodium | | | | | | |
| (disintegrant) | | | | | | |
| Sodium stearyl | 2.0 | 2.7 | 2.7 | 2.7 | 2.7 | 1.0 |
| fumarate | | | | | | |
| (lubricant) | | | | | | |

In embodiments, the Cannabinoid Product for a capsule is selected from any one of the formulations of Table 2.

TABLE 2
Exemplary Cannabinoid Products for Capsules

| | G[C] | | E[C] | D[C] |
|---|---|---|---|---|
| | %(w/w) | F[C] %(w/w) | %(w/w) | %(w/w) |
| Cannabidiol (CBD) | 40.4 | 40.4 | 40.4 | 21.7 |
| Isolate | | | | |
| Cannabigerol (CBG) | 11.7 | 11.7 | 11.7 | 8.5 |
| Isolate | | | | |
| Cannabinoid oil | 8.1 | 8.1 | 8.1 | 2.2 |
| Colloidal silicon | 39 | 39 | 39 | 32.4 |
| dioxide | | | | |
| Silicified | | | | 34.8 |
| Microcrystalline | | | | |
| cellulose (filler) | | | | |
| Sodium stearyl | 0.4 | 0.4 | 0.4 | 0.4 |
| fumarate (lubricant) | | | | |
| Myrcene (flavoring) | 0.2 | 0.2 | 0.2 | |
| Linalool (flavoring) | 0.2 | 0.2 | 0.2 | |

### Cannabinoid Product Properties

In embodiments, the Cannabinoid Products of the present disclosure exhibit desirable properties over traditional oral cannabinoid formulations. For example, the cannabinoid products exhibit linear dissolution rates in both fasted and fed media that allow for sustained drug release for patients. The Cannabinoid Products of the current disclosure do not require a diluent, in contrast with traditional products, which often require diluents, such as ethanol. Ethanol is often restricted in certain age groups, such as children, and may further interact with other medications a subject is taking. Removing the requirement of a diluent allows for an increased cannabinoid loading capacity in the presently disclosed cannabinoid products. An increased non-crystalline cannabinoid oil: colloidal silicon dioxide ratio within the cannabinoid product can generate products with higher cannabinoid doses within the same quantity of silicon dioxide particles. These higher doses can be desirable because, in some instances, the FDA limits to amount of silicon dioxide that may be consumed. The general propertie and benefits of the presently disclosed Cannabinoid Products are discussed in more detail, below.

### Dissolution Rates

Cannabinoid Products of the present disclosure exhibit improved dissolution and bioavailability compared to traditional formulations. In embodiments, the Cannabinoid Products of the present disclosure exhibit a linear dissolution rate throughout 5-60 minutes after contacting 1% Tween 80 media, Fasted State Simulated Intestinal Fluid (FaSSIF) media, and/or Fed State Simulated Intestinal Fluid (FeSSIF) media. These media, which are described in further detail in the Examples, serve as models for dissolution within patients.

In embodiments, the linear dissolution rate is defined by conformance with an R² value limit, said R² value calculated based on a linear regression of active ingredient (i.e. cannabinoid) dissolution within the media described herein. An R² value is a statistical measure that represents the proportion of the variance for a dependent variable that's explained by an independent variable in a regression model. Thus, R² on a linear model is indicative of the dissolution data's fit to a calculated linear model. High R² values are indicative that measured dissolution match the predicted linear model (See Nakagawa & Schielzeth, Methods Ecol Evol, 4:133-142 (2012), which is incorporated by reference in its entirety). R² is calculated using the following equation: R²=1−RSS/TSS, where RSS is the sum of squares of residuals and TSS is the total sum of squares.

For the purposes of this disclosure, Cannabinoid Products exhibit a linear dissolution rate, if the dissolution values measured in 1% Tween 80 media, Fasted State Simulated Intestinal Fluid (FaSSIF) media, and/or Fed State Simulated Intestinal Fluid (FeSSIF) media, exhibit an R² value of at least 0.85 against a linear (Y=mX+b) regression.

Thus, in embodiments, the Cannabinoid Products of the present disclosure exhibit an R² value of at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.99, at least about 0.99, or about 1 throughout 5-60 minutes after contacting 1% Tween 80 media, Fasted State Simulated Intestinal Fluid (FaSSIF) media, and/or Fed State Simulated Intestinal Fluid (FeSSIF) media, including all ranges and subranges therebetween.

### Ratio of Non-Crystalline Cannabinoid Oil to Colloidal Silicon Dioxide Particles

In some embodiments, the Cannabinoid Products of the present disclosure are capable of higher cannabinoid oil loading into colloidal silicon dioxide particles than traditional products. For example, In some embodiments, the higher loading is defined via a wt/wt ratio of non-crystalline cannabinoid oil to colloidal silicon dioxide particles in the composition, wherein the non-crystalline cannabinoid oil in the ratio refers to the amount of non-crystalline cannabinoid oil that is loaded into the colloidal silicon dioxide particles. In some embodiments, the Cannabinoid Products exhibit higher non-crystalline cannabinoid oil to colloidal silicon dioxide particles than were previously achievable.

In embodiments, the non-crystalline cannabinoid oil to colloidal silicon dioxide particles ratio is about 0.5:1, about 0.6:1, about 0.7:1, about 0.75:1, about 0.8:1, about 0.9:1, about 1:1, about 1.1:1, about 1.2:1, about 1.25:1, about 1.3:1, about 1.4:1, about 1.5:1, about 1.6:1, about 1.7:1, about 1.75:1, about 1.8:1, about 1.9:1, about 2:1, about 2.1:1, about 2.2:1, about 2.25:1, about 2.3:1, about 2.4:1, about 2.5:1, about 2.6:1, about 2.7:1, about 2.75:1, about 2.8:1, about 2.9:1, about 3:1, about 3.1:1, about 3.2:1, about 3.25:1, about 3.3:1, about 3.4:1, about 3.5:1, about 3.6:1, about 3.7:1, about 3.75:1, about 3.8:1, about 3.9:1, about 4:1, about 4.25:1, about 4.5:1, about 4.75:1, about 5:1, about 5.25:1, about 5.5:1, about 5.75:1, about 6:1, about 6.25:1, about 6.5:1, about 6.75:1, about 7:1, about 7.25:1, about 7.5:1, about 7.75:1, about 8:1, about 8.25:1, about 8.5:1, about 8.75:1, about 9:1, about 9.25:1, about 9.5:1, about 9.75:1, or about 10:1 by weight, including all values, subranges, and ranges therebetween.

In embodiments, the non-crystalline cannabinoid oil to colloidal silicon dioxide particles ratio is from about 0.5:1 to about 0.75:1, from about 1:1 to about 1.25:1, from about 1.25:1 to about 1.5:1, from about 1.5:1 to about 1.75:1, from about 1.75:1 to about 2:1, from about 2:1 to about 2.25:1, from about 2.25:1 to about 2.5:1, from about 2.5:1 to about 2.75:1, from about 2.75:1 to about 3:1, from about 3:1 to about 3.25:1, from about 3.25:1 to about 3.5:1, from about 3.5:1 to about 3.75:1, from about 3.75:1 to about 4:1, from about 4:1 to about 4.25:1, from about 4.25:1 to about 4.5:1, from about 4.5:1 to about 4.75:1, from about 4.75:1 to about 5:1, from about 5:1 to about 5.25:1, from about 5.25:1 to about 5.5:1, from about 5.5:1 to about 5.75:1, from about 5.75:1 to about 6:1, from about 6:1 to about 6.25:1, from about 6.25:1 to about 6.5:1, from about 6.5:1 to about 6.75:1, from about 6.75:1 to about 7:1, from about 7:1 to about 7.25:1, from about 7.25:1 to about 7.5:1, from about 7.5:1 to about 7.75:1, from about 7.75:1 to about 8:1, from about 8:1 to about 8.25:1, from about 8.25:1 to about 8.5:1, from about 8.5:1 to about 8.75:1, from about 8.75:1 to about 9:1, from about 9:1 to about 9.25:1, from about 9.25:1 to about 9.5:1, from about 9.5:1 to about 9.75:1, or from about 9.75:1 to about 10:1 by weight.

As noted above, the non-crystalline cannabinoid oil of the present disclosure can comprise up to 100% cannabinoid content. Therefore, in some embodiments, the Cannabinoid Products of the present disclosure have a cannabinoid to colloidal silicon dioxide particles ratio of about 0.5:1, about 0.6:1, about 0.7:1, about 0.75:1, about 0.8:1, about 0.9:1, about 1:1, about 1.1:1, about 1.2:1, about 1.25:1, about 1.3:1, about 1.4:1, about 1.5:1, about 1.6:1, about 1.7:1, about 1.75:1, about 1.8:1, about 1.9:1, about 2:1, about 2.1:1, about 2.2:1, about 2.25:1, about 2.3:1, about 2.4:1, about 2.5:1, about 2.6:1, about 2.7:1, about 2.75:1, about 2.8:1, about 2.9:1, about 3:1, about 3.1:1, about 3.2:1, about 3.25:1, about 3.3:1, about 3.4:1, about 3.5:1, about 3.6:1, about 3.7:1, about 3.75:1, about 3.8:1, about 3.9:1, about 4:1, about 4.25:1, about 4.5:1, about 4.75:1, about 5:1, about 5.25:1, about 5.5:1, about 5.75:1, about 6:1, about 6.25:1, about 6.5:1, about 6.75:1, about 7:1, about 7.25:1, about 7.5:1, about 7.75:1, about 8:1, about 8.25:1, about 8.5:1, about 8.75:1, about 9:1, about 9.25:1, about 9.5:1, about 9.75:1, or about 10:1 by weight, including all values, subranges, and ranges therebetween.

In some embodiments, the Cannabinoid Products of the present disclosure have a cannabinoid to colloidal silicon dioxide particles ratio of at least about 0.5:1, about 0.6:1, about 0.7:1, about 0.75:1, about 0.8:1, about 0.9:1, about 1:1, about 1.1:1, about 1.2:1, about 1.25:1, about 1.3:1, about 1.4:1, about 1.5:1, about 1.6:1, about 1.7:1, about 1.75:1, about 1.8:1, about 1.9:1, about 2:1, about 2.1:1, about 2.2:1, about 2.25:1, about 2.3:1, about 2.4:1, about 2.5:1, about 2.6:1, about 2.7:1, about 2.75:1, about 2.8:1, about 2.9:1, about 3:1, about 3.1:1, about 3.2:1, about 3.25:1, about 3.3:1, about 3.4:1, about 3.5:1, about 3.6:1, about 3.7:1, about 3.75:1, about 3.8:1, about 3.9:1, about 4:1, about 4.25:1, about 4.5:1, about 4.75:1, about 5:1, about 5.25:1, about 5.5:1, about 5.75:1, about 6:1, about 6.25:1, about 6.5:1, about 6.75:1, about 7:1, about 7.25:1, about 7.5:1, about 7.75:1, about 8:1, about 8.25:1, about 8.5:1, about 8.75:1, about 9:1, about 9.25:1, about 9.5:1, about 9.75:1, or about 10:1 by weight, including all values, subranges, and ranges therebetween.

### Cannabinoid Daily Dose

In embodiments, the Cannabinoid Products of the current disclosure increase the dose potency within regulatory limits. In some instances, the FDA limits the amount of silicon dioxide that can be consumed. Consequently, the dosage of cannabinoids is limited to the FDA limitations on silicon dioxide consumption. Higher non-crystalline cannabinoid oil to colloidal silicon dioxide particle ratios are particularly desirable, as it reduces the amount of colloidal silicon dioxide administered to patients per unit dose of cannabinoid. Thus, formulations of the present disclosure deliver higher cannabinoid dosages without exceeding FDA limits on consumption of silicon dioxide. Moreover, the Cannabinoid Products of the current disclosure do not require a diluent. Use of a diluent inherently prohibits higher loading ratios, because the loaded oil is less diluted, and therefore, less potent.

In embodiments, the methods of the present disclosure provides a higher daily dose of cannabinoid within regulatory limits of silicon dioxide consumption than traditional solid (but not crystalline) formulations.

In embodiments, the daily dose comprises a tablet. In embodiments, the daily dose comprises at least 1 tablet, at least 2 tablets, at least 3 tablets, at least 4 tablets, at least 5 tablets, at least 6 tablets, at least 7 tablets, at least 8 tablets, at least 9 tablets, or at least 10 tablets.

In embodiments, the daily dose comprises a capsule. In embodiments, the daily dose comprises at least 1 capsule, at least 2 capsules, at least 3 capsules, at least 4 capsules, at least 5 capsules, at least 6 capsules, at least 7 capsules, at least 8 capsules, at least 9 capsules, or at least 10 capsules.

In embodiments, a single tablet comprises at least about 20 mg, about at least 30 mg, at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, or at least about 150 mg colloidal silicon dioxide particles comprising internal storage cavities, including all values, subranges, and ranges therebetween.

In embodiments, a single capsule comprises at least about 20 mg, about at least 30 mg, at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, or at least about 150 mg colloidal silicon dioxide particles comprising internal storage cavities, including all values, subranges, and ranges therebetween.

In embodiments, a single pill comprises from about 20 mg to about 30 mg, from about 30 mg to about 40 mg, from about 40 mg to about 50 mg, from about 50 mg to about 60 mg, from about 60 mg to about 70 mg, from about 70 mg to about 80 mg, from about 80 mg to about 90 mg, from about 90 mg to about 100 mg, from about 100 mg to about 110 mg, from about 110 mg to about 120 mg, from about 120 mg to about 130 mg, from about 130 mg to about 140 mg, or from about 140 mg to about 150 mg colloidal silicon dioxide particles comprising internal storage cavities.

In embodiments, the daily dose comprises at least about 0.1 mg/kg/day, at least about 0.2 mg/kg/day, at least about 0.3 mg/kg/day, at least about 0.4 mg/kg/day, at least about 0.5 mg/kg/day, at least about 0.6 mg/kg/day, at least about 0.7 mg/kg/day, at least about 0.8 mg/kg/day, at least about 0.9 mg/kg/day, at least about 1 mg/kg/day, at least about 2 mg/kg/day, at least about 3 mg/kg/day, at least about 4 mg/kg/day, at least about 5 mg/kg/day, at least about 6 mg/kg/day, at least about 7 mg/kg/day, at least about 8 mg/kg/day, at least about 9 mg/kg/day, at least about 10 mg/kg/day, at least about 11 mg/kg/day, at least about 12 mg/kg/day, at least about 13 mg/kg/day, at least about 14 mg/kg/day, at least about 15 mg/kg/day, at least about 16 mg/kg/day, at least about 17 mg/kg/day, at least about 18 mg/kg/day, at least about 19 mg/kg/day, at least about 20 mg/kg/day, at least about 21 mg/kg/day, at least about 22 mg/kg/day, at least about 23 mg/kg/day, at least about 24 mg/kg/day, at least about 25 mg/kg/day, at least about 26 mg/kg/day, at least about 27 mg/kg/day, at least about 28 mg/kg/day, at least about 29 mg/kg/day, or at least about 30 mg/kg/day, including all values, subranges, and ranges therebetween.

In embodiments, the daily dose comprises from about 0.1 mg/kg/day to about 0.2 mg/kg/day, about 0.2 mg/kg/day to about 0.3 mg/kg/day, about 0.3 mg/kg/day to about 0.4 mg/kg/day, about 0.4 mg/kg/day to about 0.5 mg/kg/day, about 0.5 mg/kg/day to about 0.6 mg/kg/day, about 0.6 mg/kg/day to about 0.7 mg/kg/day, about 0.7 mg/kg/day to about 0.8 mg/kg/day, about 0.8 mg/kg/day to about 0.9 mg/kg/day, about 0.9 mg/kg/day to about 1 mg/kg/day, about 1 mg/kg/day to about 2 mg/kg/day, about 2 mg/kg/day to about 3 mg/kg/day, about 3 mg/kg/day to about 4 mg/kg/day, about 4 mg/kg/day to about 5 mg/kg/day, about 5 mg/kg/day to about 6 mg/kg/day, about 6 mg/kg/day to about 7 mg/kg/day, about 7 mg/kg/day to about 8 mg/kg/day, about 8 mg/kg/day to about 9 mg/kg/day, about 9 mg/kg/day to about 10 mg/kg/day, about 10 mg/kg/day to about 11 mg/kg/day, about 11 mg/kg/day to about 12 mg/kg/day, about 12 mg/kg/day to about 13 mg/kg/day, about 13 mg/kg/day to about 14 mg/kg/day, about 14 mg/kg/day to about 15 mg/kg/day, about 15 mg/kg/day to about 16 mg/kg/day, about 16 mg/kg/day to about 17 mg/kg/day, about 17 mg/kg/day to about 18 mg/kg/day, about 18 mg/kg/day to about 19 mg/kg/day, about 19 mg/kg/day to about 20 mg/kg/day, about 20 mg/kg/day to about 21 mg/kg/day, about 21 mg/kg/day to about 22 mg/kg/day, about 22 mg/kg/day to about 23 mg/kg/day, about 23 mg/kg/day to about 24 mg/kg/day, about 24 mg/kg/day to about 25 mg/kg/day, about 25 mg/kg/day to about 26 mg/kg/day, about 26 mg/kg/day to about 27 mg/kg/day, about 27 mg/kg/day to about 28 mg/kg/day, about 28 mg/kg/day to about 29 mg/kg/day, or from about 29 mg/kg/day to about 30 mg/kg/day.

In some embodiments the Cannabinoid Product is a daily dose within FDA silicon dioxide particle limits described and incorporated by reference herein, further comprising about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1010 mg, about 1020 mg, about 1030 mg, about 1040 mg, about 1050 mg, about 1060 mg, about 1070 mg, about 1080 mg, about 1090 mg, about 1100 mg, about 1110 mg, about 1120 mg, about 1130 mg, about 1140 mg, about 1150 mg, about 1160 mg, about 1170 mg, about 1180 mg, about 1190 mg, about 1200 mg, about 1210 mg, about 1220 mg, about 1230 mg, about 1240 mg, about 1250 mg, about 1260 mg, about 1270 mg, about 1280 mg, about 1290 mg, about 1300 mg, about 1310 mg, about 1320 mg, about 1330 mg, about 1340 mg, about 1350 mg, about 1360 mg, about 1370 mg, about 1380 mg, about 1390 mg, about 1400 mg, about 1410 mg, about 1420 mg, about 1430 mg, about 1440 mg, about 1450 mg, about 1460 mg, about 1470 mg, about 1480 mg, about 1490 mg, about 1500 mg, about 1510 mg, about 1520 mg, about 1530 mg, about 1540 mg, about 1550 mg, about 1560 mg, about 1570 mg, about 1580 mg, about 1590 mg, about 1600 mg, about 1610 mg, about 1620 mg, about 1630 mg, about 1640 mg, about 1650 mg, about 1660 mg, about 1670 mg, about 1680 mg, about 1690 mg, about 1700 mg, about 1710 mg, about 1720 mg, about 1730 mg, about 1740 mg, about 1750 mg, about 1760 mg, about 1770 mg, about 1780 mg, about 1790 mg, about 1800 mg, about 1810 mg, about 1820 mg, about 1830 mg, about 1840 mg, about 1850 mg, about 1860 mg, about 1870 mg, about 1880 mg, about 1890 mg, about 1900 mg, about 1910 mg, about 1920 mg, about 1930 mg, about 1940 mg, about 1950 mg, about 1960 mg, about 1970 mg, about 1980 mg, about 1990 mg, or about 2000 cannabinoid (e.g., mg of any cannabinoid of the present disclosure, and combinations thereof), including all ranges and subranges therein.

### Further Properties

In some embodiments, the Cannabinoid Products of the present disclosure are solid. In some embodiments, the cannabinoid products of the present disclosure are in the form of free flowing powder. In other embodiments, the Cannabinoid Products of the present disclosure are in shapes (e.g., compressed into tablets). In embodiments, the Cannabinoid Products of the current disclosure possess unique mass, diameter, shape, hardiness, compression force, and disintegration properties.

### Mass

In embodiments, the mass of the Cannabinoid Product ranges from about 100 mg to about 800 mg. In embodiments, the mass of the Cannabinoid Product ranges from about 100 mg to about 200 mg. In embodiments, the mass of the Cannabinoid Product ranges from about 500 mg to about 600 mg. In embodiments, the mass of the Cannabinoid Product ranges from about 700 mg to about 800 mg. In embodiments, the Cannabinoid Product described herein are about 103 mg. In some embodiments each discrete Cannabinoid Product (e.g., pill, tablet, capsule) is about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1010 mg, about 1020 mg, about 1030 mg, about 1040 mg, about 1050 mg, about 1060 mg, about 1070 mg, about 1080 mg, about 1090 mg, about 1100 mg, about 1110 mg, about 1120 mg, about 1130 mg, about 1140 mg, about 1150 mg, about 1160 mg, about 1170 mg, about 1180 mg, about 1190 mg, about 1200 mg, about 1210 mg, about 1220 mg, about 1230 mg, about 1240 mg, about 1250 mg, about 1260 mg, about 1270 mg, about 1280 mg, about 1290 mg, about 1300 mg, about 1310 mg, about 1320 mg, about 1330 mg, about 1340 mg, about 1350 mg, about 1360 mg, about 1370 mg, about 1380 mg, about 1390 mg, about 1400 mg, about 1410 mg, about 1420 mg, about 1430 mg, about 1440 mg, about 1450 mg, about 1460 mg, about 1470 mg, about 1480 mg, about 1490 mg, or about 1500 mg in weight, including all ranges and subranges therebetween.

### Diameter

In embodiments, the Cannabinoid Product diameter ranges from about 4 mm to about 25 mm. In embodiments, the tablet diameter is about 4 mm, about 4.1 mm, about 4.2 mm, about 4.3 mm, about 4.4 mm, about 4.5 mm, about 4.6 mm, about 4.7 mm, about 4.8 mm, about 4.9 mm, about 5 mm, about 5.1 mm, about 5.2 mm, about 5.3 mm, about 5.4 mm, about 5.5 mm, about 5.6 mm, about 5.7 mm, about 5.8 mm, about 5.9 mm, about 6 mm, about 6.1 mm, about 6.2 mm, about 6.3 mm, about 6.4 mm, about 6.5 mm, about 6.6 mm, about 6.7 mm, about 6.8 mm, about 6.9 mm, about 7 mm, about 7.1 mm, about 7.2 mm, about 7.3 mm, about 7.4 mm, about 7.5 mm, about 7.6 mm, about 7.7 mm, about 7.8 mm, about 7.9 mm, about 8 mm, about 8.1 mm, about 8.2 mm, about 8.3 mm, about 8.4 mm, about 8.5 mm, about 8.6 mm, about 8.7 mm, about 8.8 mm, about 8.9 mm, about 9 mm, about 9.1 mm, about 9.2 mm, about 9.3 mm, about 9.4 mm, about 9.5 mm, about 9.6 mm, about 9.7 mm, about 9.8 mm, about 9.9 mm, about 10 mm, about 10.1 mm, about 10.2 mm, about 10.3 mm, about 10.4 mm, about 10.5 mm, about 10.6 mm, about 10.7 mm, about 10.8 mm, about 10.9 mm, about 11 mm, about 11.1 mm, about 11.2 mm, about 11.3 mm, about 11.4 mm, about 11.5 mm, about 11.6 mm, about 11.7 mm, about 11.8 mm, about 11.9 mm, about 12 mm, about 12.1 mm, about 12.2 mm, about 12.3 mm, about 12.4 mm, about 12.5 mm, about 12.6 mm, about 12.7 mm, about 12.8 mm, about 12.9 mm, about 13 mm, about 13.1 mm, about 13.2 mm, about 13.3 mm, about 13.4 mm, about 13.5 mm, about 13.6 mm, about 13.7 mm, about 13.8 mm, about 13.9 mm, about 14 mm, about 14.1 mm, about 14.2 mm, about 14.3 mm, about 14.4 mm, about 14.5 mm, about 14.6 mm, about 14.7 mm, about 14.8 mm, about 14.9 mm, about 15 mm, about 15.1 mm, about 15.2 mm, about 15.3 mm, about 15.4 mm, about 15.5 mm, about 15.6 mm, about 15.7 mm, about 15.8 mm, about 15.9 mm, about 16 mm, about 16.1 mm, about 16.2 mm, about 16.3 mm, about 16.4 mm, about 16.5 mm, about 16.6 mm, about 16.7 mm, about 16.8 mm, about 16.9 mm, about 17 mm, about 17.1 mm, about 17.2 mm, about 17.3 mm, about 17.4 mm, about 17.5 mm, about 17.6 mm, about 17.7 mm, about 17.8 mm, about 17.9 mm, about 18 mm, about 18.1 mm, about 18.2 mm, about 18.3 mm, about 18.4 mm, about 18.5 mm, about 18.6 mm, about 18.7 mm, about 18.8 mm, about 18.9 mm, about 19 mm, about 19.1 mm, about 19.2 mm, about 19.3 mm, about 19.4 mm, about 19.5 mm, about 19.6 mm, about 19.7 mm, about 19.8 mm, about 19.9 mm, about 20 mm, about 20.1 mm, about 20.2 mm, about 20.3 mm, about 20.4 mm, about 20.5 mm, about 20.6 mm, about 20.7 mm, about 20.8 mm, about 20.9 mm, about 21 mm, about 21.1 mm, about 21.2 mm, about 21.3 mm, about 21.4 mm, about 21.5 mm, about 21.6 mm, about 21.7 mm, about 21.8 mm, about 21.9 mm, about 22 mm, about 22.1 mm, about 22.2 mm, about 22.3 mm, about 22.4 mm, about 22.5 mm, about 22.6 mm, about 22.7 mm, about 22.8 mm, about 22.9 mm, about 23 mm, about 23.1 mm, about 23.2 mm, about 23.3 mm, about 23.4 mm, about 23.5 mm, about 23.6 mm, about 23.7 mm, about 23.8 mm, about 23.9 mm, about 24 mm, about 24.1 mm, about 24.2 mm, about 24.3 mm, about 24.4 mm, about 24.5 mm, about 24.6 mm, about 24.7 mm, about 24.8 mm, about 24.9 mm, or about 25 mm, including all subranges, ranges, and values therebetween. In some embodiments, the diameter of the Cannabinoid Product is based on the diameter on the die used to shape it. Thus, in embodiments, the diameter of the die is about 4 mm, about 4.1 mm, about 4.2 mm, In embodiments, the diameter of the die is about 6.35 mm (about 0.25 inches). In embodiments, the diameter of the die is about 7.94 mm (about 0.3125 inches). In embodiments, the diameter of the die is about 7 mm. In embodiments, the diameter of the die is about 13 mm.

### Shape

In embodiments, the Cannabinoid Product shape is selected from flat faced, shallow convey, normal convex, deep convex, ball or pill, flat beveled edge, double radius, bevel and convex, dimple, ring, rim, capsule, oval, ellipse, square, triangle, pentagon, hexagon, heptagon, octagon, diamond, pillow, barrel, rectangle, almond, arrowhead, bullet, half moon, shield, heart, star, or round. In embodiments, the Cannabinoid Product shape is round. In embodiments, the Cannabinoid Product shape is round with a flat face bevel edge. In embodiments, the Cannabinoid Product shape is round and standard convex.

### Hardness

In embodiments, the Cannabinoid Product has a hardness that ranges from 50 Newtons (N) to about 150 N. In embodiments, the Cannabinoid Product has a hardness that ranges from 50 N to about 100 N. In embodiments, the Cannabinoid Product has a hardness that ranges from 100 N to about 150 N. In embodiments, the Cannabinoid Product has a hardness that ranges from 60 N to about 70 N. For example, in embodiments, the Cannabinoid Product has a hardness of about 50 N, about 51 N, about 52 N, about 53 N, about 54 N, about 55 N, about 56 N, about 57 N, about 58 N, about 59 N, about 60 N, about 61 N, about 62 N, about 63 N, about 64 N, about 65 N, about 66 N, about 67 N, about 68 N, about 69 N, about 70 N, about 71 N, about 72 N, about 73 N, about 74 N, about 75 N, about 76 N, about 77 N, about 78 N, about 79 N, about 80 N, about 81 N, about 82 N, about 83 N, about 84 N, about 85 N, about 86 N, about 87 N, about 88 N, about 89 N, about 90 N, about 91 N, about 92 N, about 93 N, about 94 N, about 95 N, about 96 N, about 97 N, about 98 N, about 99 N, about 100 N, about 101 N, about 102 N, about 103 N, about 104 N, about 105 N, about 106 N, about 107 N, about 108 N, about 109 N, about 110 N, about 111 N, about 112 N, about 113 N, about 114 N, about 115 N, about 116 N, about 117 N, about 118 N, about 119 N, about 120 N, about 121 N, about 122 N, about 123 N, about 124 N, about 125 N, about 126 N, about 127 N, about 128 N, about 129 N, about 130 N, about 131 N, about 132 N, about 133 N, about 134 N, about 135 N, about 136 N, about 137 N, about 138 N, about 139 N, about 140 N, about 141 N, about 142 N, about 143 N, about 144 N, about 145 N, about 146 N, about 147 N, about 148 N, about 149 N, or about 150 N, including all values, subranges, and ranges therebetween. In embodiments, the Cannabinoid Product has a hardness of about 50 N, at least about 51 N, at least about 52 N, at least about 53 N, at least about 54 N, at least about 55 N, at least about 56 N, at least about 57 N, at least about 58 N, at least about 59 N, at least about 60 N, at least about 61 N, at least about 62 N, at least about 63 N, at least about 64 N, at least about 65 N, at least about 66 N, at least about 67 N, at least about 68 N, at least about 69 N, at least about 70 N, at least about 71 N, at least about 72 N, at least about 73 N, at least about 74 N, at least about 75 N, at least about 76 N, at least about 77 N, at least about 78 N, at least about 79 N, at least about 80 N, at least about 81 N, at least about 82 N, at least about 83 N, at least about 84 N, at least about 85 N, at least about 86 N, at least about 87 N, at least about 88 N, at least about 89 N, at least about 90 N, at least about 91 N, at least about 92 N, at least about 93 N, at least about 94 N, at least about 95 N, at least about 96 N, at least about 97 N, at least about 98 N, at least about 99 N, at least about 100 N, at least about 101 N, at least about 102 N, at least about 103 N, at least about 104 N, at least about 105 N, at least about 106 N, at least about 107 N, at least about 108 N, at least about 109 N, at least about 110 N, at least about 111 N, at least about 112 N, at least about 113 N, at least about 114 N, at least about 115 N, at least about 116 N, at least about 117 N, at least about 118 N, at least about 119 N, at least about 120 N, at least about 121 N, at least about 122 N, at least about 123 N, at least about 124 N, at least about 125 N, at least about 126 N, at least about 127 N, at least about 128 N, at least about 129 N, at least about 130 N, at least about 131 N, at least about 132 N, at least about 133 N, at least about 134 N, at least about 135 N, at least about 136 N, at least about 137 N, at least about 138 N, at least about 139 N, at least about 140 N, at least about 141 N, at least about 142 N, at least about 143 N, at least about 144 N, at least about 145 N, at least about 146 N, at least about 147 N, at least about 148 N, at least about 149 N, or at least about 150 N. In embodiments, large Cannabinoid Products (e.g., a tablet with a mass of 500 mg or larger) have a hardness from about 50 N to about 150 N. In embodiments, small Cannabinoid Products (e.g., a tablet with a mass of from about 100 mg to about 300 mg) have a hardness from about 50 N to about 100 N.

### Compression Force

In embodiments, the Cannabinoid Product has a compression force ranging from about 1 kilonewton (kN) to about 30 kN. In embodiments, the Cannabinoid Product has a compression force ranging from about 5 kN to about 20 kN. In embodiments, the Cannabinoid Product has a compression force ranging from about 10 kN to about 16 kN. For example, the compression force of a Cannabinoid Product is about 1 kN, about 2 kN, about 3 kN, about 4 kN, about 5 kN, about 6 kN, about 7 kN, about 8 kN, about 9 kN, about 10 kN, about 11 kN, about 12 kN, about 13 kN, about 14 kN, about 15 kN, about 16 kN, about 17 kN, about 18 kN, about 19 kN, about 20 kN, about 21 kN, about 22 kN, about 23 kN, about 24 kN, about 25 kN, about 26 kN, about 27 kN, about 28 kN, about 29 kN, or about 30 kN, including all values, subranges, and ranges therebetween. In embodiments, the compression force of a Cannabinoid Product is at least about 1 kN, at least about 2 kN, at least about 3 kN, at least about 4 kN, at least about 5 kN, at least about 6 kN, at least about 7 kN, at least about 8 kN, at least about 9 kN, at least about 10 kN, at least about 11 kN, at least about 12 kN, at least about 13 kN, at least about 14 kN, at least about 15 kN, at least about 16 kN, at least about 17 kN, at least about 18 kN, at least about 19 kN, at least about 20 kN, at least about 21 kN, at least about 22 kN, at least about 23 kN, at least about 24 kN, at least about 25 kN, at least about 26 kN, at least about 27 kN, at least about 28 kN, at least about 29 kN, or at least about 30 kN.

### Disintegration

In embodiments, the Cannabinoid Product substantially disintegrates in from about 10 sec to about 10 min. For example, in embodiments, the tablet substantially disintegrates in within about 10 sec, about 15 sec, about 20 sec, about 25 sec, about 30 sec, about 35 sec, about 40 sec, about 45 sec, about 50 sec, about 55 sec, about 60 sec, about 70 sec, about 80 sec, about 90 sec, about 2 min, about 2.5 min, about 3 min, about 3.5 min, about 4 min, about 4.5 min, about 5 min, about 5.5 min, about 6 min, about 6.5 min, about 7 min, about 7.5 min, about 8 min, about 8.5 min, about 9 min, about 9.5 min, or about 10 min including all values, subranges, and ranges therebetween. In embodiments, the tablet substantially disintegrates in 2% sodium lauryl sulfate in less than 5 minutes, less than 4 min, less than 3 min, less than 2 min, less than 1 min, or less than 30 sec.

In embodiments, the Cannabinoid Product has a Percentage Dissolved at 60 minutes of at least about 60%, at least about 65%, at least about 70%, at least about 75%, at least about 80%, at least about 85%, at least about 90%, at least about 95%, or more in a solvent. In embodiments, the solvent is 1% w/v Tween 80 (polysorbate 80) solution, fed state simulated intestinal fluid (“FeSSIF,” contains about 15 mM taurocholate, 3.75 mM phospholipids, 319 mM sodium ions, 203 mM chloride, and 144 mM acetic acid), fasted state simulated intestinal fluid (“FaSSIF,” contains 3 mM taurocholate, 0.75 mM phospholipids, 148 mM sodium ions, 106 mM chloride, and 29 mM phosphate ions).

In embodiments, a Cannabinoid Product containing non-crystalline cannabinoid oil and colloidal silicon dioxide particles described herein has a Percentage Dissolved at 60 minutes, which is improved relative to a tablet containing a Cannabinoid Product that contains non-heated, non-crystalline cannabinoid oil. In embodiments, the Percentage Dissolved at 60 minutes is improved by about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, about 9, about 9.1, about 9.2, about 9.3, about 9.4, about 9.5, about 9.6, about 9.7, about 9.8, about 9.9, about 10, or more fold, including all ranges and subranges therebetween.

In embodiments, the Cannabinoid Product is an orodispersable tablet or immediate release tablet.

### IV. Methods of Making Cannabinoid Products

Provided herein are methods of making the Cannabinoid Products. In each of the methods for making Cannabinoid Products described herein, the initial step comprises heating cannabinoid oil, which ensures that it is non-crystalline cannabinoid oil. The second step comprises contacting the heated non-crystalline cannabinoid oil with the colloidal silicon dioxide particles. In embodiments, the non-crystalline cannabinoid oil is added to the colloidal silicon dioxide particles. In embodiments, the colloidal silicon dioxide particles are added to the non-crystalline cannabinoid oil. In some embodiments the non-crystalline cannabinoid oil and colloidal silicon dioxide particles are mixed in the absence of other ingredients, to ensure that the non-crystalline cannabinoid oil is loaded into the particles, and not merely suspended with other ingredients.

In embodiments, provided herein is a method of loading a non-crystalline cannabinoid oil into a plurality of colloidal silicon dioxide particles comprising internal storage cavities, said method comprising the steps of: (a) providing a cannabinoid oil; (b) heating the cannabinoid oil, thereby producing non-crystalline cannabinoid oil; (c) providing a plurality of colloidal silicon dioxide particles comprising internal storage cavities; and (d) contacting the non-crystalline cannabinoid oil with the plurality colloidal silicon dioxide particles comprising internal storage cavities. Optionally, all of the non-crystalline cannabinoid oil is substantially loaded into the colloidal silicon dioxide particles comprising internal storage cavities.

In embodiments, provided herein is a method of making a solid Cannabinoid Product, said method comprising the steps of: (a) providing a cannabinoid oil; (b) heating the cannabinoid oil, thereby producing non-crystalline cannabinoid oil; (c) providing a plurality of colloidal silicon dioxide particles comprising internal storage cavities; (d) mixing the non-crystalline cannabinoid oil and colloidal silicon dioxide particles comprising internal storage cavities; (e) adding a filler and disintegrant to the mixture of non-crystalline cannabinoid oil and colloidal silicon dioxide particles comprising internal storage cavities; and (f) adding a lubricant.

In embodiments, provided herein is a method of making a Cannabinoid Product, said method comprising the steps of: (a) providing a cannabinoid oil; (b) heating the cannabinoid oil, thereby producing a non-crystalline cannabinoid oil; (c) providing a plurality of colloidal silicon dioxide particles comprising internal storage cavities; (d) mixing the non-crystalline cannabinoid oil and colloidal silicon dioxide particles comprising internal storage cavities; and (e) adding a lubricant.

### Heating

Applicants have discovered that heated non-crystalline cannabinoid oil has improved loading when compared to non-heated, non-crystalline cannabinoid oil. Heating further occurs before adding any filler to ensure the oil is substantially loaded. Loading of non-crystalline cannabinoid oil can be directly assessed (e.g., via microscopy), or can be inferred from stability o the Cannabinoid Product (e.g., maintaining white solid properties, instead of leaking out cannabinoid), or its dissolution properties (e.g., linear dissolution in the medias described in this disclosure). Additional information regarding loading of cannabinoid oil into colloidal silicon dioxide particles is provided in later sections of this document.

In embodiments, the initial step comprises heating cannabinoid oil. In embodiments, the non-crystalline cannabinoid oil comprises a cannabinoid isolate and/or terpenes.

In embodiments, the methods of creating a Cannabinoid Product comprise heating the cannabinoid oil. In embodiments, the methods of creating a Cannabinoid Product comprise heating a composition comprising the cannabinoid oil and colloidal silicon dioxide particles. In embodiments, the non-crystalline cannabinoid oil is heated before the oil is mixed with the colloidal silicon dioxide particles. In embodiments, the non-crystalline cannabinoid oil is heated to at least 30° C., at least 40° C., at least 50° C., at least 60° C., or at least 70° C. In embodiments, the non-crystalline cannabinoid oil is heated to about 60° C. In embodiments, the non-crystalline cannabinoid oil is heated to a temperature ranging from about 30° C. to about 70° C., including all values, ranges, and subranges therebetween. In embodiments, the non-crystalline cannabinoid oil is heated to a temperature ranging from about 30° C. to about 65° C., including all values, ranges, and subranges therebetween. In embodiments, the non-crystalline cannabinoid oil is heated to a temperature ranging from about 30° C. to about 60° C., including all values, ranges, and subranges therebetween. In embodiments, the non-crystalline cannabinoid oil is heated to about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., or about 100° C., including all ranges and subranges therebetween. In embodiments, the non-crystalline cannabinoid oil is not heated to a temperature higher than about 65° C. In embodiments, the non-crystalline cannabinoid oil is not heated to a temperature higher than about 70° C. Without being bound by theory, a non-crystalline cannabinoid oil is not heated to a temperature above 70° C. because temperatures above 70° C. can degrade cannabinoids. In some embodiments, the heated cannabinoid oil removes any crystals that may have formed during storage (produces non-crystalline cannabinoid oil), and further aids in the loading of the non-crystalline cannabinoid oil into the colloidal silicon dioxide particles.

### Mixing

In embodiments, the second step comprises mixing non-crystalline cannabinoid oil and colloidal silicon dioxide particles.

In embodiments, a mixing step of the methods described herein comprises high-shear mixing using a high shear mixer. The term “high shear mixer” refers to an apparatus that disperses a first ingredient (e.g., a non-crystalline cannabinoid oil) into a second ingredient (e.g., colloidal silicon dioxide particles) using mechanical agitation. In embodiments, the high shear mixer comprises a rotor or an impeller with a stationary component called a stator. Non-limiting examples of high-shear mixers include batch high shear mixers, inline high shear mixers, ultra-high shear inline mixers, grinding mills, and grinders. In embodiments, the high-shear mixer is a coffee grinder.

In embodiments, the high shear mixer operates at a speed of from 500 revolutions per minutes (rpm) to about 5000 rpm. In embodiments, the high shear mixer operates at a speed of from 1000 rpm to about 2000 rpm. For example, in embodiments, the speed of the high shear mixer is about 500 rpm, about 600 rpm, about 700 rpm, about 800 rpm, about 900 rpm, about 1000 rpm, about 1100 rpm, about 1200 rpm, about 1300 rpm, about 1400 rpm, about 1500 rpm, about 1600 rpm, about 1700 rpm, about 1800 rpm, about 1900 rpm, about 2000 rpm, about 2100 rpm, about 2200 rpm, about 2300 rpm, about 2400 rpm, about 2500 rpm, about 2600 rpm, about 2700 rpm, about 2800 rpm, about 2900 rpm, about 3000 rpm, about 3100 rpm, about 3200 rpm, about 3300 rpm, about 3400 rpm, about 3500 rpm, about 3600 rpm, about 3700 rpm, about 3800 rpm, about 3900 rpm, about 4000 rpm, about 4100 rpm, about 4200 rpm, about 4300 rpm, about 4400 rpm, about 4500 rpm, about 4600 rpm, about 4700 rpm, about 4800 rpm, about 4900 rpm, or about 5000 rpm, including all values, subranges, and ranges therebetween.

In embodiments, high-shear mixing comprises placing a first ingredient and second ingredient in a high-shear mixer and pulsing the mixture. In embodiments, the mixture is exposed to a pulse that lasts for about 1 second, about 2 seconds, about 3 seconds, about 4 seconds, about 5 seconds, about 6 seconds, about 7 seconds, about 8 seconds, about 9 seconds, or about 10 seconds. In embodiments, the first ingredient and second ingredient are mixed in the high-shear mixer for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 pulses, including all values, subranges, and ranges therebetween.

In embodiments, the methods described herein comprise high-shear mixing for from about 1 second (sec) to about 1 minute (min). In embodiments, the methods described herein comprise high-shear mixing for from about 1 sec to about 5 min. In embodiments, the methods described herein comprise high-shear mixing for from about 1 sec to about 10 min. In embodiments, the methods described herein comprise high-shear mixing for from about 1 second to about 15 min. In embodiments, the methods described herein comprise high-shear mixing for from about 1 sec to about 20 min. In embodiments, the methods described herein comprise high-shear mixing for from about 1 sec to about 25 min. In embodiments, the methods described herein comprise high-shear mixing for from about 1 sec to about 30 min. In embodiments, the methods described herein comprise high-shear mixing for less than a minute, up to 20 min, at least 1 min, at least 5 min, at least 10 min, at least 15 min, or at least 20 min. For example, in embodiments, the methods described herein comprise high-shear mixing for about 1 sec, about 2 sec, about 3 sec, about 4 sec, about 5 sec, about 6 sec, about 7 sec, about 8 sec, about 9 sec, about 10 sec, about 15 sec, about 20 sec, about 25 sec, about 30 sec, about 35 sec, about 40 sec, about 45 sec, about 50 sec, about 55 sec, about 1 min, about 2 min, about 3 min, about 4 min, about 5 min, about 6 min, about 7 min, about 8 min, about 9 min, about 10 min, about 11 min, about 12 min, about 13 min, about 14 min, about 15 min, about 16 min, about 17 min, about 18 min, about 19 min, about 20 min, about 21 min, about 22 min, about 23 min, about 24 min, about 25 min, about 26 min, about 27 min, about 28 min, about 29 min, or about 30 min, including all values, subranges, and ranges therebetween.

In embodiments, the methods of making the Cannabinoid Products described herein comprise additional forms of mixing. Non-limiting examples of mixing include a stand mixing, bag-mixing, and v-blending. In embodiments, any combination of the ingredients within the Cannabinoid Products may be mixed using a mixing element selected from the group consisting of: spatulas, paddles, mechanical mixers, mechanical stirrers, stir bars, media dispensers, solid particles that aid in mixing the compounds (e.g., mixing balls), overhead mixers, static mixers, a v-blender, a bag-mixer, a stand-mixer, or a mortar and a pestle.

In embodiments, a composition comprising colloidal silicon dioxide particles and non-crystalline cannabinoid oil is mixed with one or more of a lubricant, a filler, a disintegrant, or a flavoring. In embodiments, mixing occurs from about 1 sec to 24 hours (h). For example, in embodiments, mixing occurs for about 1 sec, about 2 sec, about 3 sec, about 4 sec, about 5 sec, about 6 sec, about 7 sec, about 8 sec, about 9 sec, about 10 sec, about 15 sec, about 20 sec, about 25 sec, about 30 sec, about 35 sec, about 40 sec, about 45 sec, about 50 sec, about 55 sec, about 1 min, about 2 min, about 3 min, about 4 min, about 5 min, about 6 min, about 7 min, about 8 min, about 9 min, about 10 min, about 11 min, about 12 min, about 13 min, about 14 min, about 15 min, about 16 min, about 17 min, about 18 min, about 19 min, about 20 min, about 21 min, about 22 min, about 23 min, about 24 min, about 25 min, about 26 min, about 27 min, about 28 min, about 29 min, about 30 min, about 45 min, about 1 h, about 1.5 h, about 2 h, about 2.5 h, about 3 h, about 3.5 h, about 4 h, about 4.5 h, about 5 h, about 5.5 h, about 6 h, about 6.5 h, about 7 h, about 7.5 h, about 8 h, about 8.5 h, about 9 h, about 9.5 h, about 10 h, about 10.5 h, about 11 h, about 11.5 h, about 12 h, about 12.5 h, about 13 h, about 13.5 h, about 14 h, about 14.5 h, about 15 h, about 15.5 h, about 16 h, about 16.5 h, about 17 h, about 17.5 h, about 18 h, about 18.5 h, about 19 h, about 19.5 h, about 20 h, about 20.5 h, about 21 h, about 21.5 h, about 22 h, about 22.5 h, about 23 h, about 23.5 h, or about 24 h, including all subranges, ranges, and values therebetween.

In embodiments, colloidal silicon dioxide particles and non-crystalline cannabinoid oil are mixed in a high-shear mixer (e.g., a grinder). In embodiments, the composition is pulsed in the high-shear mixer for about 15 to about 30 pulses, wherein each pulse lasts one second. Optionally, the composition is grinded continuously for about 20 minutes until it is homogenous. In embodiments, a filler and disintegrant are added the to the composition containing cannabinoid oil and colloidal silicon dioxide particles. In embodiments, the filler and disintegrant are mixed with the composition containing cannabinoid oil and colloidal silicon dioxide particles for about 10 sec, about 15 sec, about 20 sec, about 25 sec, about 30 sec, about 35 sec, about 40 sec, about 45 sec, about 50 sec, about 55 sec, about 1 min, about 2 min, about 3 min, about 4 min, about 5 min, about 6 min, about 7 min, about 8 min, about 9 min, about 10 min, about 11 min, about 12 min, about 13 min, about 14 min, about 15 min, about 16 min, about 17 min, about 18 min, about 19 min, or about 20 min. In embodiments, the lubricant is and composition containing cannabinoid oil, colloidal silicon dioxide particles, filler, and disintegrant are mixed for less than 5 minutes, less than 4 minutes, less than 3 minutes, less than 2 minutes, or less than 1 minute.

In embodiments, non-crystalline cannabinoid oil, silicon dioxide particles, are mixed in a grinder until the composition is uniform. In embodiments, the composition containing non-crystalline cannabinoid oil, silicon dioxide particle is transferred to a mixing bowl, and optionally additional, fully melted cannabinoid isolate (e.g., CBD and/or CBG isolate) is added to the bowl, and the mixture is mixed for 1-20 minutes in a stand mixer. In embodiments, the mixture is mixed for about 5 min. In embodiments, the mixture is mixed for about 10 min. In embodiments, lubricant is added to the bowl. In embodiments, the lubricant is mixed with the non-crystalline cannabinoid oil, silicon dioxide particles, optionally one or more terpenes, and cannabinoid isolate for about 1 sec, about 5 sec, about 10 sec, about 15 sec, about 20 sec, about 25 sec, about 30 sec, about 35 sec, about 40 sec, about 45 sec, about 50 sec, about 55 sec, about 1 min, about 2 min, about 3 min, about 4 min, or about 5 min. In embodiments, the lubricant is mixed with the non-crystalline cannabinoid oil, silicon dioxide particles, optionally one or more terpenes, and optionally cannabinoid isolate for about 2 min. In embodiments, the lubricant is mixed with the non-crystalline cannabinoid oil, silicon dioxide particles, optionally one or more terpenes, and optionally cannabinoid isolate for about 1 min.

### Dilution

In embodiments, the non-crystalline cannabinoid oil is not diluted in any non-cannabis solvent (e.g., any dilution is due to natural liquid state at heated temperature, and/or the presence of remnant natural solvents (e.g., solvents from the cannabis plant) left from the extraction). For example, in some embodiments, non-crystalline cannabinoid oils may have small amounts of terpenes, which assist in keeping cannabinoids from crystallizing.

In some embodiments the cannabinoil oil is not diluted in any solvent. In embodiments, the methods of making Cannabinoid Products comprise diluting the non-crystalline cannabinoid oil in a solvent before the oil is mixed with the colloidal silicon dioxide particles. In embodiments, the solvent is selected from the group consisting of ethanol, methanol, propanol, olive oil, coconut oil, canola oil, flaxseed oil, avocado oil, sesame oil, canola oil, palm oil, safflower oil, soybean oil, corn oil, peanut oil, walnut oil, flaxseed oil, sunflower oil, palm oil, palm kernel oil, caproic acid, caprylic acid, hempseed oil, walnut oil, or medium-chain triglyceride (MCT) oil. In embodiments, the solvent is ethanol.

Loading Non-Crystalline Cannabinoid Oil into Colloidal Silicon Dioxide Particles

In embodiments, the Cannabinoid Products of the current disclosure exhibit enhanced loading into colloidal silicon dioxide particles. The inventors unexpectedly discovered that the Cannabinoid Products and methods of the current disclosure substantially loaded all heated non-crystalline cannabinoid oil into colloidal silicon dioxide particles. For example, the inventors used light microscopy to determine that relative percentage of heated non-crystalline cannabinoid oil that was loaded into the colloidal silicone dioxide particles. Heated non-crystalline cannabinoid oil was mostly present within the colloidal silicon dioxide particles, while a large percentage of non-heated cannabinoid oil was still in solution, outside of the particles.

Inventors further discovered that high cannabinoid oil to colloidal silicon dioxide particles made via traditional methods (without heat), would begin separating over time, yellowing, and returning to more liquid state. In contrast, Cannabinoid Products made according to the methods of the present disclosure with heated non-crystalline cannabinoid oil exhibited shelf stability, remaining solid white for tested intervals. These observations regarding the improper loading of traditional solid formulations may explain how the Cannabinoid Products of the current disclosure deliver significantly more cannabinoid oil and exhibit linear dissolution rates when compared to other products (See Examples 7-9).

In embodiments, the methods of the present disclosure load substantially all non-crystalline cannabinoid oil into colloidal silicon dioxide particles comprising internal storage cavities. In embodiments, substantially all non-crystalline cannabinoid oil is loaded into colloidal silicon dioxide particles comprising internal storage cavities in the Cannabinoid products of the current disclosure.

In embodiments, at least about 50%, at least about 55%, at least about 60%, at least about 65%, at least about 70%, at least about 75%, at least about 80%, at least about 85%, at least about 90%, at least about 95%, or about 100%, including all values, subranges, and ranges therebetween, of the non-crystalline cannabinoid oil is loaded into the colloidal silicon dioxide storage particles comprising internal storage cavities.

In embodiments, from about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% to about 100% of the non-crystalline cannabinoid oil is loaded into the colloidal silicon dioxide storage particles comprising internal storage cavities.

### Methods of Making Tablets

In embodiments, the Cannabinoid Products described herein are compressed into tablets using a tablet press. A tablet press uses a compression force to transform a powder, e.g., a Cannabinoid Product, into a tablet. Tablet presses comprise a tooling made of dies and punches. The die is a cavity where the powder for the tablet, e.g., the Cannabinoid Product is added. The die determines the diameter, size, and thickness of a tablet. The punches (upper and lower) compress the powder into tablets of various shapes within the die. The tablet tooling may be classified as “B,” “D,” “BB,” or “DB,” according to the diameter of the punch barrel and the outer diameter of the die. (Table 3).

TABLE 3
Types of Tablet Press Tooling

| Type of Tablet | Diameter of the Punch Barrel | Outer diameter of the die |
|---|---|---|
| Press Tooling | (mm) | (mm) |
| B | 19 | 30.16 |
| D | 25.4 | 38.10 |
| BB | 19.0 | 24.00 |
| DB | 25.4 | 30.16 |

In embodiments, the tablet press is a “single punch tablet press machine.” Single punch tablet press machines use a single set of station tooling (e.g., die and punches) to process a powder into the desired tablet size. In embodiments, the tablet press is a rotary tablet press. A rotary press has multiple sets of dies with lower and upper punches, which are in constant rotary motion. In embodiments, the rotary press is the GlobePharma Mini Press-II. The GlobePharma Mini Press-II has five stations of B tooling and five stations of D tooling. The GlobePharma Mini Press-II also has a gravity hopper and gravity feeder which allows Cannabinoid Product to flow into the die.

In embodiments, the diameter of the die (also referred to as “tablet diameter of tooling”) determines the tablet diameter. In embodiments, the diameter of the die ranges from 4 mm to about 25 mm. In embodiments, the diameter of the die is about 4 mm, about 4.1 mm, about 4.2 mm, about 4.3 mm, about 4.4 mm, about 4.5 mm, about 4.6 mm, about 4.7 mm, about 4.8 mm, about 4.9 mm, about 5 mm, about 5.1 mm, about 5.2 mm, about 5.3 mm, about 5.4 mm, about 5.5 mm, about 5.6 mm, about 5.7 mm, about 5.8 mm, about 5.9 mm, about 6 mm, about 6.1 mm, about 6.2 mm, about 6.3 mm, about 6.4 mm, about 6.5 mm, about 6.6 mm, about 6.7 mm, about 6.8 mm, about 6.9 mm, about 7 mm, about 7.1 mm, about 7.2 mm, about 7.3 mm, about 7.4 mm, about 7.5 mm, about 7.6 mm, about 7.7 mm, about 7.8 mm, about 7.9 mm, about 8 mm, about 8.1 mm, about 8.2 mm, about 8.3 mm, about 8.4 mm, about 8.5 mm, about 8.6 mm, about 8.7 mm, about 8.8 mm, about 8.9 mm, about 9 mm, about 9.1 mm, about 9.2 mm, about 9.3 mm, about 9.4 mm, about 9.5 mm, about 9.6 mm, about 9.7 mm, about 9.8 mm, about 9.9 mm, about 10 mm, about 10.1 mm, about 10.2 mm, about 10.3 mm, about 10.4 mm, about 10.5 mm, about 10.6 mm, about 10.7 mm, about 10.8 mm, about 10.9 mm, about 11 mm, about 11.1 mm, about 11.2 mm, about 11.3 mm, about 11.4 mm, about 11.5 mm, about 11.6 mm, about 11.7 mm, about 11.8 mm, about 11.9 mm, about 12 mm, about 12.1 mm, about 12.2 mm, about 12.3 mm, about 12.4 mm, about 12.5 mm, about 12.6 mm, about 12.7 mm, about 12.8 mm, about 12.9 mm, about 13 mm, about 13.1 mm, about 13.2 mm, about 13.3 mm, about 13.4 mm, about 13.5 mm, about 13.6 mm, about 13.7 mm, about 13.8 mm, about 13.9 mm, about 14 mm, about 14.1 mm, about 14.2 mm, about 14.3 mm, about 14.4 mm, about 14.5 mm, about 14.6 mm, about 14.7 mm, about 14.8 mm, about 14.9 mm, about 15 mm, about 15.1 mm, about 15.2 mm, about 15.3 mm, about 15.4 mm, about 15.5 mm, about 15.6 mm, about 15.7 mm, about 15.8 mm, about 15.9 mm, about 16 mm, about 16.1 mm, about 16.2 mm, about 16.3 mm, about 16.4 mm, about 16.5 mm, about 16.6 mm, about 16.7 mm, about 16.8 mm, about 16.9 mm, about 17 mm, about 17.1 mm, about 17.2 mm, about 17.3 mm, about 17.4 mm, about 17.5 mm, about 17.6 mm, about 17.7 mm, about 17.8 mm, about 17.9 mm, about 18 mm, about 18.1 mm, about 18.2 mm, about 18.3 mm, about 18.4 mm, about 18.5 mm, about 18.6 mm, about 18.7 mm, about 18.8 mm, about 18.9 mm, about 19 mm, about 19.1 mm, about 19.2 mm, about 19.3 mm, about 19.4 mm, about 19.5 mm, about 19.6 mm, about 19.7 mm, about 19.8 mm, about 19.9 mm, about 20 mm, about 20.1 mm, about 20.2 mm, about 20.3 mm, about 20.4 mm, about 20.5 mm, about 20.6 mm, about 20.7 mm, about 20.8 mm, about 20.9 mm, about 21 mm, about 21.1 mm, about 21.2 mm, about 21.3 mm, about 21.4 mm, about 21.5 mm, about 21.6 mm, about 21.7 mm, about 21.8 mm, about 21.9 mm, about 22 mm, about 22.1 mm, about 22.2 mm, about 22.3 mm, about 22.4 mm, about 22.5 mm, about 22.6 mm, about 22.7 mm, about 22.8 mm, about 22.9 mm, about 23 mm, about 23.1 mm, about 23.2 mm, about 23.3 mm, about 23.4 mm, about 23.5 mm, about 23.6 mm, about 23.7 mm, about 23.8 mm, about 23.9 mm, about 24 mm, about 24.1 mm, about 24.2 mm, about 24.3 mm, about 24.4 mm, about 24.5 mm, about 24.6 mm, about 24.7 mm, about 24.8 mm, about 24.9 mm, or about 25 mm. For example, in embodiments, the diameter of the die is about 4 mm, about 4.1 mm, about 4.2 mm, In embodiments, the diameter of the die is about 6.35 mm (about 0.25 inches). In embodiments, the diameter of the die is about 7.94 mm (about 0.3125 inches). In embodiments, the diameter of the die is about 7 mm. In embodiments, the diameter of the die is about 13 mm.

### Methods of Making Capsules

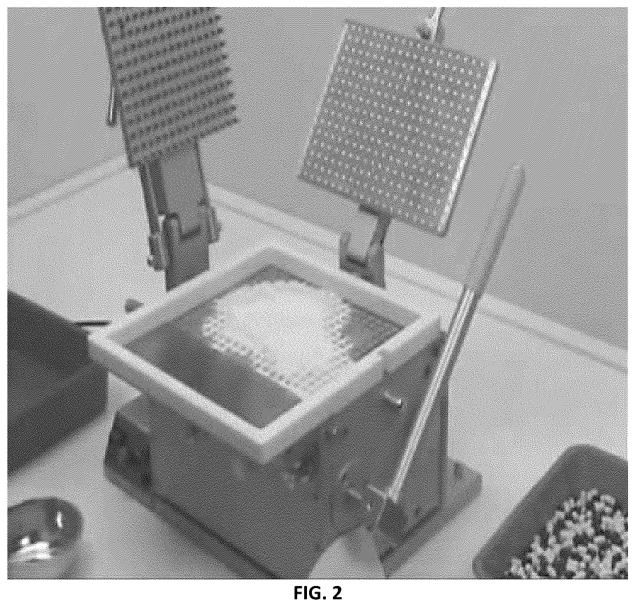

In embodiments, capsules comprising Cannabinoid Products described herein are created by filling the capsule shell with a Cannabinoid Product. In embodiments, a capsule filling machine is used to fill the capsule shells. For example, in embodiments, the capsules comprising the Cannabinoid Products described herein are created with the capsule filling machine of FIG. 2.

### EXAMPLES

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1—Stabilizing Cannabinoids in Colloidal Silicon Dioxide

The inventors conducted an initial evaluation of colloidal silicon dioxide as a stabilizer of non-crystalline cannabinoid oil. A composition containing a non-crystalline cannabinoid oil and colloidal silicon dioxide particles was prepared and compared against a composition lacking the ingredient.

A 250 g batch of the composition containing non-crystalline cannabinoid oil and colloidal silicon dioxide particles was prepared with the ingredients listed in Table 4.

Briefly, the cannabinoid oil was initially heated to 60° C. to lower the viscosity of the oil, and remove any crystals that may have formed during storage. The non-crystalline cannabinoid oil was added to the colloidal silicon dioxide, and the combination was homogenized using a dry, high shear mixing process. Microcrystalline cellulose and lubricant were subsequently added to the composition.

TABLE 4
Compositions

| | Capsule containing | | Capsule lacking | |
|---|---|---|---|---|
| | colloidal silicon | | colloidal silicon | |
| | dioxide | | dioxide | |
| | | (mg/ | | (mg/ |
| | (% w/w) | capsule) | (% w/w) | capsule) |
| Non-crystalline | 21.7 | 50 | 32.2 | 50 |
| cannabinoid oil from | | | | |
| heated Cannabidiol | | | | |
| (CBD) Isolate | | | | |
| Non-crystalline | 8.5 | 19.5 | 12.5 | 19.5 |
| cannabinoid oil from | | | | |
| heated Cannabigerol | | | | |
| (CBG) Isolate | | | | |
| Non-crystalline | 2.2 | 5.0 | 3.2 | 5 |
| cannabinoid oil (broad | | | | |
| spectrum) | | | | |
| Colloidal silicon dioxide | 32.4 | 74.5 | 0 | 0 |
| Microcrystalline cellulose | 34.8 | 80.0 | 51.5 | 80 |
| Sodium stearyl fumarate | 0.4 | 1.0 | 0.6 | 1 |

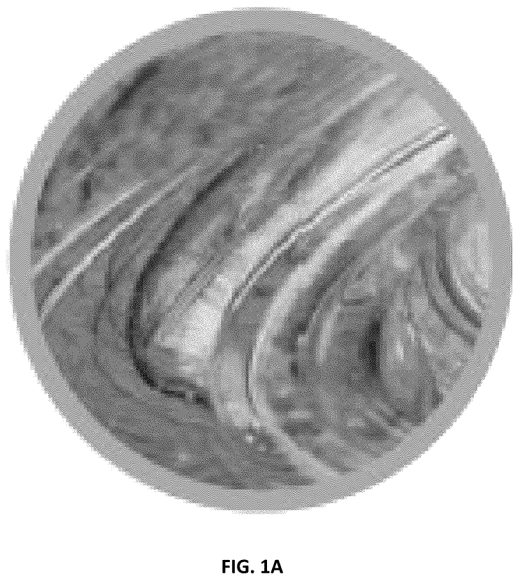

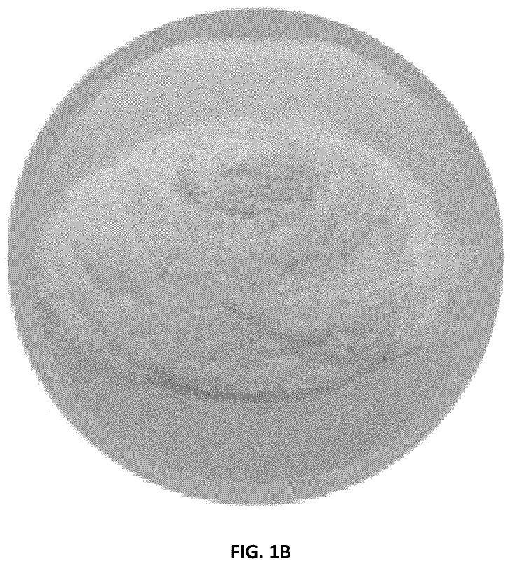

FIG. 1B shows the composition containing a non-crystalline cannabinoid oil and colloidal silicon dioxide. The composition forms a dry, non-sticky, powder, that can be dosed into pharmaceutical, homeopathic, dietary supplement, and/or recreational products. The non-crystalline cannabinoid oil is trapped in the structure of the silicon dioxide. In contrast, a composition containing a non-crystalline cannabinoid oil, but no colloidal silicon dioxide, does not form a dry powder (FIG. 1A). Rather, the non-crystalline cannabinoid oil separates from the powder bed (i.e., the CBG isolate, CBD isolate, colloidal silicon dioxide, microcrystalline cellulose, and sodium stearyl fumarate).

### Example 2—Dissolving Properties of Cannabinoid Oil and Colloidal Silicon Dioxide

Having confirmed colloidal silicon dioxide's ability to stabilize cannabinoid formulations, the inventors next turned to evaluating its dissolution properties. Capsules containing cannabinoid oil and colloidal silicon dioxide were prepared according to the recipes and methods described in Example 1. Each capsule contains 74.5 mg of active ingredients (i.e.; cannabinoid oil, including melted CBG isolate; and melted CBD isolate). Dissolution was measured using fiber optic dissolution probes (Pion Inc.'s RAINBOW® μDiss Profiler).

Capsules were submerged in either a i) fasted state simulated intestinal fluid (FaSSIF), or ii) a fed state simulated intestinal fluid (FeSSIF). The FaSSIF contains about 3 mM taurocholate, 0.75 mM phospholipids, 148 mM sodium ions, 106 mM chloride, and 29 mM phosphate ions, thereby modeling the conditions present in fasted small intestine gastrointestinal fluid. The pH of FaSSIF is about 6.5. FeSSIF contains about 15 mM taurocholate, 3.75 mM phospholipids, 319 mM sodium ions, 203 mM chloride, and 144 mM acetic acid. The pH of FeSSIF is about 5.0. FeSSIF contains similar osmolality and pH to fed intestinal fluid. A iii) control polysorbate 80 (TWEEN®80) solution was also tested in the experiment, to show dissolution in a simpler media. Table 5 below, summarizes the results from this experiment as percent dissolution within 60 minutes (higher value indicates greater dissolution).

TABLE 5
Dissolution of Capsules Containing or
Lacking Colloidal Silicon Dioxide

| Formulations | Control | FaSSIF | FeSSIF |
|---|---|---|---|
| Capsules containing colloidal silicon | 36.5% | 18.1% | 42.3% |
| dioxide and non-crystalline | | | |
| cannabinoid oil | | | |
| Capsules containing non-crystalline | 8.7% | 4.1% | 17.1% |
| cannabinoid oil without colloidal | | | |
| silicon dioxide | | | |
| Dissolution Improvement Factor | 4.2X | 4.4X | 2.5X |
| (DIF) | | | |

Table 5 shows that capsules containing compositions containing colloidal silicon dioxide particles dissolved more quickly than capsules containing compositions lacking colloidal silicon dioxide particles under all conditions. Optimal dissolution was achieved by the colloidal silicon dioxide particles and non-crystalline cannabinoid oil combination under the FeSSIF. The formulation containing colloidal silicon dioxide particles however exhibited the greatest dissolution improvement factor in the FaSSIF, where the percent dissolved at 60 minutes is 4.4 times higher than oil without the colloidal silicon dioxide.

### Example 3—Tablets Containing Non-Crystalline Cannabinoid Oil

Examples 1 and 2 above demonstrated the stabilization and dissolution benefits of the combination of colloidal silicon dioxide particles and non-crystalline cannabinoid oil. This example describes the development and formulation of commercial-grade tablets. Specifically, it was discovered that additional ingredients could further improve the properties of cannabinoid-based tablets by providing more desirable hardness and dissolution properties. Assessment of each of the tested formulations is described in more detail below and in Example 4.

Tablets comprising compositions containing a non-crystalline cannabinoid oil, colloidal silicon dioxide, a filler (i.e., microcrystalline cellulose or mannitol), a disintegrant (i.e. sodium starch glycolate or croscarmellose sodium), and a lubricant (i.e. sodium stearyl fumarate) were prepared according to the methods described below. Tables 6, 7, and 8 show the components of each composition. Each table contains the percent by weight of each component of the compositions. Each composition in Table 5 formed a suitable tablet with desirable commercial properties. The compositions in Tables 7 and 8 did not form suitable commercial-grade tablets. Tablet parameters are described in Example 4.

### Methods of Making Solid Cannabinoid Powders

Formulations K, L, M, and M[H]—Powders that formed suitable tablets

Formulations K, L, and M were prepared using the following process. First, CBD oil (containing 70% CBD w/w) were mixed with ethanol and heated in an oven at 60° C. The ethanol solubilizes the CBD oil and allows for improved absorption in the colloidal silicon dioxide. Next, the heated solution of CBD oil was added to colloidal silicon dioxide gradually. Next, the mannitol and sodium starch glycolate were added. Sodium stearyl fumarate was added last. The resulting powder was compressed in a tablet press.

Formulation M[H] was prepared using the following process. First, cannabinoid oil is heated in an oven to 60° C. Next, the heated non-crystalline cannabinoid oil was added to colloidal silicon dioxide. The composition containing colloidal silicon dioxide particles and non-crystalline cannabinoid oil was mixed using a high-shear mixer (i.e., a grinder). The composition was pulsed in the grinder for about 15 to about 30 times for one second per pulse.

Optionally, the composition was grinded continuously for an additional 20 seconds to homogenize the powder. Subsequently, the microcrystalline cellulose and croscarmellose sodium were added and mixed for 3 minutes in a bag, v-blender, or stand mixer. Lastly, the sodium stearyl fumarate was added and mixed for 1 minute. The short mixing time of 1 minute prevented over-blending of the sodium stearyl fumarate with the composition, which leads to worse dissolution.

Formulations L[H], K[H], J[H], I[H], H[H], G[H], A[H], B[H], C[H], D[H], E[H], and F[H]—Powders that Did not Form Suitable Commercial-Grade Tablets

Formulations L[H] and K[H] were prepared according to the following method. The cannabinoid oil was heated to 60° C. and combined with colloidal silicon dioxide particles and microcrystalline cellulose. The composition containing non-crystalline cannabinoid oil and silicon dioxide was grinded for a total of 80 seconds. The croscarmellose sodium was added and the composition was blended for three minutes. Sodium stearyl fumarate was added to the composition, and the composition was blended for one minute.

Formulations J[H], I[H], and H[H] were prepared according to the following method. The cannabinoid oil was heated to 60° C. and combined with microcrystalline cellulose. The composition containing non-crystalline cannabinoid oil and microcrystalline cellulose was grinded for a total of 80 seconds. The croscarmellose sodium was added and the composition was blended for two minutes. Sodium stearyl fumarate was added to the composition, and the composition was blended for one minute.

Formulation G[H] was prepared according to the following method. The cannabinoid oil was heated to 60° C. and combined with microcrystalline cellulose. The composition containing non-crystalline cannabinoid oil and microcrystalline cellulose was grinded for a total of 60 seconds. The croscarmellose sodium was added and the composition was blended for two minutes. Sodium stearyl fumarate was added to the composition, and the composition was blended for one minute.

Formulations A[H], B[H], C[H], D[H], and F[H] were produced according to the following process. First, powered CBD isolate, powdered CBG isolate, and cannabinoid oil were grinded with a mortar and pestle. Second, the composition containing cannabinoids was added to the colloidal silicon dioxide and grinded. Next, the composition containing cannabinoids and colloidal silicon dioxide was blended for five minutes with silicified microcrystalline cellulose, sodium starch glycolate, and optionally mannitol. Last, sodium stearyl fumarate was added to the composition, and the composition was blended for two minutes. These formulations did not show the improvement in dissolution. Without wishing to be bound by any one theory, the inventors hypothesize that powdered cannabinoids cannot effectively be introduced into colloidal silicon dioxide without first being heated to a liquid state.

Formulation E[F] was produced according to the following process. CBD isolate and colloidal silicon dioxide particles were grinded together with a mortar and pestle. The composition containing CBD isolate and colloidal silicon dioxide was blended with silicified microcrystalline cellulose and sodium starch glycolate. Last, sodium stearyl fumarate was added to the composition, and the composition was blended.

### Methods of Making Tablets

Each of the solid cannabinoid powders described above was compressed in a GlobePharma Mini Press-II tablet press. This press has ten stations of tooling with five stations of B tooling and five stations of D tooling. The tablet diameter of the tooling for each tablet is provided in Table 9.

TABLE 6

| | I | J | K | L | M | M[H] |
|---|---|---|---|---|---|---|
| | (% w/w) | (% w/w) | (% w/w) | (% w/w) | (% w/w) | (% w/w) |
| Non-crystalline | 41.9 | 22.1 | 22.1 | 22.1 | 22.1 | 19.4 |
| cannabinoid oil | | | | | | |
| Colloidal silicon | 41.9 | 58.2 | 58.2 | 29.1 | 29.1 | 31.1 |
| dioxide | | | | | | |
| Silicified | | | | | | 46.6 |
| Microcrystalline | | | | | | |
| cellulose | | | | | | |
| Mannitol | 12.3 | 14.3 | 14.3 | 40.7 | 43.4 | |
| Sodium starch | 2.0 | 2.7 | 2.7 | 5.5 | 2.7 | |
| glycolate | | | | | | |
| Croscarmellose | | | | | | 1.9 |
| sodium | | | | | | |
| Sodium stearyl | 2.0 | 2.7 | 2.7 | 2.7 | 2.7 | 1.0 |
| fumarate | | | | | | |

TABLE 7

| | L[H] | K[H] | J[H] | I[H] | H[H] | G[H] |
|---|---|---|---|---|---|---|
| | (% w/w) | (% w/w) | (% w/w) | (% w/w) | (% w/w) | (% w/w) |
| Non-crystalline | 19.4 | 19.4 | 19.4 | 16.2 | 19 | 19.4 |
| cannabinoid oil | | | | | | |
| Colloidal silicon | 31.1 | 15.5 | | | | |
| dioxide | | | | | | |
| Silicified | 46.6 | 62.1 | 77.7 | 81.0 | 76.2 | 77.7 |
| Microcrystalline | | | | | | |
| cellulose | | | | | | |
| Mannitol | | | | | | |
| Sodium starch | | | | | | |
| glycolate | | | | | | |
| Croscarmellose | 1.9 | 1.9 | 1.9 | 1.9 | 3.8 | 1.9 |
| sodium | | | | | | |
| Sodium stearyl | 1 | 1 | 1 | 0.8 | 1 | 1 |
| fumarate | | | | | | |

TABLE 8

| | F[H] | E[H] | D[H] | C[H] | B[H] | A[H] |
|---|---|---|---|---|---|---|
| | (% w/w) | (% w/w) | (% w/w) | (% w/w) | (% w/w) | (% w/w) |
| CBD Isolate | 25 | 10 | 6.7 | 25 | 25 | 26.5 |
| CBG Isolate | 9.8 | | 2.6 | 9.7 | 9.8 | 10.3 |
| Non-crystalline | 2.5 | | 0.7 | 2.5 | 2.5 | 2.7 |
| cannabinoid oil | | | | | | |
| Colloidal silicon | 37.5 | 17.5 | 50 | 37.5 | 37.5 | 37.1 |
| dioxide | | | | | | |
| Silicified | 21.5 | 68.8 | 37.3 | 22.5 | 22.5 | 18.6 |
| Microcrystalline | | | | | | |
| cellulose | | | | | | |
| Mannitol | | | | | | 1.9 |
| Sodium starch | 3 | 3 | 2 | 2 | 2 | 2 |
| glycolate | | | | | | |
| Croscarmellose | | | | | | |
| sodium | | | | | | |
| Sodium stearyl | 0.8 | 0.8 | 0.8 | 0.8 | 0.8 | 1 |
| fumarate | | | | | | |

TABLE 9

| | Tablet Diameter of Tooling | | Tablet Tooling Shape |
|---|---|---|---|
| I | 13 | mm | Round |
| J | 7 | mm | Round |
| K | 7 | mm | Round |
| L | 7 | mm | Round |
| M | 7 | mm | Round |
| M[H] | 0.25 | inches (in.) | Round, Flat Face Bevel Edge |
| L[H] | 0.25 | in. | Round, Flat Face Bevel Edge |
| K[H] | 0.25 | in. | Round, Flat Face Bevel Edge |
| J[H] | 0.25 | in. | Round, Flat Face Bevel Edge |
| I[H] | 0.25 | in. | Round, Flat Face Bevel Edge |
| H[H] | 0.25 | in. | Round, Flat Face Bevel Edge |
| G[H] | 0.25 | in. | Round, Flat Face Bevel Edge |
| D[H] | 0.3125 | in. | Round, Standard Convex |
| C[H] | 0.25 | in. | Round, Flat Face Bevel Edge |
| B[H] | 0.3125 | in. | Round, Standard Convex |
| A[H] | 0.3125 | in. | Round, Standard Convex |

### Example 4—Tablet Parameters

Having prepared various tablets containing cannabinoid oil (see Example 3), the tablets were evaluated for their suitability as pharmaceutical products.

Methods for determining hardness or compression force of tablet

The hardness was determined using a “breaking force” test. Briefly, tablets were placed in a SOTAX HT1 Tablet Hardness Tester (FIG. 4). A force was applied to the tablet to cause fracture. The force at which the tablet fractures is the tablet's hardness.

The compression force of the tablet was determined by measuring the force acting on the tablet when the tablet was pressed in the tablet press.

Methods for determining disintegration of tablet

Each tablet was placed into a receptacle containing 10 milliliters (mL) of 2% (w/v) sodium lauryl sulfate (SLS). The amount of time that the tablet took to disintegrate was recorded. The tablet was determined to be disintegrated when no palpably firm core was remaining in the SLS.

Methods for determining dissolution of tablet

Tablets were analyzed for their dissolution performance using a Sotax 6-vessel Dissolution Bath (a USP Type II Dissolution Apparatus) in tandem with the Pion μDiss Profiler, which is a fiber optic dissolution unit. Each vessel is filled with 900 milliliters (mL) of 1% w/v Tween 80 (Polysorbate 80) solution. The vessel is heated to 37° C., and the stir speed is set to 100 revolutions per minute (RPM). Paddles were used for stirring. Each vessel had a single Pion probe submerged in the media to read the absorbance, which was translated to Percent (%) Dissolved.

Table 10 summarizes the parameters of each tablet. The data showed that tablets comprising formulations D[H], C[H], B[H], and A[H] were not hard enough. The hardness of these tablets was less than 50 Newtons (N). Tablets comprising formulations G[H], H[H], I[H], J[H], K[H], and L[H] each had suitable hardness, but the dissolution was not suitable (dissolution in 60 minutes<65%). Tablets comprising formulations M[H], M, L, K, J, and I each resulted in tablets of suitable hardness (50-100 N) and disintegration (1-10 minutes in 2% SLS).

This data showed that the excipient combination affected the suitability of the tablets for pharmaceutical formulation. This data also showed that the method of preparing the solid cannabinoid powder affects the suitability of a tablet as a pharmaceutical. Specifically, initial high shear mixing of non-crystalline cannabinoid oil and the colloidal silicon dioxide particles in the absence of other excipients is critical. The powder of tablet M[H] is prepared using this critical initial step and has the appropriate hardness and dissolution for a pharmaceutical. In contrast, the powder of tablet L[H] is produced by initial high shear mixing of colloidal silicon dioxide particles and non-crystalline cannabinoid oil with another excipient (i.e., silicified microcrystalline cellulose). In contrast to tablet M[H], Tablet L[H] was not a suitable pharmaceutical because it did not have the appropriate dissolution. The dissolution of Tablet M[H] was 77.5%, whereas the dissolution of Tablet L[H] was 57.3%.

TABLE 10
Tablet Parameters

| | | | Average | |
|---|---|---|---|---|
| | | Hardness | Compression | |
| | Disintegration | (Newtons (N)) | Force | Dissolution |
| I | N.D. | N.D. | 10 | N.D. |
| J | 1 minute in 2% | N.D. | 12.7 | N.D. |
| | sodium lauryl | | | |
| | sulfate (SLS) | | | |
| K | 1 minute in 2% | N.D. | 10 | N.D. |
| | SLS | | | |
| L | 1 minute in 2% | N.D. | N.D. | N.D. |
| | SLS | | | |
| M | 1 minute in 2% | N.D. | N.D. | N.D. |
| | SLS | | | |
| M[H] | N.D. | 73 | N.D. | 77.5% |
| L[H] | N.D. | 83 | N.D. | 57.3% |
| K[H] | N.D. | 70 | N.D. | 56.0% |
| J[H] | N.D. | 90 | N.D. | 61.4% |
| I[H] | N.D. | 90 | N.D. | 58.4% |
| H[H] | N.D. | | N.D. | 64.9% |
| G[H] | N.D. | | N.D. | 60.1% |
| F[H] | N.D. | N.D. | N.D. | N.D. |
| E[H]* | N.D. | | N.D. | 63.4% |
| D[H] | N.D. | <50 N | N.D. | 75.2% |
| C[H] | N.D. | <50 N | N.D. | 63.4% |
| B[H] | N.D. | 17 | N.D. | 49.7% |
| A[H] | N.D. | 10-20 | N.D. | 33.7% (30 min) |

*Powder blend

### Example 5—Capsules Containing Non-Crystalline Cannabinoid Oil

Examples 1 and 2 provide one example of the stabilization and dissolution benefits of colloidal silicon dioxide and cannabinoid combination for capsules. This example describes the development and formulation of additional capsules.

Specifically, it was discovered that additional ingredients could improve the commercial properties of cannabinoid-based capsules. Assessment of each of the tested formulations is described in more detail below and in Example 6.

Capsules comprising combinations of a non-crystalline cannabinoid oil, colloidal silicon dioxide, a filler (i.e., microcrystalline cellulose), a disintegrant (i.e. sodium starch glycolate), a lubricant (i.e. sodium stearyl fumarate), and flavorings (i.e., myrcene or linalool) were prepared using the methods described below. The components of each composition evaluated are contained in Tables 11 and 12. Each table contains the percent by weight of each component of the compositions.

### Methods of Making Solid Cannabinoid Powders

Formulations G[C], F[C], E[C], and D[C]-Powders that Formed Suitable Capsules

Formulation D[C] was generated according to the following process. The non-crystalline cannabinoid oil was added to colloidal silicon dioxide particles and combined vigorously with a mortar and pestle. The composition containing colloidal silicon dioxide particles and non-crystalline cannabinoid oil was transferred to a stainless steel mixing bowl. The cannabinoid isolates, silicified microcrystalline cellulose, and lubricant were added to the bowl, and mixed using a stand mixer at the lowest speed setting for five minutes. 69 grams of the formulation was used to fill 300 size 0 capsule shells.

Formulation E-C was generated according to the following process. The non-crystalline cannabinoid oil and terpenes were combined with colloidal silicon dioxide particles and grinded and pulsed until uniform. The mixture of colloidal silicon dioxide particles and non-crystalline cannabinoid oil were transferred to a stainless steel mixing bowl. CBD and CBG isolate was added to the bowl, and the mixture was mixed at the lowest speed for five minutes with a flat beater attachment. Sodium stearyl fumarate was added to the bowl, and the composition was mixed for 1 minute. The composition was used to fill size 2 capsule shells.

Formulations G-C and F-C were generated according to the following process. The non-crystalline cannabinoid oil and terpenes were combined with colloidal silicon dioxide and grinded and pulsed ten times. The mixture of colloidal silicon dioxide and non-crystalline cannabinoid oil were transferred to a stainless steel mixing bowl. CBD and CBG isolate was added to the bowl, and the mixture was mixed at the lowest speed for ten minutes with a flat beater attachment. Sodium stearyl fumarate was added to the bowl, and the composition was mixed for 2 minute. The composition was used to fill size 2 capsule shells.

Formulations C[C], B[C], and A[C]-Powders that Did not Form Suitable Commercial-Grade Capsules

Formulations C[C], B[C], and A[C] were generated according to the following process. CBD, CBG, and non-crystalline cannabinoid oil were combined with colloidal silicon dioxide particles and grinded together in a mortar and pestle. Silicified microcrystalline cellulose, sodium starch glycolate were added to the mixture, and it was blended. Sodium stearyl fumarate was added last, and the mixture was blended.

### Capsule Preparation

The solid cannabinoid powders above were filled in gelatin or vegetarian shells. A Torpac ProFiller® 3700 capsule filler was used to fill the capsule shells with Cannabinoid Products. 230 mg of the D[C] Cannabinoid Product was added to size 0 gelatin capsules. 123.8 mg of the G[C], F[C], or E[C] Cannabinoid Products were added to size 2 gelatin capsules. FIG. 2 shows an image of the Torpac ProFiller® 3700 capsule filler. The vegetarian shells contained hydroxypropylmethylcellulose.

TABLE 11
Capsule Parameters

| | G[C] (% | F[C] (% | E[C] (% | D[C] (% |
|---|---|---|---|---|
| | w/w) | w/w) | w/w) | w/w) |
| Cannabidiol (CBD) | 40.4 | 40.4 | 40.4 | 21.7 |
| Isolate | | | | |
| Cannabigerol (CBG) | 11.7 | 11.7 | 11.7 | 8.5 |
| Isolate | | | | |
| Non-crystalline | | | | 2.2 |
| cannabinoid oil | | | | |
| (broad spectrum) | | | | |
| Non-crystalline | 8.1 | 8.1 | 8.1 | |
| cannabinoid oil (D8) | | | | |
| Colloidal silicon | 39 | 39 | 39 | 32.4 |
| dioxide | | | | |
| Silicified | | | | 34.8 |
| Microcrystalline | | | | |
| cellulose | | | | |
| Sodium stearyl | 0.4 | 0.4 | 0.4 | 0.4 |
| fumarate | | | | |
| Myrcene | 0.2 | 0.2 | 0.2 | |
| Linalool | 0.2 | 0.2 | 0.2 | |

TABLE 12
Capsule Parameters

| | C[C] | B[C] | A[C] |
|---|---|---|---|
| | (% w/w) | (% w/w) | (% w/w) |
| Cannabidiol (CBD) Isolate | 6.7 | 6.7 | 6.7 |
| (not loaded) | | | |
| Cannabigerol (CBG) | 2.6 | 2.6 | 2.6 |
| Isolate (not loaded) | | | |
| Non-crystalline | 0.7 | 0.7 | 0.7 |
| cannabinoid oil (broad | | | |
| spectrum) | | | |
| Non-crystalline | | | |
| cannabinoid oil (D8) | | | |
| Colloidal silicon dioxide | 17.5 | 15 | 12.5 |
| Silicified Microcrystalline | 68.8 | 71.3 | 73.8 |
| cellulose | | | |
| Sodium starch glycolate | 3 | 3 | 3 |
| Sodium stearyl fumarate | 0.8 | 0.8 | 0.8 |

### Example 6—Capsule Parameters

Having prepared various capsules containing non-crystalline cannabinoid oil (see Example 5), the capsules were evaluated for their suitability as pharmaceutical products.

### Methods for Determining Dissolution of Capsule

Dissolution measurements were performed in a Sotax 6-vessel Dissolution Bath (a USP Type 1 Dissolution Apparatus) in tandem with the Pion μDiss Profiler, which is a fiber optic dissolution unit. Each vessel was filled with 900 milliliters (mL) of i) fasted state simulated intestinal fluid (FaSSIF), or ii) a fed state simulated intestinal fluid (FeSSIF). The FaSSIF contained about 3 mM taurocholate, 0.75 mM phospholipids, 148 mM sodium ions, 106 mM chloride, and 29 mM phosphate ions, thereby modeling the conditions present in fasted small intestine gastrointestinal fluid. The pH of FaSSIF was about 6.5. FeSSIF contained about 15 mM taurocholate, 3.75 mM phospholipids, 319 mM sodium ions, 203 mM chloride, and 144 mM acetic acid. The pH of FeSSIF was about 5.0. FeSSIF contained similar osmolality and pH to fed intestinal fluid. A iii) control polysorbate 80 (TWEEN®80) solution was also tested in the experiment.

The vessel was heated to 37° C., and the stir speed was set to 100 revolutions per minute (RPM). Each vessel had a single Pion probe submerged in the media to read the absorbance, which was translated to Percent (%) Dissolved. Baskets were used for stirring the capsules.

Table 13 summarizes the results from this experiment as percent dissolution within 60 minutes (higher value indicates greater dissolution).

### Results

Table 13 provides the dissolution data for each capsule.

TABLE 13

| Formulations | Control | FaSSIF | FeSSIF |
|---|---|---|---|
| G[C] | N.D. | N.D. | N.D. |
| F[C] | 27.9% | 12.0% | 37.2% |
| E[C] | 45.4% | 8.4% | 38.6% |
| D[C] | 37.1% | 18.9% | 41.7% |
| C[C]* | 83.6% | 27.7% | N.D. |
| B[C]* | 60.1% | 23.7% | N.D. |
| A[C]* | 70.0% | 25.8% | N.D. |

*Powder blend, not filled into capsule

### Example 7—Evaluation of Heating Cannabinoid Oil Prior to Incorporation into Colloidal Silicon Dioxide

Earlier examples describe various techniques for producing proprietary tablets and capsules containing non-crystalline cannabinoid oil loaded into colloidal dioxide parties. This example evaluates the effect of heating cannabinoid oil prior to incorporation with colloidal silicon dioxide.

A control formulation without heating cannabinoid oil was prepared using the following process and the formulation is shown below in Table 14. The semi-liquid cannabinoid oil was weighed into a high-shear mixer (i.e. a grinder) and was pulsed 10 times for one second per pulse and then run continuously for 10 seconds. Colloidal silicon dioxide particles (Aeroperl 300) were added to the high-shear mixer and pulsed 15 times for one second per pulse.

The silicified microcrystalline cellulose (ProSolv 730) and croscarmellose sodium were added to the existing blend and mixed for 3 minutes in a bag (a v-blender or stand mixer could be used). Lastly, the sodium stearyl fumarate was added and mixed for 1 minute. The short mixing time of 1 minute prevented over-blending of the sodium stearyl fumarate. The tablet blend was then compressed to at target weight of 103 mg and a target hardness of approximately 70 Newtons (N). The tablet blend was also filled into Size 0 gelatin capsule shells at the same proportions to observe the dissolution results without tablet disintegration.

Test cannabinoid compositions with heated cannabinoid oil were produced as above, with the exception that in the test compositions, the cannabinoid oil was first heated to 60° C. before mixing with the colloidal silicon dioxide particles. Ingredient breakdowns for the Control and Test compositions are provided in Table 14, below.

TABLE 14
20 mg Control and Test Tablet and Capsule Formulation

| | | mg/tablet or |
|---|---|---|
| Component | % w/w | capsule |
| Cannabinoid oil (heated-non-crystalline | 19.40% | 20.0 |
| vs. semi-solid, room temperature) | | |
| Silicified microcrystalline cellulose | 46.60% | 48.0 |
| (ProSolv 730, filler) | | |
| Colloidal silica (Aeroperl 300 Pharma) | 31.10% | 32.0 |
| Croscarmellose sodium (disintegrant) | 1.90% | 2.0 |
| Sodium Stearyl Fumarate (lubricant) | 1.00% | 1.0 |
| Total | 100.00% | 103.0 |

The capsules and tablets made following the process above were tested for dissolution in fasted state simulated intestinal fluid (FaSSIF) and fed state simulated intestinal fluid (FeSSIF) as described in Example 2, above, using Type I/II Dissolution apparatus (Basket and Paddle attachments, respectively).

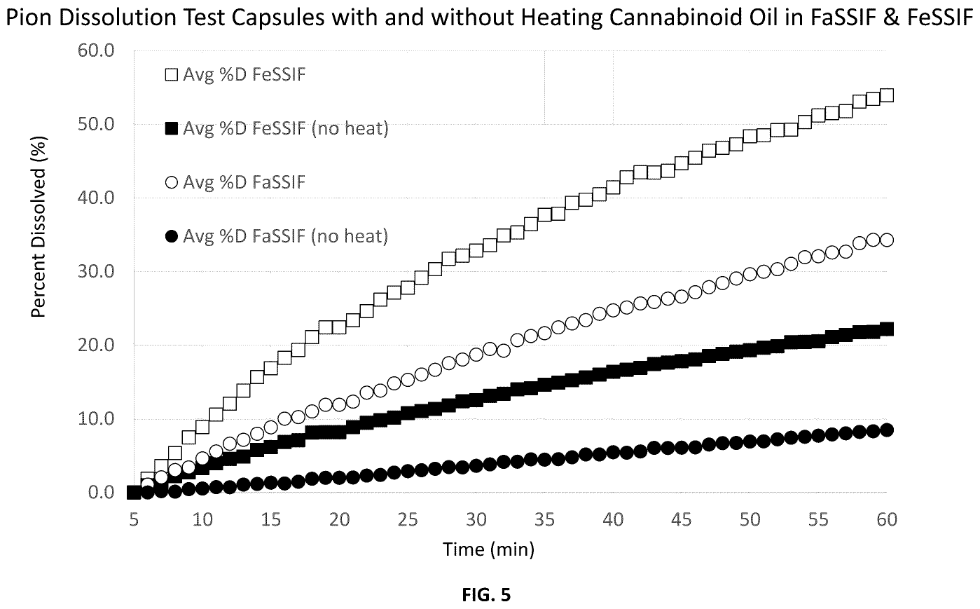

These results show that heat-loaded cannabinoid oil is more readily available to patients post ingestion. Specifically, Test formulation made by heating the cannabinoid oil prior to loading, exhibits significantly higher percent dissolved at 60 minutes compared to control formulations in both simulated Fasted and Fed media as shown below in FIG. 5. Results also summarized in Table 15, below. Similar results were seen when the Test and Control formulations were dissolved in 1% Tween 80. The data was normalized to 5 minutes after contacting dissolution media to account for any variation in capsule or tablet dissolving. The R² values of the slopes from a linear regression best fit line for each data series in FIG. 5 are shown in Table 16.

TABLE 15
Dissolution Comparison of Formulations made with and without heating
cannabinoid oil prior to high-shear mixing with Colloidal silica

| | Dissolution in | Dissolution in |
|---|---|---|
| | Fasted (FaSSIF) | Fed (FeSSIF) |
| | pH 6.50 | pH 5.0 |
| 20 mg Capsules - No Heating | 21.8% | 52.9% |
| Formulation | | |
| Dissolution Bath Type I | | |
| Apparatus - Baskets | | |
| 20 mg Tablets - No Heating | 13.8% | 45.9% |
| Formulation | | |
| Dissolution Bath Type II | | |
| Apparatus - Paddles | | |
| 20 mg Tablets - Standard Heating | 31.1% | 69.7% |
| Formulation | | |
| Dissolution Batch Type II | | |
| Apparatus - Paddles | | |
| % Improvement from Heating vs No | +17.3% | +23.8% |
| Heating of Cannabinoid Oil (For | | |
| Tablets) | | |

TABLE 16
R² Values of the linear regression best fit
lines for each data series shown in FIG. 5

| Condition | R² |
|---|---|
| Avg % D FeSSIF | 0.9703 |
| Avg % D FeSSIF (no heat) | 0.9846 |
| Avg % D FaSSIF | 0.9914 |
| Avg % D FaSSIF (no heat) | 0.9981 |

As noted above, and in FIG. 5, test formulations in which the cannabinoid oil is heated before loading unexpectedly exhibit higher dissolution rates in both FeSSIF media and FaSSIF media. Moreover, all conditions exhibited substantially linear dissolutions (See Table 9). Note that this evaluation was conducted at relatively low cannabinoid oil to colloidal silicon dioxide tablet ratios.

### Example 8. Varying the Non-Crystalline Cannabinoid Oil Loading to Colloidal Silicon Dioxide Particles Ratio

This study evaluated cannabinoid compositions with various cannabinoid: colloidal silicon dioxide particle ratios to determine the amount of cannabinoid dose that can be delivered via capsule/tablet under current FDA guidelines.

As noted in earlier parts of this document, formulations of the present disclosure do not require dilution of cannabinoids prior to loading onto colloidal silicon dioxide particles. This was hypothesized to permit for higher cannabinoid loading per unit dose, due to the higher cannabinoid content of the undiluted non-crystalline cannabinoid oil, and the higher non-crystalline cannabinoid oil to colloidal silicon dioxide particles ratios achievable through loading of heated non-crystalline cannabinoid oil.

Higher non-crystalline cannabinoid oil to colloidal silicon dioxide particle ratios are particularly desirable, as it reduces the amount of colloidal silicon dioxide administered to patients per unit dose of cannabinoid. See the FDA's Inactive Ingredient Database, for limits on oral administration of Silicon Dioxide. (database available at world wide web fda.gov/drugs/drug-approvals-and-databases/inactive-ingredients-database-download, and is incorporated herein in its entirety for all purposes). Thus, formulations of the present disclosure were hypothesized to be able to deliver higher cannabinoid dosages without exceeding FDA limits on consumption of silicon dioxide.

Test formulations exhibiting non-crystalline cannabinoid oil: colloidal silicon dioxide particle ratios from 0.5:1 to 1.5:1 were produced according to the methods of Example 1. Recipes for formulations of this example are shown below in Table 17. All formulations were capped at a 10 mg cannabinoid dose, while varying the amount of colloidal silicon dioxide particles. These formulations were then hand-filled into Size 0 gelatin capsules.

TABLE 17
Varying Colloidal Silicon Dioxide Particle Loading
with Non-Crystalline Cannabinoid Oil

| | | |
|---|---|---|
| Component | % w/w | mg/capsule |

1.5:1 Non-crystalline cannabinoid oil:Colloidal Silicon Dioxide Particle

| | | |
|---|---|---|
| Non-crystalline | 60% | 10 |
| cannabinoid oil | | |
| colloidal silicon | 40% | 6.667 |
| dioxide particles | | |
| Total | 100% | 16.667 |

1.25:1 Oil:Colloidal Silicon Dioxide Particle

| | | |
|---|---|---|
| Non-crystalline | 56% | 10 |
| cannabinoid oil | | |
| colloidal silicon | 44% | 8 |
| dioxide particles | | |
| Total | 100% | 18 |

1:1 Oil:Colloidal Silicon Dioxide Particle

| | | |
|---|---|---|
| Non-crystalline | 50% | 10 |
| cannabinoid oil | | |
| colloidal silicon | 50% | 10 |
| dioxide particles | | |
| Total | 100% | 20 |

0.75:1 Oil:Colloidal Silicon Dioxide Particle

| | | |
|---|---|---|
| Non-crystalline | 42.86% | 10 |
| cannabinoid oil | | |
| colloidal silicon | 57.14% | 13.33 |
| dioxide particles | | |
| Total | 100% | 23.33 |

0.5:1 Oil:Colloidal Silicon Dioxide Particle

| | | |
|---|---|---|
| Non-crystalline | 33.33% | 10 |
| cannabinoid oil | | |
| colloidal silicon | 66.66% | 20 |
| dioxide particles | | |
| Total | 100% | 30 |

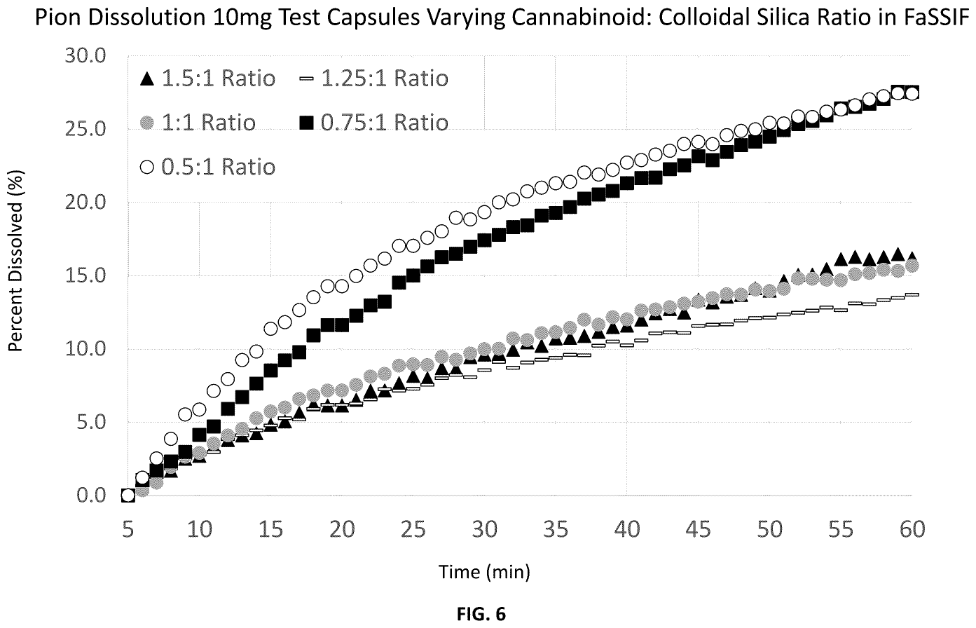

TABLE 18
R² Values of the linear regression best fit
lines for each data series shown in FIG. 6

| Ratio | R² |
|---|---|
| 1.5:1 | 0.9863 |
| 1.25:1 | 0.9645 |
| 1:1 | 0.9521 |
| 0.75:1 | 0.9629 |
| 0.5:1 | 0.9198 |

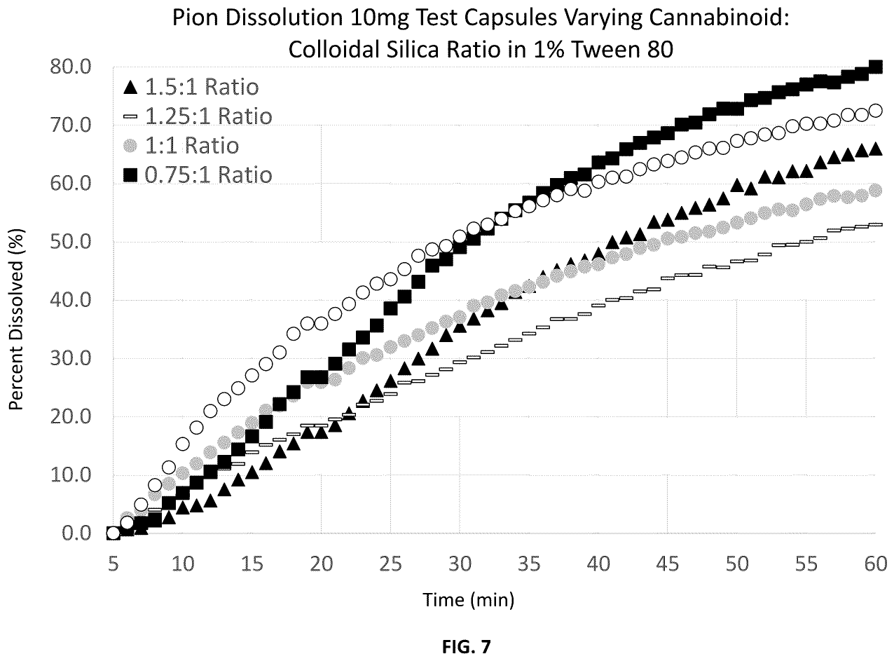

TABLE 19
R² Values of the linear regression best fit
lines for each data series shown in FIG. 7

| Ratio | R² |
|---|---|
| 1.5:1 | 0.9869 |
| 1.25:1 | 0.9886 |
| 1:1 | 0.9625 |
| 0.75:1 | 0.9667 |
| 0.5:1 | 0.9263 |

The capsules that were prepared by following the Formulations in Table 17 were tested for dissolution in both FaSSIF (Fasted Simulated State Intestinal Fluid) and 1% Tween 80. The dissolution results are found in FIG. 6 (FaSSIF Dissolution) and FIG. 7 (1% Tween 80 Dissolution). The data was normalized to 5 minutes after contacting dissolution media to account for any variation in capsule or tablet dissolving.

This study demonstrated that it was possible to load non-crystalline cannabinoid oil into colloidal silicon dioxide particles at ratios that were more than three times higher than what had been previously reported in the art. All tested ratios exhibited substantially linear dissolutions (See Tables 18 and 19). Interestingly and surprisingly, formulations with higher cannabinoid to colloidal silicon dioxide particles ratios appeared to exhibit slower release profiles and higher R² values that are potentially valuable for cannabinoid treatments requiring persistent drug delivery without dissolution spikes (e.g., chronic pain management through THC).

### Example 9. Comparison to Prior Generation Formulations

This experiment is to determine how the methods and formulations of the current disclosure compare to prior art formulations using cannabinoids dissolved in solvents and loaded onto colloidal silicon dioxide as disclosed in WO2021151169A1.

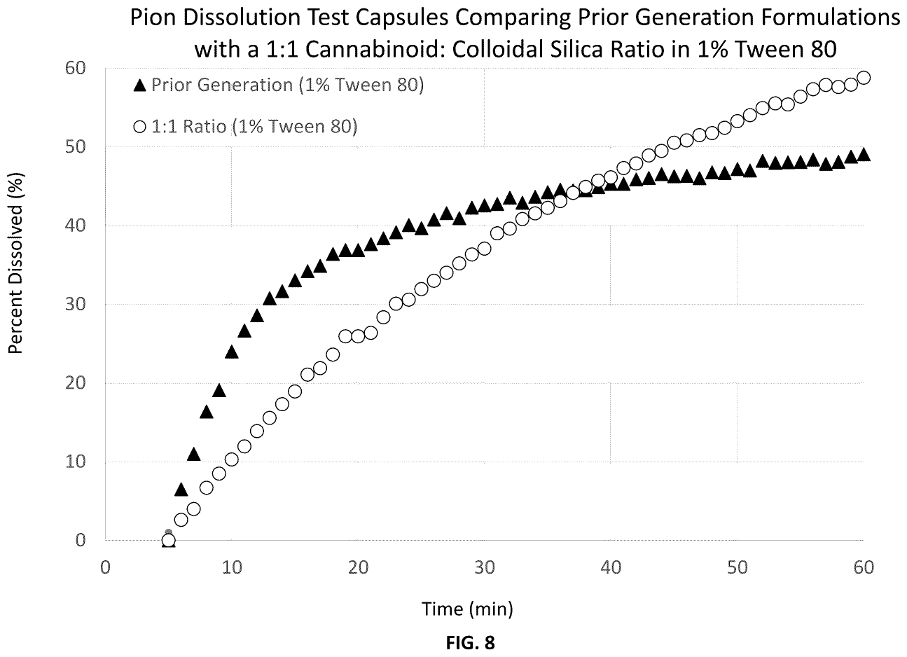

The capsules were prepared by following the Formulations in Table 20 and were tested for dissolution 1% Tween 80, which was used in place of FeSSIF (Fed Simulated State Intestinal Fluid). The dissolution results are shown in FIG. 8. The data was normalized to 5 minutes after contacting dissolution media to account for any variation in capsule or tablet dissolving. The R² values of the linear regression best fit line for each data series in FIG. 8 are shown in Table 21. The data was normalized to 5 minutes after contacting dissolution media to account for any variation in capsule or tablet dissolving.

TABLE 20
Comparison of 10 mg Cannabinoid Oil from Prior
Generation Formulation (WO2021151169A1) to 1:1
Non-Crystalline Cannabinoid oil:Colloidal Silicon
Dioxide Particle of the Present Disclosure

| | | | Cannabinoid Product | |
|---|---|---|---|---|
| | | | 1:1 Oil:Colloidal | |
| | Prior Generation | | Silicon Dioxide | |
| | (WO2021151169A1) | | Particle | |
| | | mg/ | | mg/ |
| Component | % w/w | capsule | % w/w | capsule |
| Cannabinoid oil | 3.70% | 10 | 50 | 10 |
| (unheated in Pior | | | | |
| Generation) | | | | |
| MCT Oil | 18.52% | 50 | NA | NA |
| Colloidal silica | 22.23% | 60 | 50 | 10 |
| Castor Oil | 4.44% | 12 | NA | NA |
| Microcrystalline | 34.81% | 94 | NA | NA |
| cellulose (Vivapur 102) | | | | |
| Tricalcium phosphate | 15.56% | 42 | NA | NA |
| Sodium Stearyl Fumarate | 0.74% | 2 | NA | NA |
| Total | 100.00% | 270 | 100% | 20 |

TABLE 21
R² Values of the linear regression best fit
lines for each data series shown in FIG. 8

| Condition | R² |
|---|---|
| Prior Art | 0.7127 |
| CP 1:1 | 0.9625 |

### Results

FIG. 8 shows rapid dissolution followed by periods of slower dissolution of the prior generation formulation, resulting in a non-linear dissolution (See Table 21). The results of this experiment demonstrated that prior generation compositions are diluted in additional liquid and/or lipid components exhibited a non-linear dissolution rate.

### NUMBERED EMBODIMENTS

Embodiment 1. A solid Cannabinoid Product comprising:
(a) non-crystalline cannabinoid oil; and
(b) colloidal silicon dioxide particles comprising internal storage cavities, wherein the non-crystalline cannabinoid oil is loaded into the colloidal silicon dioxide storage particles, and wherein the loaded non-crystalline cannabinoid oil to colloidal silicon dioxide particle ratio is about 0.5:1 to 3:1 by weight.

Embodiment 2. The solid Cannabinoid Product of embodiment 1, wherein the non-crystalline cannabinoid oil comprises at least 80%, at least 90%, at least 95%, at least 99% cannabinoid by weight.

Embodiment 3. The solid Cannabinoid Product of any one of embodiments 1-2, wherein the non-crystalline cannabinoid oil is not diluted in a non-cannabis solvent.

Embodiment 4 The solid Cannabinoid Product of embodiment 3, wherein the non-cannabis solvent is an alcohol.

Embodiment 5. The solid Cannabinoid Product of embodiment 3, wherein the non-cannabis solvent is medium-chain triglycerides.

Embodiment 6. The solid Cannabinoid Product of embodiment 3, wherein the non-cannabis solvent is castor oil.

Embodiment 7. The solid Cannabinoid Product of embodiment 3, wherein the non-cannabis solvent is Kolliphor EL.

Embodiment 8. The solid Cannabinoid Product of any one of embodiments 1-7, wherein the loaded non-crystalline cannabinoid oil to colloidal silicon dioxide particle ratio is about 1:1 to 2:1 by weight.

Embodiment 9. The solid Cannabinoid Product of any one of embodiments 1-8, wherein the loaded non-crystalline cannabinoid oil to colloidal silicon dioxide particle ratio is about 1:5 by weight.

Embodiment 10. The solid Cannabinoid Product of any one of embodiment 1-9, comprising total non-cannabinoid solid ingredients; wherein said total non-cannabinoid solid ingredients are less than about 95%, about 90%, about 85%, about 80%, about 75%, about 70%, about 65%, about 60%, about 55%, about 50%, about 45%, about 40%, about 35%, about 30%, about 25%, or about 20% of the Cannabinoid Product by weight.

Embodiment 11. The solid Cannabinoid Product of any one of embodiments 1-10, wherein the non-crystalline cannabinoid oil is loaded onto the colloidal silicon dioxide particles in the absence of any other solid ingredients.

Embodiment 12. The solid Cannabinoid Product of any one of embodiments 1-11, wherein the Cannabinoid Product comprises less than 10%, 9%, 8%, 7%, 6%, 5%, 4%, 3%, 2%, 1% w/w cyclodextrin.

Embodiment 13. The solid Cannabinoid Product of any one of embodiments 1-11, wherein the Cannabinoid Product does not comprise cyclodextrin.

Embodiment 14. The solid Cannabinoid Product of any one of embodiments 1-13, wherein the Cannabinoid Product comprises less than 10%, 9%, 8%, 7%, 6%, 5%, 4%, 3%, 2%, 1% w/w emulsifier.

Embodiment 15. The solid Cannabinoid Product of any one of embodiments 1-14, wherein the Cannabinoid Product does not comprise an emulsifier.

Embodiment 16. The solid Cannabinoid Product of any one of embodiments 1-15, wherein the solid Cannabinoid Product has a hardness of from 50 Newtons (N) to 100 N.

Embodiment 16.1 The solid Cannabinoid Product of any one of embodiments 1-16, wherein the Cannabinoid Product exhibits a linear dissolution rate as measured between 5-60 minutes in 1% Tween 80 media, Fasted State Simulated Intestinal Fluid (FaSSIF) media, and/or Fed State Simulated Intestinal Fluid (FeSSIF) media.

Embodiment 16.2 The solid Cannabinoid Product of any one of embodiments 1-16.1, wherein at least 70% of the non-crystalline cannabinoid oil is loaded into the colloidal silicon dioxide storage particles.

Embodiment 17. A solid Cannabinoid Product comprising:
(a) non-crystalline cannabinoid oil; and
(b) colloidal silicon dioxide particles comprising internal storage cavities,
   wherein at least 90% of the non-crystalline cannabinoid oil is loaded into the colloidal silicon dioxide storage particles, and wherein the non-crystalline cannabinoid oil content is at least 30% w/w of the colloidal silicon dioxide particle content.

Embodiment 18. The solid Cannabinoid Product of embodiment 17, wherein the non-crystalline cannabinoid oil is not diluted in a non-cannabis solvent.

Embodiment 19. The solid Cannabinoid Product of any one of embodiments 17-18, wherein the non-cannabis solvent is an alcohol.

Embodiment 20. The solid Cannabinoid Product of any one of embodiments 17-18, wherein the non-cannabis solvent is medium-chain triglycerides.

Embodiment 21. The solid Cannabinoid Product of any one of embodiments 17-18, wherein the non-cannabis solvent is castor oil.

Embodiment 22. The solid Cannabinoid Product of any one of embodiments 17-18, wherein the non-cannabis solvent is Kolliphor EL.

Embodiment 23. The solid Cannabinoid Product of any one of embodiments 17-22, wherein the non-crystalline cannabinoid oil comprises at least 50% w/w of the colloidal silicon dioxide particles comprising internal storage cavities.

Embodiment 24. The solid Cannabinoid Product of any one of embodiments 17-23, wherein the non-crystalline cannabinoid oil comprises at least 70% w/w of the colloidal silicon dioxide particles comprising internal storage cavities.

Embodiment 25. The solid Cannabinoid Product of any one of embodiments 17-24, comprising total non-cannabinoid solid ingredients; wherein said total non-cannabinoid solid ingredients are less than about 95%, about 90%, about 85%, about 80%, about 75%, about 70%, about 65%, about 60%, about 55%, about 50%, about 45%, about 40%, about 35%, about 30%, about 25%, or about 20% of the Cannabinoid Product by weight.

Embodiment 26. The solid Cannabinoid Product of any one of embodiments 17-25, wherein the non-crystalline cannabinoid oil is loaded onto the colloidal silicon dioxide particles in the absence of any other solid ingredients.

Embodiment 27. The solid Cannabinoid Product of any one of embodiments 17-26, wherein the Cannabinoid Product comprises less than 10%, 9%, 8%, 7%, 6%, 5%, 4%, 3%, 2%, 1% w/w cyclodextrin.

Embodiment 28. The solid Cannabinoid Product of any one of embodiments 17-27, wherein the Cannabinoid Product does not comprise cyclodextrin.

Embodiment 29. The solid Cannabinoid Product of any one of embodiments 17-28, wherein the Cannabinoid Product comprises less than 10%, 9%, 8%, 7%, 6%, 5%, 4%, 3%, 2%, 1% w/w emulsifier.

Embodiment 30. The solid Cannabinoid Product of any one of embodiments 17-29, wherein the Cannabinoid Product does not comprise an emulsifier.

Embodiment 31. The solid Cannabinoid Product of any one of claims 17-30, wherein the solid Cannabinoid Product has a hardness of from 50 Newtons (N) to 100 N.

Embodiment 31.1 The solid Cannabinoid Product of any one of embodiments 17-31, wherein the Cannabinoid Product exhibits a linear dissolution rate as measured between 5-60 minutes in 1% Tween 80 media, Fasted State Simulated Intestinal Fluid (FaSSIF) media, and/or Fed State Simulated Intestinal Fluid (FeSSIF) media.

Embodiment 31.2 The solid Cannabinoid Product of any one of embodiments 17-31.1, wherein at least 70% of the non-crystalline cannabinoid oil is loaded into the colloidal silicon dioxide storage particles.

Embodiment 32. A cannabinoid daily patient dose comprising
(a) non-crystalline cannabinoid oil comprising a cannabinoid; and
(b) colloidal silicon dioxide particles comprising internal storage cavities
   wherein at least 90% of the non-crystalline cannabinoid oil is loaded into the colloidal silicon dioxide particles comprising internal storage cavities, and wherein said daily dose comprises greater than 50 mg of the cannabinoid.

Embodiment 33. The cannabinoid daily dose of embodiment 32, wherein the daily dose is a pill.

Embodiment 34. The cannabinoid daily dose of any one of embodiments 32-33, wherein the daily dose is a tablet.

Embodiment 35. The cannabinoid daily dose of any one of embodiments 32-33, wherein the daily dose is a single pill.

Embodiment 36. The cannabinoid daily dose of any one of embodiments 32-35, wherein the single pill comprises about 75 mg to about 150 mg cannabinoid.

Embodiment 37. The cannabinoid daily dose of any one of embodiments 32-36, wherein the daily dose is at least two pills.

Embodiment 38. The cannabinoid daily dose of any one of embodiments 32-37, wherein the daily dose is a single tablet.

Embodiment 39. The cannabinoid daily dose of any one of embodiments 32-38, wherein the single tablet comprises about 75 mg to about 150 mg cannabinoid.

Embodiment 40. The cannabinoid daily dose of any one of embodiments 32-39, wherein the daily dose is at least two tablets.

Embodiment 41. The cannabinoid daily dose of any one of embodiments 32-40, wherein the daily dose comprises about 0.1 mg/kg/day to about 25 mg/kg/day of cannabinoid.

Embodiment 42. The cannabinoid daily dose of any one of embodiments 32-41, wherein the daily dose comprises about 1 mg/kg/day of non-crystalline cannabinoid oil.

Embodiment 43. The cannabinoid daily dose of any one of embodiments 32-42, wherein the single pill comprises about 50 mg to about 100 mg colloidal silicon dioxide particles comprising internal storage cavities.

Embodiment 44. The cannabinoid daily dose of any one of embodiments 32-43, wherein the single tablet comprises about 50 mg to about 100 mg colloidal silicon dioxide particles comprising internal storage cavities.

Embodiment 45. The cannabinoid daily dose of any one of embodiments 32-44, wherein the non-crystalline cannabinoid oil in the Cannabinoid Product is in solid form.

Embodiment 46. The cannabinoid daily dose of any one of embodiments 32-45, wherein the non-crystalline cannabinoid oil is not diluted in a non-cannabis solvent.

Embodiment 47. The cannabinoid daily dose of any one of embodiments 32-46, wherein the non-cannabis solvent is an alcohol.

Embodiment 48. The cannabinoid daily dose of any one of embodiments 32-47, wherein the non-cannabis solvent is medium-chain triglycerides.

Embodiment 49. The cannabinoid daily dose of any one of embodiments 32-48, wherein the non-cannabis solvent is castor oil.

Embodiment 50. The cannabinoid daily dose of any one of embodiments 32-49, wherein the non-cannabis solvent is Kolliphor EL.

Embodiment 51. The cannabinoid daily dose of any one of embodiments 32-50, comprising total non-cannabinoid solid ingredients; wherein said total non-cannabinoid solid ingredients are less than about 95%, about 90%, about 85%, about 80%, about 75%, about 70%, about 65%, about 60%, about 55%, about 50%, about 45%, about 40%, about 35%, about 30%, about 25%, or about 20% of the Cannabinoid Product by weight.

Embodiment 52. The cannabinoid daily dose of any one of embodiments 32-51, wherein the non-crystalline cannabinoid oil is loaded onto the colloidal silicon dioxide particles in the absence of any other solid ingredients.

Embodiment 53. The cannabinoid daily dose of any one of embodiments 32-52, wherein the Cannabinoid Product comprises less than 10%, 9%, 8%, 7%, 6%, 5%, 4%, 3%, 2%, 1% w/w cyclodextrin.

Embodiment 54. The cannabinoid daily dose of any one of embodiments 32-53, wherein the Cannabinoid Product does not comprise cyclodextrin.

Embodiment 55. The cannabinoid daily dose of any one of embodiments 32-54, wherein the Cannabinoid Product comprises less than 10%, 9%, 8%, 7%, 6%, 5%, 4%, 3%, 2%, 1% w/w emulsifier.

Embodiment 56. The cannabinoid daily dose of any one of embodiments 32-55, wherein the Cannabinoid Product does not comprise an emulsifier.

Embodiment 56.1 The cannabinoid daily dose of any one of embodiments 32-56, wherein the cannabinoid daily dose exhibits a linear dissolution rate as measured between 5-60 minutes in 1% Tween 80 media, Fasted State Simulated Intestinal Fluid (FaSSIF) media, and/or Fed State Simulated Intestinal Fluid (FeSSIF) media.

Embodiment 56.2 The cannabinoid daily dose of any one of embodiments 32-56.1, wherein at least 70% of the non-crystalline cannabinoid oil is loaded into the colloidal silicon dioxide storage particles.

Embodiment 57. A solid Cannabinoid Product comprising:
(a) non-crystalline cannabinoid oil; and
(b) colloidal silicon dioxide particles,
   wherein the non-crystalline cannabinoid oil is loaded into the colloidal silicon dioxide storage particles, and wherein the product exhibits a linear dissolution rate as measured in 1% Tween 80 media, Fasted State Simulated Intestinal Fluid (FaSSIF) media, and/or Fed State Simulated Intestinal Fluid (FeSSIF) media.

Embodiment 58. The solid Cannabinoid Product of embodiment 57, wherein the dissolution rate remains linear throughout 5-60 minutes after contacting the product with the 1% tween 80 media, FaSSIF media, and/or FeSSIF media.

Embodiment 59. The solid Cannabinoid Product of any one of embodiments 57-58, wherein the dissolution rate remains linear throughout a first phase, a second phase, and third phase after contacting the product with the 1% tween 80 media, FaSSIF media, and/or FeSSIF media.

Embodiment 60. The solid Cannabinoid Product of any one of embodiments 57-59, wherein the first phase comprises a period of 5-14 minutes after contacting the product with the 1% tween 80 media, FaSSIF media, and/or FeSSIF media, wherein the second phase comprises a period of 15-34 minutes after contacting the product with the 1% tween 80 media, FaSSIF media, and/or FeSSIF media, and wherein the third phase comprises a period of 35-60 minutes after contacting the product with the 1% tween 80 media, FaSSIF media, and/or FeSSIF media.

Embodiment 61. The solid Cannabinoid Product of any one of embodiments 57-60, wherein the linear dissolution rate comprises an R² value not less than about 0.90.

Embodiment 62. The solid Cannabinoid Product of any one of embodiments 57-61, wherein the Cannabinoid Product exhibits a linear absorption rate for at least 20 minutes after administration.

Embodiment 63. The solid Cannabinoid Product of any one of embodiments 57-62, wherein the Cannabinoid Product exhibits a linear absorption rate for at least 40 minutes after administration.

Embodiment 64. The solid Cannabinoid Product of any one of embodiments 57-63, wherein the Cannabinoid Product exhibits a linear absorption rate for at least 1 hour after administration.

Embodiment 65. The solid Cannabinoid Product of any one of embodiments 57-64, wherein the non-crystalline cannabinoid oil in the Cannabinoid Product is in solid form.

Embodiment 66. The solid Cannabinoid Product of any one of embodiments 57-65, wherein the non-crystalline cannabinoid oil is not diluted in a non-cannabis solvent.

Embodiment 67. The solid Cannabinoid Product of any one of embodiments 57-66, wherein the non-cannabis solvent is an alcohol.

Embodiment 68. The solid Cannabinoid Product of any one of embodiments 57-67, wherein the non-cannabis solvent is medium-chain triglycerides.

Embodiment 69. The solid Cannabinoid Product of any one of embodiments 57-68, wherein the non-cannabis solvent is castor oil.

Embodiment 70. The solid Cannabinoid Product of any one of embodiments 57-69, wherein the non-cannabis solvent is Kolliphor EL.

Embodiment 71. The solid Cannabinoid Product of any one of embodiments 57-70, comprising total non-cannabinoid solid ingredients; wherein said total non-cannabinoid solid ingredients are less than about 95%, about 90%, about 85%, about 80%, about 75%, about 70%, about 65%, about 60%, about 55%, about 50%, about 45%, about 40%, about 35%, about 30%, about 25%, or about 20% of the Cannabinoid Product by weight.

Embodiment 72. The solid Cannabinoid Product of any one of embodiments 57-71, wherein the non-crystalline cannabinoid oil is loaded onto the colloidal silicon dioxide particles in the absence of any other solid ingredients.

Embodiment 73. The solid Cannabinoid Product of any one of embodiments 57-72, wherein the Cannabinoid Product comprises less than 10%, 9%, 8%, 7%, 6%, 5%, 4%, 3%, 2%, 1% w/w cyclodextrin.

Embodiment 74. The solid Cannabinoid Product of any one of embodiments 57-73, wherein the Cannabinoid Product does not comprise cyclodextrin.

Embodiment 75. The solid Cannabinoid Product of any one of embodiments 57-74, wherein the Cannabinoid Product comprises less than 10%, 9%, 8%, 7%, 6%, 5%, 4%, 3%, 2%, 1% w/w emulsifier.

Embodiment 76. The solid Cannabinoid Product of any one of embodiments 57-75, wherein the Cannabinoid Product does not comprise an emulsifier.

Embodiment 77. The solid Cannabinoid Product of any one of embodiments 57-76, wherein the solid Cannabinoid Product has a hardness of from 50 Newtons (N) to 100 N.

Embodiment 78. A method of loading into a non-crystalline cannabinoid oil into a plurality of colloidal silicon dioxide particles comprising internal storage cavities, said method comprising the steps of:
(a) providing a cannabinoid;
(b) heating the cannabinoid, thereby producing a non-crystalline cannabinoid oil;
(c) providing a plurality of colloidal silicon dioxide particles comprising internal storage cavities; and
(d) contacting the non-crystalline cannabinoid oil with the plurality colloidal silicon dioxide particles comprising internal storage cavities,
thereby loading substantially all of the non-crystalline cannabinoid oil into the colloidal silicon dioxide particles comprising internal storage cavities.

Embodiment 79. The method of embodiment 78, wherein the non-crystalline cannabinoid oil loaded into the colloidal silicon dioxide particles is in solid form.

Embodiment 80. The method of any one of embodiments 78-79, wherein the non-crystalline cannabinoid oil is not diluted in a non-cannabis solvent.

Embodiment 81. The method of any one of embodiments 78-80, wherein the non-cannabis solvent is an alcohol.

Embodiment 82. The method of any one of embodiments 78-80, wherein the non-cannabis solvent is medium-chain triglycerides.

Embodiment 83. The method of any one of embodiments 78-80, wherein the non-cannabis solvent is castor oil.

Embodiment 84. The method of any one of embodiments 78-80, wherein the non-cannabis solvent is Kolliphor EL.

Embodiment 85. A method of making a solid Cannabinoid Product, said method comprising the steps of:
(a) providing a cannabinoid;
(b) heating the cannabinoid, thereby producing non-crystalline cannabinoid oil;
(c) providing a plurality of colloidal silicon dioxide particles comprising internal storage cavities;
(d) mixing the non-crystalline cannabinoid oil and colloidal silicon dioxide particles comprising internal storage cavities;
(e) adding a filler and disintegrant to the mixture of non-crystalline cannabinoid oil and colloidal silicon dioxide particles comprising internal storage cavities; and
(f) adding a lubricant.

Embodiment 86. The method of embodiment 85, wherein the non-crystalline cannabinoid oil loaded into the colloidal silicon dioxide particles is in solid form.

Embodiment 87. The method of any one of embodiments 85-86, wherein the non-crystalline cannabinoid oil is not diluted in a non-cannabis solvent.

Embodiment 88. The method of any one of embodiments 86-87, wherein the non-cannabis solvent is an alcohol.

Embodiment 89. The method of any one of embodiments 86-87, wherein the non-cannabis solvent is medium-chain triglycerides.

Embodiment 90. The method of any one of embodiments 86-87, wherein the non-cannabis solvent is castor oil.

Embodiment 91. The method of any one of embodiments 86-87, wherein the non-cannabis solvent is Kolliphor EL.

Embodiment 92. The method of any one of embodiments 86-91, wherein the solid Cannabinoid Product further comprises total non-cannabinoid solid ingredients; wherein said total non-cannabinoid solid ingredients are less than about 95%, about 90%, about 85%, about 80%, about 75%, about 70%, about 65%, about 60%, about 55%, about 50%, about 45%, about 40%, about 35%, about 30%, about 25%, or about 20% of the Cannabinoid Product by weight.

Embodiment 93. The method of any one of embodiments 86-92, wherein the non-crystalline cannabinoid oil is loaded onto the colloidal silicon dioxide particles in the absence of any other solid ingredients.

Embodiment 94. The method of any one of embodiments 86-93, wherein the solid Cannabinoid Product comprises less than 10%, 9%, 8%, 7%, 6%, 5%, 4%, 3%, 2%, 1% w/w cyclodextrin.

Embodiment 95. The method of any one of embodiments 86-94, wherein the solid Cannabinoid Product does not comprise cyclodextrin.

Embodiment 96. The method of any one of embodiments 86-95, wherein the solid Cannabinoid Product comprises less than 10%, 9%, 8%, 7%, 6%, 5%, 4%, 3%, 2%, 1% w/w emulsifier.

Embodiment 97. The method of any one of embodiments 86-96, wherein the solid Cannabinoid Product does not comprise an emulsifier.

Embodiment 98. The method of any one of embodiments 86-97, wherein the solid Cannabinoid Product has a hardness of from 50 Newtons (N) to 100 N.

Embodiment 99. A method of making a Cannabinoid Product, said method comprising the steps of:
(a) providing a cannabinoid;
(b) heating the cannabinoid, thereby producing non-crystalline cannabinoid oil;
(c) providing a plurality of colloidal silicon dioxide particles comprising internal storage cavities;
(d) mixing the non-crystalline cannabinoid oil and colloidal silicon dioxide particles comprising internal storage cavities; and
(e) adding a lubricant.

Embodiment 100. The method of embodiment 99, wherein the non-crystalline cannabinoid oil loaded into the colloidal silicon dioxide particles is in solid form.

Embodiment 101. The method of any one of embodiments 99-100, wherein the non-crystalline cannabinoid oil is not diluted in a non-cannabis solvent.

Embodiment 102. The method of any one of embodiments 99-101, wherein the non-cannabis solvent is an alcohol.

Embodiment 103. The method of any one of embodiments 99-102, wherein the non-cannabis solvent is medium-chain triglycerides.

Embodiment 104. The method of any one of embodiments 99-103, wherein the non-cannabis solvent is castor oil.

Embodiment 105. The method of any one of embodiments 99-104, wherein the non-cannabis solvent is Kolliphor EL.

Embodiment 106. The method of any one of embodiments 99-105, wherein the solid Cannabinoid Product further comprises total non-cannabinoid solid ingredients; wherein said total non-cannabinoid solid ingredients are less than about 95%, about 90%, about 85%, about 80%, about 75%, about 70%, about 65%, about 60%, about 55%, about 50%, about 45%, about 40%, about 35%, about 30%, about 25%, or about 20% of the Cannabinoid Product by weight.

Embodiment 107. The method of any one of embodiments 99-106, wherein the non-crystalline cannabinoid oil is loaded onto the colloidal silicon dioxide particles in the absence of any other solid ingredients.

Embodiment 108. The method of any one of embodiments 99-107, wherein the solid Cannabinoid Product comprises less than 10%, 9%, 8%, 7%, 6%, 5%, 4%, 3%, 2%, 1% w/w cyclodextrin.

Embodiment 109. The method of any one of embodiments 99-108, wherein the solid Cannabinoid Product does not comprise cyclodextrin.

Embodiment 110. The method of any one of embodiments 99-109, wherein the solid Cannabinoid Product comprises less than 10%, 9%, 8%, 7%, 6%, 5%, 4%, 3%, 2%, 1% w/w emulsifier.

Embodiment 111. The method of any one of embodiments 99-110, wherein the solid Cannabinoid Product does not comprise an emulsifier.

Embodiment 112. The method of any one of embodiments 98-111, wherein the solid Cannabinoid Product has a hardness of from 50 Newtons (N) to 100 N.

Embodiment 113. A Cannabinoid Product comprising:
(a) a non-crystalline cannabinoid oil; and
(b) colloidal silicon dioxide particles.

Embodiment 114. The Cannabinoid Product of embodiment 113, wherein the non-crystalline cannabinoid oil and colloidal silicon dioxide particles are provided as a mixture.

Embodiment 115. A Cannabinoid Product comprising:
(a) a non-crystalline cannabinoid oil;
(b) colloidal silicon dioxide particles;
(c) a filler comprising silicified microcrystalline cellulose or mannitol;
(d) a disintegrant comprising sodium starch glycolate or croscarmellose sodium; and
(e) a lubricant comprising sodium stearyl fumarate.

Embodiment 116. The Cannabinoid Product of embodiment 115, wherein the non-crystalline cannabinoid oil and colloidal silicon dioxide particles are provided as a mixture.

Embodiment 117. The Cannabinoid Product of any of embodiments 115-116, comprising from about 10% to about 60% non-crystalline cannabinoid oil by weight.

Embodiment 118. The Cannabinoid Product of any of embodiments 115-117, comprising from about 19.4% to about 41.9% non-crystalline cannabinoid oil by weight.

Embodiment 119. The Cannabinoid Product of any of embodiments 115-118, wherein the filler is silicified microcrystalline cellulose.

Embodiment 120. The Cannabinoid Product of any of embodiments 115-119, comprising from about 30% to about 60% silicified microcrystalline cellulose by weight.

Embodiment 121. The Cannabinoid Product of any of embodiments 115-120, comprising about 46.6% silicified microcrystalline cellulose by weight.

Embodiment 122. The Cannabinoid Product of any of embodiments 115-121, wherein the filler is mannitol.

Embodiment 123. The Cannabinoid Product of any of embodiments 115-122, comprising from about 5% to about 60% mannitol by weight.

Embodiment 124. The Cannabinoid Product of any of embodiments 115-123, comprising from about 12.3% to about 43.4% mannitol by weight.

Embodiment 125. The Cannabinoid Product of any of embodiments 115-124, comprising from about 20% to about 70% colloidal silicon dioxide particles by weight.

Embodiment 126. The Cannabinoid Product of any of embodiments 115-125, comprising from about 29.1% to about 58.2% colloidal silicon dioxide particles by weight.

Embodiment 127. The Cannabinoid Product of any of embodiments 115-126, wherein the disintegrant is sodium starch glycolate.

Embodiment 128. The Cannabinoid Product of any of embodiments 115-127, comprising from about 0.5% to about 10% sodium starch glycolate by weight.

Embodiment 129. The Cannabinoid Product of any of embodiments 115-128, comprising from about 2.0% to about 5.5% sodium starch glycolate by weight.

Embodiment 130. The Cannabinoid Product of any of embodiments 115-129, wherein the disintegrant is croscarmellose sodium.

Embodiment 131. The Cannabinoid Product of any of embodiments 115-130, comprising from about 0.5% to about 5% croscarmellose sodium by weight.

Embodiment 132. The Cannabinoid Product of any of embodiments 115-131, comprising about 1.9% croscarmellose sodium by weight.

Embodiment 133. The Cannabinoid Product of any of embodiments 115-132, comprising from about 0.5% to about 10% sodium stearyl fumarate by weight.

Embodiment 134. The Cannabinoid Product of any of embodiments 115-133, comprising from about 1% to about 2.7% sodium stearyl fumarate by weight.

Embodiment 135. The Cannabinoid Product of any of embodiments 115-134, comprising:
(a) about 41.9% non-crystalline cannabinoid oil by weight;
(b) about 41.9% colloidal silicon dioxide particles by weight;
(c) about 12.3% mannitol by weight;
(d) about 2% sodium starch glycolate by weight; and
(e) about 2% sodium stearyl fumarate by weight.

Embodiment 136. The Cannabinoid Product of any of embodiments 115-134, comprising:
(a) about 22.1% non-crystalline cannabinoid oil by weight;
(b) about 58.2% colloidal silicon dioxide particles by weight;
(c) about 14.3% mannitol by weight;
(d) about 2.7% sodium starch glycolate by weight; and
(e) about 2.7% sodium stearyl fumarate by weight.

Embodiment 137. The Cannabinoid Product of any of embodiments 115-134, comprising:
(a) about 22.1% non-crystalline cannabinoid oil by weight;
(b) about 29.1% colloidal silicon dioxide particles by weight;
(c) about 40.7% mannitol by weight;
(d) about 5.5% sodium starch glycolate by weight; and
(e) about 2.7% sodium stearyl fumarate by weight.

Embodiment 138. The Cannabinoid Product of any of embodiments 115-134, comprising:
(a) about 22.1% non-crystalline cannabinoid oil by weight;
(b) about 29.1% colloidal silicon dioxide particles by weight;
(c) about 43.4% mannitol by weight;
(d) about 2.7% sodium starch glycolate by weight; and
(e) about 2.7% sodium stearyl fumarate by weight.

Embodiment 139. The Cannabinoid Product of any of embodiments 115-134, comprising:
(a) about 19.4% non-crystalline cannabinoid oil by weight;
(b) about 31.1% colloidal silicon dioxide particles by weight;
(c) about 46.6% silicified microcrystalline cellulose by weight;
(d) about 1.9% croscarmellose sodium by weight; and
(e) about 1% sodium stearyl fumarate by weight.

Embodiment 140. The Cannabinoid Product of any one of embodiments 115-139, further comprising a cannabinoid isolate.

Embodiment 141. The Cannabinoid Product of any one of embodiments 115-140, wherein the cannabinoid isolate is cannabidiol (CBD) isolate or cannabigerol (CBG) isolate.

Embodiment 142. The Cannabinoid Product of any one of embodiments 115-141, further comprising a flavoring.

Embodiment 143. The Cannabinoid Product of embodiments 115-142, wherein the flavoring is a terpene.

Embodiment 144. The Cannabinoid Product of embodiments 115-143, wherein the terpene is myrcene, linalool, or a combination thereof.

Embodiment 145. The Cannabinoid Product of any one of embodiments 115-144, wherein the non-crystalline cannabinoid oil is cannabinoid distillate.

Embodiment 146. The Cannabinoid Product of any one of embodiments 115-145, wherein the non-crystalline cannabinoid oil and the colloidal silicon dioxide particles are mixed in a high-shear mixer before the addition of the filler, disintegrant, or lubricant.

Embodiment 147. The Cannabinoid Product of any one of embodiments 115-146, wherein the lubricant is added after the filler and disintegrant.

Embodiment 148. The Cannabinoid Product of any one of embodiments 115-147, wherein the colloidal silicon dioxide particles have an average particle size of 20-60 μm.

Embodiment 149. The Cannabinoid Product of any one of embodiments 115-148, wherein the colloidal silicon dioxide particles contain pores with a volume of 1.5-1.9 milliliter (mL)/gram (g).

Embodiment 150. A Cannabinoid Product comprising:
(a) a non-crystalline cannabinoid oil;
(b) colloidal silicon dioxide particles; and
(c) a lubricant comprising sodium stearyl fumarate.

Embodiment 151. The Cannabinoid Product of embodiment 150, wherein the non-crystalline cannabinoid oil and colloidal silicon dioxide particles are provided as a mixture.

Embodiment 152. The Cannabinoid Product of any one of embodiments 150-151, comprising from about 0.5% to about 20% non-crystalline cannabinoid oil by weight.

Embodiment 153. The Cannabinoid Product of any one of embodiments 150-152, comprising from about 1% to about 10% non-crystalline cannabinoid oil by weight.

Embodiment 154. The Cannabinoid Product of any one of embodiments 150-153, comprising about 2% non-crystalline cannabinoid oil by weight.

Embodiment 155. The Cannabinoid Product of any one of embodiments 150-154, comprising about 8% non-crystalline cannabinoid oil by weight.

Embodiment 156. The Cannabinoid Product of any one of embodiments 150-155, comprising from about 25% to about 50%, from about 25% to about 35%, or from about 35% to about 45% colloidal silicon dioxide particles by weight.

Embodiment 157. The Cannabinoid Product of any one of embodiments 150-156, comprising about 32% colloidal silicon dioxide particles by weight.

Embodiment 158. The Cannabinoid Product of any one of embodiments 150-157, comprising about 39% colloidal silicon dioxide particles by weight.

Embodiment 159. The Cannabinoid Product of any one of embodiments 150-158, comprising from about 0.1% to about 1% sodium stearyl fumarate by weight.

Embodiment 160. The Cannabinoid Product of any one of embodiments 150-159, comprising about 0.4% sodium stearyl fumarate by weight.

Embodiment 161. The Cannabinoid Product of any one of embodiments 150-160, further comprising a cannabinoid isolate.

Embodiment 162. The Cannabinoid Product of any one of embodiments 150-161, wherein the cannabinoid isolate is cannabidiol (CBD) isolate or cannabigerol (CBG) isolate.

Embodiment 163. The Cannabinoid Product of any one of embodiments 150-162, comprising from 20% to about 60%, from about 20% to about 40%, or from about 40% to about 60% cannabinoid isolate by weight.

Embodiment 164. The Cannabinoid Product of any one of embodiments 150-163, further comprising a filler.

Embodiment 165. The Cannabinoid Product of any one of embodiments 150-164, wherein the filler is silicified microcrystalline cellulose.

Embodiment 166. The Cannabinoid Product of any one of embodiments 150-165, comprising from about 20% to about 45% filler by weight.

Embodiment 167. The Cannabinoid Product of any one of embodiments 150-166, comprising about 25% filler by weight.

Embodiment 168. The Cannabinoid Product of any one of embodiments 150-167, further comprising a flavoring.

Embodiment 169. The Cannabinoid Product of any one of embodiments 150-168, wherein the flavoring is a terpene.

Embodiment 170. The Cannabinoid Product of any one of embodiments 150-169, wherein the terpene is myrcene, linalool, or a combination thereof.

Embodiment 171. The Cannabinoid Product of any one of embodiments 150-170, comprising:
(a) about 8.1% non-crystalline cannabinoid oil by weight;
(b) about 39% colloidal silicon dioxide particles by weight; and
(c) about 0.4% sodium stearyl fumarate by weight.

Embodiment 172. The Cannabinoid Product of any one of embodiments 150-170, comprising:
(a) about 2.2% non-crystalline cannabinoid oil by weight of the Cannabinoid Product;
(b) about 32.4% colloidal silicon dioxide particles by weight; and
(c) about 0.4% sodium stearyl fumarate by weight.

Embodiment 173. The Cannabinoid Product of any one of embodiments 150-170, comprising:
(a) about 8.1% non-crystalline cannabinoid oil by weight;
(b) about 39% colloidal silicon dioxide particles by weight;
(c) about 0.4% sodium stearyl fumarate by weight;
(d) about 40.4% cannabidiol (CBD) isolate by weight;
(e) about 11.7% cannabigerol (CBG) isolate by weight;
(f) about 0.2% myrcene by weight; and
(g) about 0.2% linalool by weight.

Embodiment 174. The Cannabinoid Product of any one of embodiments 150-170, comprising:
(a) about 8.1% non-crystalline cannabinoid oil by weight;
(b) about 39% colloidal silicon dioxide particles by weight;
(c) about 0.4% sodium stearyl fumarate by weight;
(d) about 40.4% cannabidiol (CBD) isolate by weight; and
(e) about 11.7% cannabigerol (CBG) isolate by weight;

Embodiment 175. The Cannabinoid Product of any one of embodiments 150-170, comprising:
(a) about 2.2% non-crystalline cannabinoid oil by weight;
(b) about 32.4% colloidal silicon dioxide particles by weight;
(c) about 0.4% sodium stearyl fumarate by weight; and
(d) about 34.8% silicified microcrystalline cellulose by weight.

Embodiment 176. The Cannabinoid Product of any one of embodiments 150-175, wherein the non-crystalline cannabinoid oil is a cannabinoid distillate.

Embodiment 177. The Cannabinoid Product of any one of embodiments 150-176, wherein the non-crystalline cannabinoid oil and the colloidal silicon dioxide particles are mixed in a high-shear mixer before the addition of the lubricant.

Embodiment 178. The Cannabinoid Product of any one of embodiments 150-177, wherein the colloidal silicon dioxide particles have an average particle size of from about 20 μm to about 60 μm.

Embodiment 179. The Cannabinoid Product of any one of embodiments 150-178, wherein the colloidal silicon dioxide particles contain pores with a volume of from about 1.5 milliliter (mL)/gram (g) to about 1.9 mL/g.

Embodiment 180. A tablet comprising the Cannabinoid Product of any one of embodiments 150-179.

Embodiment 181. The tablet of embodiment 180, wherein the tablet has a hardness of from 50 Newtons (N) to 100 N.

Embodiment 182. The tablet of any one of embodiments 180-181, wherein the tablet has a hardness of about 65 N.

Embodiment 183. The tablet of any one of embodiments 180-182, wherein the tablet has a compression force of from 1 kiloNewtons (kN) to 30 kN.

Embodiment 184. The tablet of any one of embodiments 180-183, wherein the tablet has a compression force of from about 10 kN to 16 kN.

Embodiment 185. The tablet of any one of embodiments 180-184, wherein at least 18% of the tablet dissolves in fed state simulated intestinal fluid or fasted state simulated intestinal fluid within one hour.

Embodiment 186. The tablet of any one of embodiments 180-185, wherein from 60% to about 80% of the tablet dissolves.

Embodiment 187. The tablet of any one of embodiments 180-186, wherein from 70% to 80% of the tablet dissolves.

Embodiment 188. The tablet of any one of embodiments 180-187, wherein at least 36.5% of the tablet dissolves in polysorbate 80.

Embodiment 189. The tablet of any one of embodiments 180-188, wherein the tablet disintegrates in from 1 minute to about 10 minutes in 2% w/v sodium lauryl sulfate.

Embodiment 190. The tablet of any one of embodiments 180-189, wherein the tablet dissolves at least two times, at least three times, at least four times, at least five times, or at least ten times faster than a tablet containing a Cannabinoid Product lacking colloidal silicon dioxide particles.

Embodiment 191. The tablet of any one of embodiments 180-190, wherein the tablet has a weight of from about 100 to about 800 mg.

Embodiment 192. The tablet of any one of embodiments 180-191, wherein the tablet comprises from about 0.1% to about 30% of non-crystalline cannabinoid oil by weight.

Embodiment 193. The tablet of any one of embodiments 180-192, wherein the tablet is an orodispersable tablet.

Embodiment 194. A capsule comprising the Cannabinoid Product of any one of embodiments 150-179.

Embodiment 195. The capsule of embodiment 194, wherein the capsule comprises a shell comprising gelatin or hydroxypropylmethylcellulose.

Embodiment 196. The capsule of any one of embodiments 194-195, wherein the capsule disintegrates within about 2 minutes in an aqueous solvent.

Embodiment 197. The capsule of any one of embodiments 194-196, wherein at least 18% of the capsule dissolves in fed state simulated intestinal fluid or fasted state simulated intestinal fluid within one hour.

Embodiment 198. The capsule of any one of embodiments 194-197, wherein from 18% to about 60% of the capsule dissolves.

Embodiment 199. The capsule of any one of embodiments 194-198, wherein from 18.1% to 42.3% of the capsule dissolves.

Embodiment 200. The capsule of any one of embodiments 194-199, wherein at least 36.5% of the capsule dissolves within one hour.

Embodiment 201. The capsule of any one of embodiments 194-200, wherein the capsule dissolves at least two times, at least three times, at least four times, at least five times, or at least ten times faster than a capsule containing a Cannabinoid Product lacking colloidal silicon dioxide particles.

Embodiment 202. The capsule of any one of embodiments 194-201, wherein the capsule comprises from about 1% to about 50% non-crystalline cannabinoid oil by weight.

Embodiment 203. A method of making the Cannabinoid Product of any one of embodiments 150-179, comprising:
(a) mixing the non-crystalline cannabinoid oil and colloidal silicon dioxide particles;
(b) adding the filler and disintegrant to the mixture of non-crystalline cannabinoid oil and colloidal silicon dioxide particles; and
(c) adding the lubricant.

Embodiment 204. The method of embodiment 203, wherein step (a) is performed before step (b), and step (b) is performed before step (c).

Embodiment 205. The method of any one of embodiments 203-204, wherein the non-crystalline cannabinoid oil is heated before the extract oil is mixed with the colloidal silicon dioxide particles.

Embodiment 206. The method of any one of embodiments 203-205, wherein the non-crystalline cannabinoid oil and colloidal silicon dioxide particles are heated after mixing.

Embodiment 207. The method of any one of embodiments 203-206, wherein the non-crystalline cannabinoid oil is diluted in a solvent before mixing with colloidal silicon dioxide particles.

Embodiment 208. The method of any one of embodiments 203-207, wherein the solvent is ethanol.

Embodiment 209. The method of any one of embodiments 203-208, wherein the non-crystalline cannabinoid oil is heated to at least 30° C., at least 40° C., at least 50° C., at least 60° C., at least 70° C., at least 80° C., or at least 90° C.

Embodiment 210. The method of any one of embodiments 203-209, wherein the non-crystalline cannabinoid oil is added to the colloidal silicon dioxide particles.

Embodiment 211. The method of any one of embodiments 203-210, wherein the colloidal silicon dioxide particles are added to the cannabinoid non-crystalline cannabinoid oil.

Embodiment 212. The method of any one of embodiments 203-211, wherein the non-crystalline cannabinoid oil and colloidal silicon dioxide particles are mixed in a high-shear mixer.

Embodiment 213. The method of any one of embodiments 203-212, wherein the non-crystalline cannabinoid oil and colloidal silicon dioxide particles are mixed for at least 30 seconds, at least 1 minute, at least 5 minutes, at least 10 minutes, at least 15 minutes, or at least 20 minutes.

Embodiment 214. The method of any one of embodiments 203-213, wherein the non-crystalline cannabinoid oil and colloidal silicon dioxide particles are mixed at from 1000 to 2000 revolutions per minute (rpm).

Embodiment 215. The method of any one of embodiments 203-214, wherein the high-shear mixer is a grinder.

Embodiment 216. A method of making a tablet comprising compressing a Cannabinoid Product of any one of embodiments 150-179 in a tablet press.

Embodiment 217. The method of embodiment 216, wherein the tablet is about 100 to 800 mg.

Embodiment 218. The method of any one of embodiments 216-217, wherein the tablet comprises from about 0.1% to about 30% of non-crystalline cannabinoid oil by weight.

Embodiment 219. The method of any one of embodiments 216-218, wherein the tablet is an orodispersable tablet.

Embodiment 220. A method of making the Cannabinoid Product of any one of embodiments 150-219, comprising:
(a) mixing the non-crystalline cannabinoid oil and colloidal silicon dioxide particles; and
(b) adding the lubricant.

Embodiment 221. The method of embodiment 220, further comprising:
(c) adding a filler.

Embodiment 222. The method of any one of embodiments 220-221, wherein the filler is silicified microcrystalline cellulose.

Embodiment 223. The method of any one of embodiments 220-222, wherein the non-crystalline cannabinoid oil is diluted in a solvent before mixing with colloidal silicon dioxide particles.

Embodiment 224. The method of any one of embodiments 220-223, wherein the solvent is ethanol.

Embodiment 225. The method of any one of embodiments 220-224, wherein step (a) is performed before step (b).

Embodiment 226. The method of any one of embodiments 220-225, wherein step (a) is performed before step (b), and step (b) is performed before step (c).

Embodiment 227. The method of any one of embodiments 220-226, wherein step (a) is performed before step (c), and step (c) is performed before step (b).

Embodiment 228. The method of any one of embodiments 220-227, wherein the non-crystalline cannabinoid oil is heated before the oil is mixed with the colloidal silicon dioxide particles.

Embodiment 229. The method of any one of embodiments 220-228, wherein the non-crystalline cannabinoid oil and colloidal silicon dioxide particles are heated after mixing.

Embodiment 230. The method of any one of embodiments 220-229, wherein the non-crystalline cannabinoid oil is heated to at least 30° C., at least 40° C., at least 50° C., at least 60° C., at least 70° C., at least 80° C., or at least 90° C.

Embodiment 231. The method of any one of embodiments 220-230, wherein the non-crystalline cannabinoid oil is added to the colloidal silicon dioxide particles.

Embodiment 232. The method of any one of embodiments 220-231, wherein the colloidal silicon dioxide particles are added to the cannabinoid non-crystalline cannabinoid oil.

Embodiment 233. The method of any one of embodiments 220-232, wherein the non-crystalline cannabinoid oil and colloidal silicon dioxide particles are mixed in a high-shear mixer.

Embodiment 234. The method of any one of embodiments 220-233, wherein the non-crystalline cannabinoid oil and colloidal silicon dioxide particles are mixed for at least 30 seconds, at least 1 minute, at least 5 minutes, at least 10 minutes, at least 15 minutes, or at least 20 minutes.

Embodiment 235. The method of any one of embodiments 220-234, wherein the non-crystalline cannabinoid oil and colloidal silicon dioxide particles are mixed at a speed of from about 1000 revolutions per minute (rpm) to about 2000 rpm.

Embodiment 236. The method of any one of embodiments 220-235, wherein the lubricant is mixed with a mixture containing non-crystalline cannabinoid oil, colloidal silicon dioxide particles, filler, and disintegrant for from 1 minute (min) to about 2 min.

Embodiment 237. The method of any one of embodiments 220-236, wherein the high-shear mixer is a grinder.

Embodiment 238. A method of making a capsule, comprising filling a capsule shell with the Cannabinoid Product of any one of embodiments 150-237.

Embodiment 239. The method of embodiment 238, wherein the capsule shell comprises gelatin or hydroxypropylmethylcellulose.

Embodiment 240. The method of any one of embodiments 238-239, wherein the capsule comprises from about 1% to about 50% non-crystalline cannabinoid oil by weight.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world. The following documents are also incorporated by reference herein in their entirety: International Publication No. 2003/082246; U.S. Publication No. 2019/0060381; U.S. Publication No. 2017/0368020; International Publication No. 2019/216832; U.S. Pat. No. 11,077,086; International Publication No. 2021/151169; Sperry et al. Precis. Nanomed. 2021 December; 4 (4): 851-878; Pandey et al. Pharm Dev Technol. 2013 February; 18 (1): 296-304; and Sopper et al. Sci Pharm. 2021, 89, 35.

What is claimed is:

1. A solid Cannabinoid Product comprising:
(a) non-crystalline cannabinoid oil; and
(b) colloidal silicon dioxide particles comprising internal storage cavities,
wherein the non-crystalline cannabinoid oil is loaded into the colloidal silicon dioxide storage particles, and wherein the loaded non-crystalline cannabinoid oil to colloidal silicon dioxide particle ratio is about 0.5:1 to 3:1 by weight.

2. The solid Cannabinoid Product of claim 1, wherein the non-crystalline cannabinoid oil comprises at least 80% cannabinoid by weight.

3. The solid Cannabinoid Product of claim 1, wherein the non-crystalline cannabinoid oil is not diluted in a non-cannabis solvent.

4. The solid Cannabinoid Product of claim 1, comprising total non-cannabinoid solid ingredients; wherein said total non-cannabinoid solid ingredients are less than about 80% of the Cannabinoid Product by weight.

5. The solid Cannabinoid Product of claim 1, wherein the non-crystalline cannabinoid oil is loaded onto the colloidal silicon dioxide particles in the absence of any other solid ingredients.

6. The solid Cannabinoid Product of claim 1, wherein the Cannabinoid Product comprises less than 10% w/w cyclodextrin.

7. The solid Cannabinoid Product of claim 1, wherein the Cannabinoid Product comprises less than 10% w/w emulsifier.

8. The solid Cannabinoid Product of claim 1, wherein the solid Cannabinoid Product has a hardness of from 50 Newtons (N) to 100 N.

9. The solid Cannabinoid Product of claim 1, wherein the Cannabinoid Product exhibits a linear dissolution rate as measured between 5-60 minutes in 1% Tween 80 media, Fasted State Simulated Intestinal Fluid (FaSSIF) media, and/or Fed State Simulated Intestinal Fluid (FeSSIF) media.

10. A solid Cannabinoid Product comprising:
(a) non-crystalline cannabinoid oil; and
(b) colloidal silicon dioxide particles comprising internal storage cavities,
wherein the non-crystalline cannabinoid oil is substantially loaded into the colloidal silicon dioxide storage particles, and wherein the non-crystalline cannabinoid oil content is at least 30% w/w of the colloidal silicon dioxide particle content.

11. The solid Cannabinoid Product of claim 10, wherein the non-crystalline cannabinoid oil is not diluted in a non-cannabis solvent.

12. The solid Cannabinoid Product of claim 10, wherein the Cannabinoid Product comprises less than 10% w/w cyclodextrin.

13. The solid Cannabinoid Product of claim 10, wherein the Cannabinoid Product comprises less than 10% w/w emulsifier.

14. The solid Cannabinoid Product of claim 10, wherein the solid Cannabinoid Product has a hardness of from 50 Newtons (N) to 100 N.

15. The solid Cannabinoid Product of claim 10, wherein at least 70% of the non-crystalline cannabinoid oil is loaded into the colloidal silicon dioxide storage particles.

16. The solid Cannabinoid Product of claim 10, wherein the Cannabinoid Product exhibits a linear dissolution rate as measured between 5-60 minutes in 1% Tween 80 media, Fasted State Simulated Intestinal Fluid (FaSSIF) media, and/or Fed State Simulated Intestinal Fluid (FeSSIF) media.

17. A method of making a solid Cannabinoid Product, said method comprising the steps of:
(a) providing a cannabinoid;
(b) heating the cannabinoid, thereby producing a non-crystalline cannabinoid oil;
(c) providing a plurality of colloidal silicon dioxide particles comprising internal storage cavities;
(d) mixing the non-crystalline cannabinoid oil and colloidal silicon dioxide particles comprising internal storage cavities; and
(e) adding a lubricant.
